(19) United States
(12) Patent Application Publication
(10) Pub. No.: US 2026/0083129 A1
(43) Pub. Date: Mar. 26, 2026

(54) PYRAZOLE AMIDE INSECTICIDES

(71) Applicant: FMC CORPORATION, Philadelphia, PA (US)

(72) Inventors: Jeffrey Keith LONG, Newark, DE (US); Thomas Francis PAHUTSKI, Jr., Newark, DE (US); George Philip LAHM, Newark, DE (US); Jyoti NANDI, Newark, DE (US)

(21) Appl. No.: 19/112,822

(22) PCT Filed: Sep. 21, 2023
(86) PCT No.: PCT/US2023/033366

§ 371 Date: Mar. 18, 2025

Related U.S. Application Data
(60) Provisional application No. 63/409,393, filed on Sep. 23, 2022.

(51) Int. Cl. A01N 43/54; A01N 43/40; A01P 7/04; C07D 401/04; C07D 403/04

(52) U.S. Cl. CPC A01N 43/54; A01N 43/40; A01P 7/04; C07D 401/04; C07D 403/04

(57) ABSTRACT

Disclosed are compounds of Formula 1,

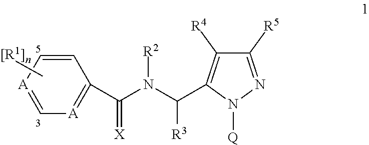

and A, R¹, R², R³, R⁴, R⁵, Q and X are as defined in the disclosure. Also disclosed are compositions containing the compounds of Formula 1 and methods for controlling and combating an invertebrate pest comprising contacting the invertebrate pest or its environment with a biologically effective amount of a compound or a composition of the invention.

### CROSS-REFERENCE TO RELATED APPLICATIONS

This application claims the benefit of U.S. Provisional Application No. 63/409,393 filed Sep. 23, 2022.

### FIELD

This invention relates to novel pyrazole compounds and compositions suitable for agronomic and nonagronomic uses, and methods of their use for controlling and combating invertebrate pests such as arthropods in both agronomic and nonagronomic environments.

### BACKGROUND

The control of invertebrate pests is extremely important in achieving high crop efficiency. Damage by invertebrate pests to growing and stored agronomic crops can cause significant reduction in productivity and thereby result in increased costs to the consumer. The control of invertebrate pests in forestry, greenhouse crops, ornamentals, nursery crops, stored food and fiber products, household, turf, wood products, and public health is also important. Many products are commercially available for these purposes, but the need continues for new compounds that are more effective, less costly, less toxic, environmentally safer or have different sites of action.

### SUMMARY

This disclosure is directed to compounds of Formula 1 (including all geometric and stereoisomers), N-oxides, and salts thereof, and compositions containing them and their use for controlling and combating invertebrate pests:

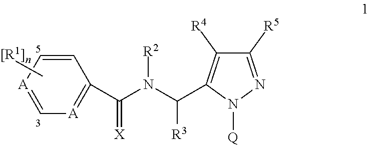

wherein
X is O or S;
each A is independently CH, N or CR¹;
each R¹ is independently H, amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or
each R¹ is independently phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or each R¹ is independently C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or R² is C(O)R⁷, C(O)OR⁷, NR⁷R⁸, OR⁷, S(O)ₚR⁹ or SO₂NR⁷R⁸; R³ is C₁-C₃ alkyl or C₁-C₃ haloalkyl;
R⁴ is hydrogen, cyano, halogen, nitro, C(S)NH₂, SCN, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R⁴ is C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸;
R⁵ is hydrogen, cyano, halogen, nitro, C(S)NH₂, SCN, C₁-C₆-alkyl, C₂-C₆ alkenyl, C₂-C₆ alkynyl C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R⁵ is C(O)R⁷, CR⁷(═NO)R⁸, C(O)OR⁷, C(O)NR⁷R⁸, NR⁷R⁸, NR⁸C(O)R⁷, NR⁸C(O)OR⁷, NR¹¹SO₂NR⁷R⁸, OR⁷, S(O)ₚR⁹, or SO₂NR⁷R⁸;
R⁶ is independently halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸, or S(═NR)OₚR⁷;
R⁷ is independently hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R¹²; or R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R¹²;
R⁸ is independently hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
when R⁷ and R⁸ are on N, R⁷ and R⁸ can form a ring of 3 to 7 carbons, optionally including oxygen, S(O)p or NR⁷;
R⁹ is independently C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶;
or R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
each R¹⁰ is independently H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
each R¹¹ is independently hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
R¹² is halogen, hydroxy, cyano, nitro, SF₅, C(O)NH₂, C(S)NH₂, C₁-C₃ alkyl, C₁-C₃ alkoxy, C₂-C₆ alkenyl, C₂-C₆ alkynyl, C₃-C₇ cycloalkyl, C₄-C₈ cycloalkylalkyl, wherein each alkyl, alkoxy, alkenyl, alkynyl, cycloalkyl or cycloalkylalkyl is optionally substituted with halogen or cyano;
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸;
n is 1, 2, or 3;
p is 0, 1, or 2.

This invention also provides a composition comprising a compound of Formula 1 and at least one additional component selected from the group consisting of surfactants, solid diluents and liquid diluents. In one embodiment, this invention also provides a composition for controlling and combating an invertebrate pest comprising a compound of Formula 1 and at least one additional component selected from the group consisting of surfactants, solid diluents and liquid diluents, said composition optionally further comprising at least one additional biologically active compound or agent.

This invention provides a method for controlling and combating an invertebrate pest comprising contacting the invertebrate pest or its environment with a biologically effective amount of a compound of Formula 1 (e.g., as a composition described herein). This invention also relates to such method wherein the invertebrate pest or its environment is contacted with a composition comprising a biologically effective amount of a compound of Formula 1, and at least one additional component selected from the group consisting of surfactants, solid diluents and liquid diluents, said composition optionally further comprising a biologically effective amount of at least one additional biologically active compound or agent.

This invention also provides a method for protecting a seed from an invertebrate pest comprising contacting the seed with a biologically effective amount of a compound of Formula 1 (e.g., as a composition described herein). This invention also relates to the treated seed.

This invention also provides a method for increasing vigor of a crop plant comprising contacting the crop plant, the seed from which the crop plant is grown or the locus (e.g., growth medium) of the crop plant with a biologically effective amount of a compound of Formula 1 (e.g., as a composition described herein).

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods, nematodes and helminths of economic importance as pests. The term “arthropod” includes insects, mites, spiders, scorpions, centipedes, millipedes, pill bugs and symphylans. The term “gastropod” includes snails, slugs and other Stylommatophora. The term “nematode” includes members of the phylum Nematoda.

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of maize or corn, soybeans and other legumes, rice, cereal (e.g., wheat, oats, barley, rye and rice), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (e.g., berries and cherries) and other specialty crops (e.g., canola, sunflower and olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, and public health applications.

The term “crop vigor” refers to rate of growth or biomass accumulation of a crop plant. An “increase in vigor” refers to an increase in growth or biomass accumulation in a crop plant relative to an untreated control crop plant. The term “crop yield” refers to the return on crop material, in terms of both quantity and quality, obtained after harvesting a crop plant. An “increase in crop yield” refers to an increase in crop yield relative to an untreated control crop plant.

The term “biologically effective amount” refers to the amount of a biologically active compound (e.g., a compound of Formula 1) sufficient to produce the desired biological effect when applied to (i.e. contacted with) an invertebrate pest to be controlled or its environment, or to a plant, the seed from which the plant is grown, or the locus of the plant (e.g., growth medium) to protect the plant from injury by the invertebrate pest or for other desired effect (e.g., increasing plant vigor).

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers.

“Alkoxy” includes, for example, methoxy, ethoxy, n-propyloxy, isopropyloxy and the different butoxy, pentoxy and hexyloxy isomers. “Alkylthio” includes branched or straight-chain alkylthio moieties such as methylthio, ethylthio, and the different propylthio, butylthio, pentylthio and hexylthio isomers.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—.

The chemical abbreviations S(O) and S(═O) as used herein represent a sulfinyl moiety. The chemical abbreviations SO₂, S(O)₂ and S(═O)₂ as used herein represent a sulfonyl moiety. The chemical abbreviations C(O) and C(═O) as used herein represent a carbonyl moiety. The chemical abbreviations CO₂, C(O)O and C(═O)O as used herein represent an oxycarbonyl moiety.

A dashed line in a structure fragment denotes the attachment point of the fragment to the remainder of the molecule. For example, when the variable Q in Formula 1 is defined as Q-1, the dashed line in the structure of Q-1 means that Q-1 is attached to the remainder of the structure of Formula 1 at that position, as shown below. Moreover, compounds of Formula 1 wherein the Q is selected from pyridine, pyridazine, pyrimidine, and pyrazine said pyridine, pyridazine, pyrimidine, and pyrazine is attached to the pyrazole ring of Formula 1 in such a way that a nitrogen ring atom of the pyridine, pyridazine, pyrimidine, or pyrazine is adjacent to the point of attachment.

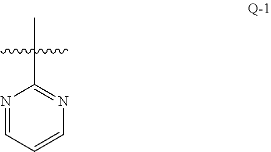 [SMILES: *c1ncccn1]

As noted above, Q can be pyridine, pyridazine, pyrimidine, pyrazine, or thiazole wherein the pyridine, pyridazine, pyrimidine, or pyrazine is optionally substituted with one or more substituents selected from a group of substituents as defined in the Summary of Invention. Non-limiting examples of variable Q are shown in Exhibit 1.

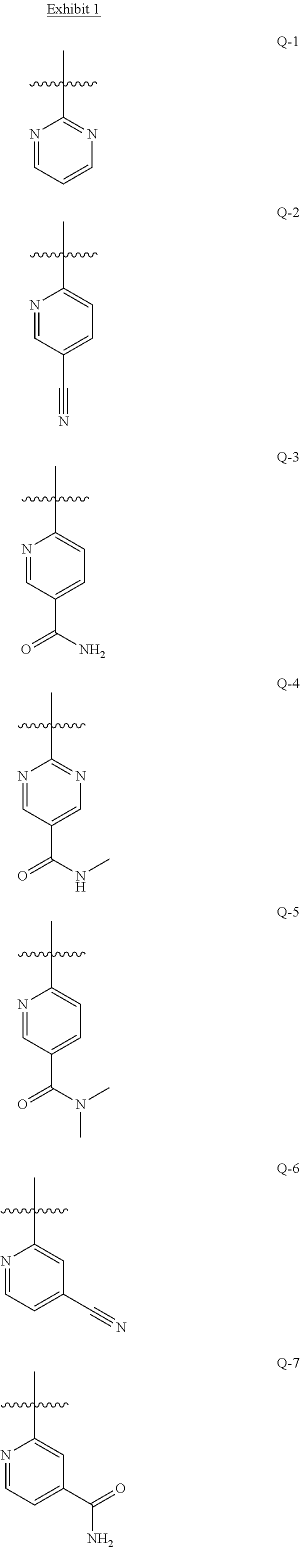

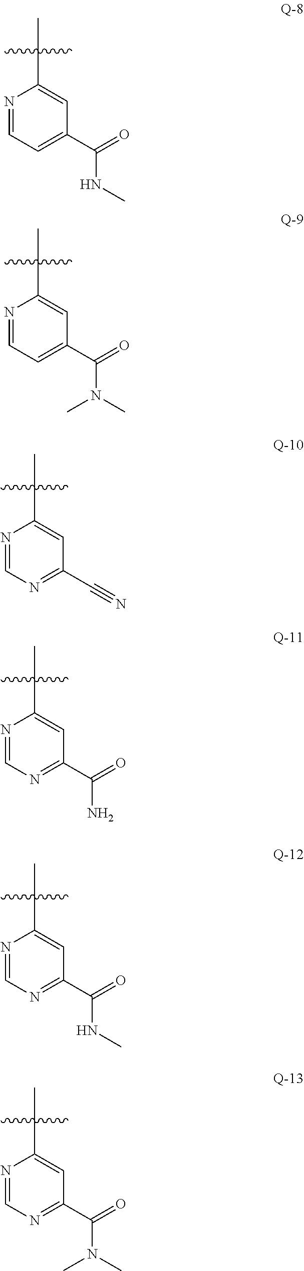 [SMILES: *c1cc(C(=O)NC)ccn1 ; *c1cc(C(=O)N(C)C)ccn1 ; *c1cc(C#N)ncn1 ; *c1cc(C(N)=O)ncn1 ; *c1cc(C(=O)NC)ncn1 ; *c1cc(C(=O)N(C)C)ncn1]

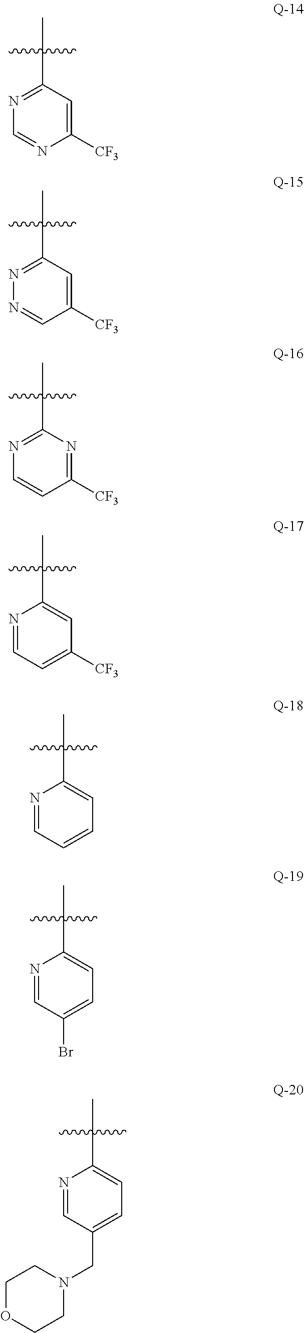

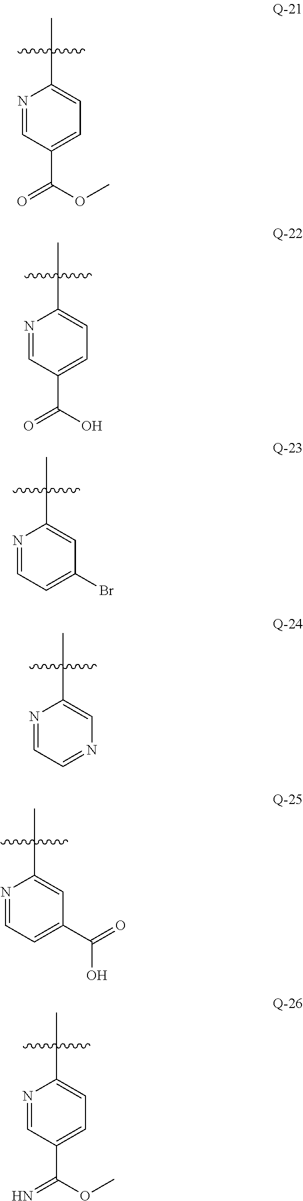 [SMILES: *c1ccc(C(=O)OC)cn1 ; *c1ccc(C(=O)O)cn1 ; *c1cc(Br)ccn1 ; *c1cnccn1 ; *c1cc(C(=O)O)ccn1 ; *c1ccc(C(=N)OC)cn1]

total number of carbon atoms in a substituent group is indicated by the “C₁-C₃” prefix. For example, C₁-C₄ alkyl designates methyl, ethyl, and the various propyl and butyl isomers.

When a compound is substituted with a substituent bearing a subscript that indicates the number of said substituents can exceed 1, said substituents (when they exceed 1) are independently selected from the group of defined substituents. Further, when the subscript indicates a range, e.g. (R)ᵢ₋ⱼ, then the number of substituents may be selected from the integers between i and j inclusive. When a group contains a substituent which can be hydrogen, then when this substituent is taken as hydrogen, it is recognized that this is equivalent to said group being unsubstituted. When one or more positions on a group are said to be “not substituted” or “unsubstituted”, then hydrogen atoms are attached to take up any free valency.

When a substituent is a 5- or 6-membered nitrogen-containing heterocyclic ring, it may be attached to the remainder of Formula 1 though any available carbon or nitrogen ring atom, unless otherwise described.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. For a comprehensive discussion of all aspects of stereoisomerism, see Ernest L. Eliel and Samuel H. Wilen, Stereochemistry of Organic Compounds, John Wiley & Sons, 1994.

The compounds of the disclosure may be present as a mixture of stereoisomers or individual stereoisomers. For example, two possible enantiomers of Formula 1 are depicted as Formula 1a and Formula 1a′ involving a chiral carbon center identified with an asterisk (*).

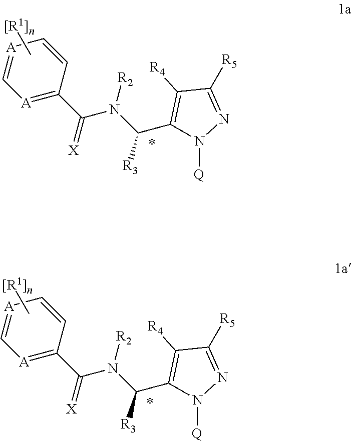

Molecular depictions drawn herein follow standard conventions for depicting stereochemistry. To indicate stereo configuration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the broad end of the wedge is attached to the atom further away from the viewer.

The compounds of the disclosure can exist as stereoisomers due to the possible chiral carbon atoms present in Formula 1. Thus, this disclosure comprises the individual stereoisomers of the compounds of Formula 1, as well as mixtures of stereoisomers of the compounds of Formula 1.

Compounds of Formula 1 may comprise additional chiral centers. This disclosure comprises racemic mixtures as well as enriched and essentially pure stereo configurations at these additional chiral centers.

Compounds of this disclosure can exist as one or more conformational isomers due to restricted rotation about any bonds in Formula 1. This disclosure comprises mixtures of conformational isomers. In addition, this disclosure includes compounds that are enriched in one conformer relative to others.

The more biologically active enantiomer is believed to be Formula 1a (the R-enantiomer of Formula 1.

This disclosure comprises racemic mixtures of equal amounts of the enantiomers of Formulae 1a (the S-enantiomer of Formula 1) and 1a′ (the R-enantiomer of Formula 1). In addition, this disclosure includes mixtures that are enriched in the Formula 1a enantiomer compared to the racemic mixture of Formulae 1a and 1a′. This disclosure also comprises the essentially pure enantiomer of Formula 1a.

An embodiment of this disclosure comprises mixtures of stereoisomers of the compounds of Formula 1a and Formula 1a′, wherein the ratio of 1a to 1a′ is at least 75:25 (a 50% enantiomeric excess).

An embodiment of this disclosure comprises mixtures of stereoisomers of the compounds of Formula 1a and Formula 1a′, wherein the ratio of 1a to 1a′ is at least 90:10 (an 80% enantiomeric excess of 1a).

An embodiment of this disclosure comprises mixtures of stereoisomers of the compounds of Formula 1a and Formula 1a′, wherein the ratio of 1a to 1a′ is at least 95:5 (a 90% enantiomeric excess of 1a).

An embodiment of this disclosure comprises mixtures of stereoisomers of the compounds of Formula 1a and Formula 1a′, wherein the ratio of 1a to 1a′ is at least 98:2 (a 96% enantiomeric excess of 1a).

An embodiment of this disclosure comprises mixtures of stereoisomers of the compounds of Formula 1a and Formula 1a′, wherein the ratio of 1a to 1a′ is at least 99:1 (a 98% enantiomeric excess of 1a).

An embodiment of this disclosure comprises mixtures of stereoisomers of the compounds of Formula 1a and Formula 1a′, wherein the ratio of 1a to 1a′ is essentially 100:0.

An embodiment of this disclosure comprises the compounds of Formula 1a.

Compounds selected from Formula 1 typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures. Compounds of this invention may exist as one or more crystalline polymorphs. This invention comprises both individual polymorphs and mixtures of polymorphs, including mixtures enriched in one polymorph relative to others. For a comprehensive discussion of polymorphism see R. Hilfiker, Ed., Polymorphism In the Pharmaceutical Industry, Wiley-VCH, Weinheim, 2006.

Embodiments of the present invention as described in the Summary include those described below. In the following Embodiments, Formula 1 includes stereoisomers, N-oxides and salts thereof.

Embodiment 1: A compound of Formula 1 wherein X is O.
Embodiment 1a: A compound of Formula 1 wherein X is S.
Embodiment 2: A compound of Formula 1, Embodiment 1 or Embodiment 1a wherein A is CR¹.
Embodiment 2a: A compound of Formula 1, Embodiment 1 or Embodiment 1a wherein A is CH.
Embodiment 2b: A compound of Formula 1, Embodiment 1 or Embodiment 1a wherein A is N.
Embodiment 3: A compound of Formula 1 or any of the foregoing Embodiments wherein R¹ is independently H, amino, cyano, halogen, nitro, C(S)NH₂; or R¹ is independently C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is independently phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is independently C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is independently a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸.
Embodiment 3a: A compound of Formula 1 or any of the foregoing Embodiments wherein R¹ is independently amino, cyano, halogen, nitro, C(S)NH₂; or R¹ is independently C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is independently phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is independently C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is independently a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸.
Embodiment 3b: A compound of Formula 1 or any of the foregoing Embodiments wherein R¹ is independently cyano, halogen, nitro, C(S)NH₂; or R¹ is independently C₁-C₃ alkyl, C₂-C₄ alkenyl, C₂-C₄ alkenyl, C₂-C₄ alkynyl, C₃-C₆ cycloalkyl, or C₃-C₇ cycloalkylalkyl wherein each alkyl, alkenyl, alkynyl, cycloalkyl, or cycloalkylalkyl is optionally substituted with one or more R⁶; or R¹ is independently C₃-C₉-trialkylsilyl, C(O)C₁-C₆, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is independently a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, or S(O)ₚR⁷.
Embodiment 3c: A compound of Formula 1 or any of the foregoing Embodiments wherein R¹ is halogen, or R¹ is C₁-C₃ alkyl, C₂-C₄ alkenyl, C₂-C₄ alkynyl, or C₃-C₆ cycloalkyl, wherein each alkyl, alkenyl, alkynyl or cycloalkyl is optionally substituted with one or more R⁶;

or R¹ is C₃-C₉-trialkylsilyl, C(O)C₁-C₆, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, or C(O)OC₁-C₆.

Embodiment 3d: A compound of Formula 1 or any of the foregoing Embodiments wherein R¹ is halogen, or R¹ is C₁-C₃ alkyl, C₂-C₄ alkenyl, C₂-C₄ alkynyl, or, wherein each alkyl, alkenyl, or alkynyl, is optionally substituted with one or more R⁶; or R¹ is C(O)C₁-C₆, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, or OS(O)₂R⁹.
Embodiment 3e: A compound of Formula 1 or any of the foregoing Embodiments wherein R¹ is halogen, or R¹ is C₁-C₃ alkyl, C₂-C₄ alkenyl, or C₂-C₄ alkynyl, wherein each alkyl, alkenyl, or alkynyl is optionally substituted with one or more R⁶; or R¹ is C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, or OS(O)₂R⁹.
Embodiment 3f: A compound of Formula 1 or any of the foregoing Embodiments wherein R¹ is halogen, or R¹ is C₁-C₃ alkyl, or C₂-C₄ alkenyl, wherein each alkyl, or alkenyl, is optionally substituted with one or more R⁶; or R¹ is CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, or OS(O)₂R⁹.
Embodiment 3g: A compound of Formula 1 or any of the foregoing Embodiments wherein R¹ is halogen, or R¹ is C₁-C₃ alkyl, wherein each alkyl, is optionally substituted with one or more R⁶; or R¹ is OR⁷, SCF₃ or OS(O)₂R⁹.
Embodiment 3h: A compound of Formula 1 or any of the foregoing Embodiments wherein R¹ is Cl, F, or Br, or R¹ is C₁-C₃ alkyl, wherein each alkyl, is optionally substituted with one or more R⁶; or R¹ is OR⁷, SCF₃, or OS(O)₂R⁹.
Embodiment 3i: A compound of Formula 1 or any of the foregoing Embodiments wherein R¹ is Cl, F, or Br, or R¹ is C₁-C₃ alkyl, wherein each alkyl, is optionally substituted with one or more R⁶; or R¹ is OMe, SCF₃, or OS(O)₂R⁹ Embodiment 3j: A compound of Formula 1 or any of the foregoing Embodiments wherein R¹ is Cl, F, or Br, or R¹ is Me optionally substituted with one or more R⁶; or R¹ is OMe, SCF₃, or OS(O)₂R⁹.
Embodiment 3k: A compound of Formula 1 or any of the foregoing Embodiments wherein R¹ is Cl, F, or Br, or R¹ is C₁-C₃ alkyl, wherein each alkyl, is optionally substituted with one or more R⁶;
Embodiment 3l: A compound of Formula 1 or any of the foregoing Embodiments wherein R¹ is Cl, or R¹ is Me optionally substituted with one or more R⁶.
Embodiment 3m: A compound of Formula 1 or any of the foregoing Embodiments wherein n is 2 and each R¹ is Cl, or each R¹ is CF₃, or one R₁ is Cl and one R¹ is CF₃.
Embodiment 3n: A compound of Formula 1 or any of the foregoing Embodiments wherein n is 2 and each R¹ is located at the 3 and 5 position of the phenyl ring.
Embodiment 4: A compound of Formula 1 or any one of the foregoing Embodiments wherein R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or

R² is C(O)R⁷, C(O)OR⁷, NR⁷R⁸, OR⁷, S(O)ₚR⁹ or SO₂NR⁷R⁸;

Embodiment 4a: A compound of Formula 1 or any one of the foregoing Embodiments wherein R² is hydrogen, C₁-C₄ alkyl, or C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶;
Embodiment 4b: A compound of Formula 1 or any one of the foregoing Embodiments wherein R² is hydrogen, or C₁-C₄ alkyl, wherein each alkyl is optionally substituted with one R⁶; or R² is C(O)R⁷ or C(O)OR⁷.
Embodiment 4c: A compound of Formula 1 or any one of the foregoing Embodiments wherein R² is hydrogen, or C₁-C₄ alkyl, wherein each alkyl is optionally substituted with one R⁶; or R² is C(O)R⁷.
Embodiment 4d: A compound of Formula 1 or any one of the foregoing Embodiments wherein R² is H, or C₁-C₄ alkyl, wherein each alkyl is optionally substituted with one R⁶.
Embodiment 4e: A compound of Formula 1 or any one of the foregoing Embodiments wherein R² is H, or C₁-C₃ alkyl, wherein each alkyl is optionally substituted with one R⁶.
Embodiment 4f: A compound of Formula 1 or any one of the foregoing Embodiments wherein R² is H, or C₁-C₃ alkyl, or CH₂c-Pr.
Embodiment 4f: A compound of Formula 1 or any one of the foregoing Embodiments wherein R² is H, or Me.
Embodiment 5: A compound of Formula 1 or any one of the foregoing Embodiments wherein R³ is C₁-C₃ alkyl or C₁-C₃ haloalkyl.
Embodiment 5a: A compound of Formula 1 or any one of the foregoing Embodiments wherein R³ is C₁-C₃ alkyl.
Embodiment 5b: A compound of Formula 1 or any one of the foregoing Embodiments wherein R³ is Me.
Embodiment 6: A compound of Formula 1 or any one of the foregoing Embodiments wherein R⁴ is hydrogen, cyano, halogen, nitro, C(S)NH₂, SCN, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R⁴ is C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸.
Embodiment 6a: A compound of Formula 1 or any one of the foregoing Embodiments wherein R⁴ is hydrogen, cyano, halogen, nitro, or C₁-C₃ alkyl wherein the alkyl is optionally substituted with one or more R⁶, or R⁴ is NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸.
Embodiment 6b: A compound of Formula 1 or any one of the foregoing Embodiments wherein R⁴ is hydrogen, cyano, halogen, nitro, or C₁-C₃ alkyl wherein the alkyl is optionally substituted with one or more R⁶, or R⁴ is NR⁷R⁸, NR⁸C(O)R⁷, OR⁷, S(O)ₚR⁹, or SO₂NR⁷R⁸.
Embodiment 6b: A compound of Formula 1 or any one of the foregoing Embodiments wherein R⁴ is hydrogen, cyano, halogen, nitro, or C₁-C₃ alkyl wherein the alkyl is optionally substituted with one or more R⁶, or R⁴ is NR⁷R⁸, NR⁸C(O)R⁷, OR⁷, or S(O)ₚR⁹.
Embodiment 6c: A compound of Formula 1 or any one of the foregoing Embodiments wherein R⁴ is hydrogen, cyano, nitro, Cl, or C₁-C₃ alkyl wherein the alkyl is optionally substituted with one or more R⁶, or R⁴ is NR⁷R⁸, NR⁸C(O)R⁷, OR⁷, or S(O)ₚR⁹.
Embodiment 6d: A compound of Formula 1 or any one of the foregoing Embodiments wherein R⁴ is cyano, nitro, or SO₂Me.
Embodiment 7: A compound of Formula 1 or any one of the foregoing Embodiments wherein R⁵ is hydrogen, cyano, halogen, nitro, C(S)NH₂, SCN, C₁-C₆-alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R⁵ is C(O)R⁷, CR⁷(═NO)R⁸, C(O)OR⁷, C(O)NR⁷R⁸, NR⁷R⁸, NR⁸C(O)R⁷, NR⁸C(O)OR⁷, NR¹¹SO₂NR⁷R⁸, OR⁷, S(O)ₚR⁹, or SO₂NR⁷R⁸;
Embodiment 7a: A compound of Formula 1 or any one of the foregoing Embodiments wherein R⁵ is hydrogen, halogen, nitro, C₁-C₃-alkyl or C₃-C₆-cycloalkyl wherein each alkyl or cycloalkyl is optionally substituted with one or more R⁶; or R⁵ is OR⁷, NR⁷R⁸, NR⁸C(O)R⁷, NR⁸C(O)OR⁷, S(O)ₚR⁷, or SO₂NR⁷R⁸.
Embodiment 7b: A compound of Formula 1 or any one of the foregoing Embodiments wherein R⁵ is hydrogen, cyano, halogen, nitro, or C₁-C₃-alkyl or C₃-C₆-cycloalkyl wherein each alkyl is optionally substituted with one or more R⁶; or R⁵ is OR⁷, NR⁷R⁸, NR⁸C(O)R⁷, NR⁸C(O)OR⁷, S(O)ₚR⁷, or SO₂NR⁷R⁸.
Embodiment 7c: A compound of Formula 1 or any one of the foregoing Embodiments wherein R⁵ is hydrogen, cyano, halogen, nitro, or C₁-C₃-alkyl wherein each alkyl or cycloalkyl is optionally substituted with one or more R⁶; or R⁵ is OR⁷, NR⁷R⁸, NR⁸C(O)R⁷, NR⁸C(O)OR⁷, S(O)ₚR⁷, or SO₂NR⁷R⁸.
Embodiment 7d: A compound of Formula 1 or any one of the foregoing Embodiments wherein R⁵ hydrogen, cyano, halogen, nitro, or C₁-C₃-alkyl; or R⁵ is OR⁷, NR⁷R⁸, NR⁸C(O)R⁷, NR⁸C(O)OR⁷, S(O)ₚR⁷, or SO₂NR⁷R⁸.
Embodiment 7e: A compound of Formula 1 or any one of the foregoing Embodiments wherein R⁵ is hydrogen, halogen, or R⁵ is OR⁷, NR⁷R⁸, NR⁸C(O)R⁷, or NR⁸C(O)OR⁷.
Embodiment 7f: A compound of Formula 1 or any one of the foregoing Embodiments wherein R⁵ is hydrogen, cyano, Br, Cl, F, nitro, or C₁-C₃-alkyl; or R⁵ is OR⁷, NR⁷R⁸, NR⁸C(O)R⁷, NR⁸C(O)OR⁷, S(O)ₚR⁷, or SO₂NR⁷R⁸.
Embodiment 7g: A compound of Formula 1 or any one of the foregoing Embodiments wherein R⁵ is hydrogen, cyano, Br, Cl, F, nitro, or C₁-C₃-alkyl; or R⁵ is OR⁷, NR⁷R⁸, NR⁸C(O)R⁷, or NR⁸C(O)OR⁷.
Embodiment 7h: A compound of Formula 1 or any one of the foregoing Embodiments wherein R⁵ is hydrogen, cyano, Br, Cl, F, nitro, or Me; or R⁵ is OR⁷, NR⁷R⁸, NR⁸C(O)R⁷, or NR⁸C(O)OR⁷.
Embodiment 7i: A compound of Formula 1 or any one of the foregoing Embodiments wherein R⁵ is hydrogen, cyano, Br, Cl, F, nitro, or Me; or R⁵ is OMe, NH₂, NHMe, SMe, N(Me)₂, or NHC(O)Me.
Embodiment 7j: A compound of Formula 1 or any one of the foregoing Embodiments wherein R⁵ is hydrogen, cyano, Cl, nitro, or Me; or R⁵ is OMe, NH₂, NHMe, SMe, N(Me)₂, or NHC(O)Me.
Embodiment 8: A compound of Formula 1 or any one of the foregoing Embodiments wherein R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸, or S(═NR)OₚR⁷;
Embodiment 8a: A compound of Formula 1 or any one of the foregoing Embodiments wherein R⁶ is halogen, cyano, nitro, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, SF₅, S(O)ₚR⁹, or SO₂NR⁷R⁸.
Embodiment 8b: A compound of Formula 1 or any one of the foregoing Embodiments wherein R⁶ is halogen, cyano, nitro, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, or C(O)NR⁷R⁸.
Embodiment 8c: A compound of Formula 1 or any one of the foregoing Embodiments wherein R⁶ is halogen, cyano, nitro, C₁-C₃ alkyl, C₁-C₃ haloalkyl, C₁-C₃ alkoxy, C₁-C₃ haloalkoxy, or C₃-C₇ cycloalkyl.
Embodiment 8d: A compound of Formula 1 or any one of the foregoing Embodiments wherein R⁶ is halogen, cyano, C₁-C₃ alkyl, or C₁-C₃ haloalkyl.
Embodiment 8e: A compound of Formula 1 or any one of the foregoing Embodiments wherein R⁶ is F, cyano, C₁-C₃ alkyl, or C₁-C₃ haloalkyl.
Embodiment 9: A compound of Formula 1 or any one of the foregoing Embodiments wherein R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R⁶; or

R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶.

Embodiment 9a: A compound of Formula 1 or any one of the foregoing Embodiments wherein R⁷ is hydrogen, C₁-C₃ alkyl, C₂-C₃ alkenyl, C₃-C₄ alkynyl, C₃-C₆ cycloalkyl, or C₅-C₇ cycloalkylalkyl, wherein each alkyl, alkenyl, alkynyl or cycloalkyl is optionally substituted with one or more R¹²; or

R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R¹².

Embodiment 10: A compound of Formula 1 or any one of the foregoing Embodiments wherein R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶; or

when R⁷ and R⁸ are on N, R⁷ and R⁸ can form a ring of 3 to 7 carbons, optionally including oxygen, S(O)p or NR⁷.

Embodiment 10a: A compound of Formula 1 or any one of the foregoing Embodiments wherein R⁸ is hydrogen, or C₁-C₃, C₁-C₃ alkoxy, C₃-C₄ alkenyl, C₄-C₅ alkenyloxy, C₃-C₅ alkynyl, or C₄-C₅ alkynyloxy, wherein each alkyl, alkoxy, alkenyl, alkenyloxy, alkynyl, or alkynyloxy is optionally substituted with one or more R⁶.
Embodiment 11: A compound of Formula 1 or any one of the foregoing Embodiments wherein R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or

R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;

Embodiment 11a: A compound of Formula 1 or any one of the foregoing Embodiments wherein R⁹ is C₁-C₃ alkyl, C₃-C₅ alkenyl, C₃-C₅ alkynyl, or C₄-C₆ cycloalkyl, wherein each alkyl, alkenyl, alkynyl or cycloalkyl is optionally substituted with one or more R⁶; or

R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;

Embodiment 11b: A compound of Formula 1 or any one of the foregoing Embodiments wherein R⁹ is C₁-C₃ alkyl, C₃-C₅ alkenyl, C₃-C₅ alkynyl, or C₄-C₆ cycloalkyl, wherein each alkyl, alkenyl, alkynyl or cycloalkyl is optionally substituted with one or more R⁶.
Embodiment 12: A compound of Formula 1 or any one of the foregoing Embodiments wherein R¹⁰ is H, C₁-C₆ alkyl, C₁-C₆-haloalkyl, C₃-C₆-cycloalkyl, C₃-C₆-cycloalkyl-C₁-C₃-alkyl, or C₁-C₃-alkoxy-C₁-C₃-alkyl.
Embodiment 12a: A compound of Formula 1 or any one of the foregoing Embodiments wherein R¹⁰ is H, C₁-C₆ alkyl, C₁-C₆-haloalkyl, C₃-C₆-cycloalkyl, C₃-C₆-cycloalkyl-C₁-C₃-alkyl.
Embodiment 12b: A compound of Formula 1 or any one of the foregoing Embodiments wherein R¹⁰ is H, C₁-C₆ alkyl, C₁-C₆-haloalkyl, C₃-C₆-cycloalkyl.
Embodiment 12c: A compound of Formula 1 or any one of the foregoing Embodiments wherein R¹⁰ is H, C₁-C₆ alkyl, C₁-C₆-haloalkyl.
Embodiment 13: A compound of Formula 1 or any one of the foregoing Embodiments wherein R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷.
Embodiment 13a: A compound of Formula 1 or any one of the foregoing Embodiments wherein R¹¹ is hydrogen, C₁-C₃ alkyl, C₃-C₅ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷.
Embodiment 14: A compound of Formula 1 or any one of the foregoing Embodiments wherein R¹² is halogen, hydroxy, cyano, nitro, SF₅, C(O)NH₂, C(S)NH₂, C₁-C₃ alkyl, C₁-C₃ alkoxy, C₂-C₆ alkenyl, C₂-C₆ alkynyl, C₃-C₇ cycloalkyl, C₄-C₈ cycloalkylalkyl, wherein each alkyl, alkoxy, alkenyl, alkynyl, cycloalkyl or cycloalkylalkyl is optionally substituted with halogen or cyano.
Embodiment 14a: A compound of Formula 1 or any one of the foregoing Embodiments wherein R¹² is halogen, hydroxy, cyano, nitro, SF₅, C(O)NH₂, C(S)NH₂, C₁-C₃ alkyl, C₁-C₃ alkoxy, C₂-C₆ alkenyl, C₂-C₆ alkynyl, wherein each alkyl, alkoxy, alkenyl, alkynyl is optionally substituted with halogen or cyano.
Embodiment 14b: A compound of Formula 1 or any one of the foregoing Embodiments wherein R¹² is halogen, hydroxy, cyano, nitro, SF₅, C(O)NH₂, C(S)NH₂, C₁-C₃ alkyl, wherein each alkyl, is optionally substituted with halogen or cyano.
Embodiment 15: A compound of Formula 1 or any one of the foregoing Embodiments wherein Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
Embodiment 15a: A compound of Formula 1 or any one of the foregoing Embodiments wherein Q pyridine, pyridazine, pyrimidine, or pyrazine, wherein the pyridine, pyridazine, pyrimidine, or pyrazine is optionally substituted with one to three groups, these groups being chosen from amino, cyano, halogen, nitro, C₁-C₃ alkyl, C₅-C₇ cycloalkylalkyl, C₃-C₅ alkenyl, C₂-C₆ alkynyl, C₃-C₇ cycloalkyl, or cycloalkylalkyl, wherein each alkyl, alkoxy, cycloalkyloxy, alkoxyalkoxy, alkoxyalkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
Embodiment 15b: A compound of Formula 1 or any one of the foregoing Embodiments wherein Q is pyridine, pyridazine, or pyrimidine, wherein the pyridine, pyridazine, or-pyrimidine is optionally substituted with one to three groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.

Embodiments 15c: A compound of Formula 1 or any one of the foregoing Embodiments wherein Q is selected from Q-1, Q-2, Q-3, Q-4, Q-5, Q-6, Q-7, Q8, Q-9, Q-10, Q-11, Q-12, Q-13, Q-14, Q-15, Q-16, Q-17, Q-18, Q-19, Q-20, Q-21, Q-22, Q-23, Q-24, Q-25, and Q-26.

Embodiments 15d: A compound of Formula 1 or any one of the foregoing Embodiments wherein Q is selected from Q-1, Q-2, Q-6, Q-7, Q-11, Q-13, Q-14, Q-18, Q-19, Q-20, Q-21, Q-22, Q-23, Q-24, and Q-25.

Embodiments 15e: A compound of Formula 1 or any one of the foregoing Embodiments wherein Q is selected from Q-1, Q-2, Q-13, Q-14, Q-19, and Q-24.

Embodiments 15f: A compound of Formula 1 or any one of the foregoing Embodiments wherein Q is selected from Q-1, and Q-2.

Embodiments 15g: A compound of Formula 1 or any one of the foregoing Embodiments wherein Q is Q-2.

Embodiment 16: A compound of Formula 1 or any one of the foregoing Embodiments wherein n is 1 or 2.
Embodiment 16a: A compound of Formula 1 or any one of the foregoing Embodiments wherein n is 2.
Embodiment 17: A compound of Formula 1 or any one of the foregoing Embodiments wherein p is 0 or 1.
Embodiment 17a: A compound of Formula 1 or any one of the foregoing Embodiments wherein p is 0.

Embodiments of this invention, including Embodiments 1-17a above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this disclosure, including Embodiments 1-17a above as well as any other embodiments described herein, and any combination thereof, pertain to the compositions and methods of the present invention.

In some embodiments, when R¹ is other than H, R¹ is not located adjacent to C(X).

In some embodiments, R⁴ and R⁵ are not both hydrogen.

In some embodiments, when R¹ is other than H, R¹ is not located adjacent to C(X), and R⁴ and R⁵ are not both hydrogen.

Embodiment X. A method for controlling and combating an invertebrate pest comprising contacting the invertebrate pest or its environment with a biologically effective amount of a compound of Formula 1.

Embodiment X1. The method of Claim X wherein the environment is soil or plant foliage.

Embodiments of this disclosure, including Embodiments 1-X1 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this disclosure, including Embodiments 1-X1 above as well as any other embodiments described herein, and any combination thereof, pertain to the compositions and methods of the present disclosure.

Combinations of Embodiments 1-17a are illustrated by:

Embodiment A1. The compound of Formula 1 wherein
X is O;
n is 1, 2, or 3;
p is 0, 1, or 2;
A is CH;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸;
R³ is C₁-C₃ alkyl or C₁-C₃ haloalkyl;
R⁴ is hydrogen, cyano, halogen, nitro, C(S)NH₂, SCN, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸;
R⁵ is hydrogen, cyano, halogen, nitro, C(S)NH₂, SCN, C₁-C₆-alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁸, C(O)OR⁷, C(O)NR⁷R⁸, NR⁷R⁸, NR⁸C(O)R⁷, NR⁸C(O)OR⁷, NR¹¹SO₂NR⁷R⁸, OR⁷, S(O)ₚR⁹, or SO₂NR⁷R⁸;
R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸, or S(═NR)OₚR⁷;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R¹²; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R¹²;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
When R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
R¹² is halogen, hydroxy, cyano, nitro, SF₅, C(O)NH₂, C(S)NH₂, C₁-C₃ alkyl, C₁-C₃ alkoxy, C₂-C₆ alkenyl, C₂-C₆ alkynyl, C₃-C₇ cycloalkyl, C₄-C₈ cycloalkylalkyl, wherein each alkyl, alkoxy, alkenyl, alkynyl, cycloalkyl or cycloalkylalkyl is optionally substituted with halogen or cyano.
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A2. The compound of Formula 1 or Embodiment A1 wherein
X is O;
n is 1, 2, or 3;
p is 0, 1, or 2;
A is CH;
R¹ is halogen, or R¹ is C₁-C₃ alkyl, or C₂-C₄ alkenyl, wherein each alkyl, or alkenyl, is optionally substituted with one or more R⁶; or R¹ is CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, or OS(O)₂R⁹.
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more
R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸;
R³ is C₁-C₃ alkyl;
R⁴ is hydrogen, cyano, halogen, nitro, C(S)NH₂, SCN, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸;
R⁵ is hydrogen, cyano, halogen, nitro, C(S)NH₂, SCN, C₁-C₆-alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁸, C(O)OR⁷, C(O)NR⁷R⁸, NR⁷R⁸, NR⁸C(O)R⁷, NR⁸C(O)OR⁷, NR¹¹SO₂NR⁷R⁸, OR⁷, S(O)ₚR⁹, or SO₂NR⁷R⁸;
R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸, or S(═NR)OₚR⁷;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R¹²; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R¹²;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
When R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
R¹² is halogen, hydroxy, cyano, nitro, SF₅, C(O)NH₂, C(S)NH₂, C₁-C₃ alkyl, C₁-C₃ alkoxy, C₂-C₆ alkenyl, C₄-C₈ cycloalkylalkyl, wherein each alkyl, alkoxy, alkenyl, alkynyl, cycloalkyl or cycloalkylalkyl is optionally substituted with halogen or cyano;
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A3. The compound of Formula 1 or Embodiment A1 wherein
X is O;
n is 1, 2, or 3;
p is 0, 1, or 2;
A is CH;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸;
R³ is C₁-C₃ alkyl;
R⁴ is hydrogen, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸;
R⁵ is hydrogen, cyano, halogen, nitro, C₁-C₆-alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁸, C(O)OR⁷, C(O)NR⁷R⁸, NR⁷R⁸, NR⁸C(O)R⁷, NR⁸C(O)OR⁷, NR¹¹SO₂NR⁷R⁸, OR⁷, S(O)ₚR⁹, or SO₂NR⁷R⁸;
R⁶ is halogen, cyano, nitro, SF₅C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R¹²; or
R is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R¹²;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
When R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
R¹² is halogen, hydroxy, cyano, nitro, SF₅, C(O)NH₂, C(S)NH₂, C₁-C₃ alkyl, wherein each alkyl is optionally substituted with halogen or cyano.
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A4. The compound of Formula 1 or Embodiment A1 wherein
X is O;
n is 1, 2, or 3;
p is 0, 1, or 2;
A is CH;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸;
R³ is C₁-C₃ alkyl;
R⁴ is hydrogen, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶;
R⁵ is hydrogen, cyano, halogen, nitro, C₁-C₆-alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R⁵ is C(O)R⁷, CR⁷(═NO)R⁸, C(O)OR⁷, C(O)NR⁷R⁸, NR⁷R⁸, NR⁸C(O)R⁷, NR⁸C(O)OR⁷, OR⁷, S(O)ₚR⁹, or SO₂NR⁷R⁸;
R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R⁶; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
When R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A5. The compound of Formula 1 or Embodiment A1 wherein
X is O;
n is 1, 2, or 3;
p is 0, 1, or 2;
A is CH;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸;
R³ is C₁-C₃ alkyl;
R⁴ is hydrogen, cyano, halogen, nitro, C₁-C₆ alkyl, wherein each alkyl is optionally substituted with one or more R⁶;
R⁵ is hydrogen, cyano, halogen, nitro, C₁-C₆-alkyl, C₂-C₆ alkenyl, C₂-C₆ alkynyl, C₃-C₇ cycloalkyl, wherein each alkyl, alkenyl, alkynyl, cycloalkyl or cycloalkylalkyl is optionally substituted with one or more R⁶; or R⁵ is NR⁷R⁸, NR⁸C(O)R⁷, NR⁸C(O)OR⁷, OR⁷, S(O)ₚR⁹, or SO₂NR⁷R⁸;
R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, or SO₂NR⁷R⁸;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R⁶; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶.

R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
When R⁷ and R⁸ are on N, R⁷ and R⁸ can form a ring of 3 to 7 carbons, optionally including oxygen, S(O)ₚ or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷; Q is pyridine, pyridazine, pyrimidine, pyrazine, or thiazole wherein the pyridine, pyridazine,
pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A6. The compound of Formula 1 or Embodiment A1 wherein
X is O;
n is 1, 2, or 3;
p is 0, 1, or 2;
A is CH;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶
R³ is Me;
R⁴ is hydrogen, cyano, halogen, nitro, C₁-C₃ alkyl, wherein each alkyl is optionally substituted with one or more R⁶;
R⁵ is hydrogen, cyano, halogen, nitro; or R⁵ is NR⁷R⁸, NR⁸C(O)R⁷, OR⁷, S(O)ₚR⁹, or SO₂NR⁷R⁸;
R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R⁶; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
when R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A7. The compound of Formula 1 or Embodiment A1 wherein
X is O;
n is 1, or 2;
p is 0, 1, or 2;
A is CH;
R¹ is halogen; or R¹ is C₁-C₆ alkyl, wherein each alkyl is optionally substituted with one or more R⁶;
R² is hydrogen, or C₁-C₆ alkyl.
R³ is Me;
R⁴ is cyano, or nitro;
R⁵ is hydrogen, cyano, halogen, nitro; or R⁵ is NR⁷R⁸, NR⁸C(O)R⁷, OR⁷, S(O)ₚR⁹, or SO₂NR⁷R⁸;
R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R⁶; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
when R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷; and Q is selected from Q-1, Q-2, Q-3, Q-4, Q-5, Q-6, Q-7, Q-8, Q-9, Q-10, Q-11, Q-12, Q-13, Q-14, Q-15, Q-16, Q-17, Q-18, Q-19, Q-20, Q-21, Q-22, Q-23, Q-24, Q-25, and Q-26.
   Embodiment A8. The compound of Formula 1 or Embodiment A1, wherein
A is CH;
N is 1, or 2;
R¹ is Cl, or F, or R¹ is Me optionally substituted with one or more R⁶.
R² is hydrogen, CH2c-Pr, or Me;
R³ is Me;
R⁴ is hydrogen, cyano, halogen, nitro, or C₁-C₃ alkyl wherein the alkyl is optionally substituted with one or more R⁶, or R⁴ is NR⁷R⁸, NR⁸C(O)R⁷, OR⁷, or S(O)ₚR⁹;
R⁵ is hydrogen, cyano, Br, Cl, F, nitro, or C₁-C₃-alkyl; or R⁵ is OR⁷, NR⁷R⁸, NR⁸C(O)R⁷, or NR⁸C(O)OR⁷, and
Q is Q-1, Q-2, Q-6, Q-7, Q-11, Q-13, Q-14, Q-18, Q-19, Q-20, Q-21, Q-22, Q-23, Q-24, or Q-25.
   Embodiment A9. The compound of Formula lor Embodiment 1, wherein
X is O;
A is CH;
n is 2;
R¹ is Cl, or F, or R¹ is Me optionally substituted with one or more R⁶.
R² is hydrogen, CH2c-Pr, or Me;
R³ is Me;
R⁴ is hydrogen, cyano, halogen, nitro, or C₁-C₃ alkyl wherein the alkyl is optionally substituted with one or more R⁶, or R⁴ is NR⁷R⁸, NR⁸C(O)R⁷, OR⁷, or S(O)ₚR⁹;
R⁵ is hydrogen, cyano, Br, Cl, F, nitro, or C₁-C₃-alkyl; or R⁵ is OR⁷, NR⁷R⁸, NR⁸C(O)R⁷, or NR⁸C(O)OR⁷, and
Q is Q-1, Q-2, Q, Q-13, Q-14, Q-19, or Q-24.
   Embodiment A10. The compound of Formula 1 wherein
X is O;
n is 1, 2, or 3;
p is 0, 1, or 2;
A is CR¹;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸;
R³ is C₁-C₃ alkyl or C₁-C₃ haloalkyl;
R⁴ is hydrogen, cyano, halogen, nitro, C(S)NH₂, SCN, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸;
R⁵ is hydrogen, cyano, halogen, nitro, C(S)NH₂, SCN, C₁-C₆-alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁸, C(O)OR⁷, C(O)NR⁷R⁸, NR⁷R⁸, NR⁸C(O)R⁷, NR⁸C(O)OR⁷, NR¹¹SO₂NR⁷R⁸, OR⁷, S(O)ₚR⁹, or SO₂NR⁷R⁸;
R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸, or S(═NR)OₚR⁷;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R¹²; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R¹²;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
When R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
R¹² is halogen, hydroxy, cyano, nitro, SF₅, C(O)NH₂, C(S)NH₂, C₁-C₃ alkyl, C₁-C₃ alkoxy, C₂-C₆ alkenyl, C₂-C₆ alkynyl, C₃-C₇ cycloalkyl, C₄-C₈ cycloalkylalkyl, wherein each alkyl, alkoxy, alkenyl, alkynyl, cycloalkyl or cycloalkylalkyl is optionally substituted with halogen or cyano.
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A11. The compound of Formula 1 or Embodiment A10 wherein
X is O;
n is 1, 2, or 3;
p is 0, 1, or 2;
A is CR¹;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸;
R³ is C₁-C₃ alkyl;
R⁴ is hydrogen, cyano, halogen, nitro, C(S)NH₂, SCN, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸;
R⁵ is hydrogen, cyano, halogen, nitro, C(S)NH₂, SCN, C₁-C₆-alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁸, C(O)OR⁷, C(O)NR⁷R⁸, NR⁷R⁸, NR⁸C(O)R⁷, NR⁸C(O)OR⁷, NR¹¹SO₂NR⁷R⁸, OR⁷, S(O)ₚR⁹, or SO₂NR⁷R⁸;
R⁶ is halogen, cyano, nitro, SF₅,C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸, or S(═NR)OR⁷;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R¹²; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R¹²;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
When R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
R¹² is halogen, hydroxy, cyano, nitro, SF₅, C(O)NH₂, C(S)NH₂, C₁-C₃ alkyl, C₁-C₃ alkoxy, C₂-C₆ alkenyl, C₄-C₈ cycloalkylalkyl, wherein each alkyl, alkoxy, alkenyl, alkynyl, cycloalkyl or cycloalkylalkyl is optionally substituted with halogen or cyano;
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A12. The compound of Formula 1 or Embodiment A10 wherein
X is O;
n is 1, 2, or 3;
p is 0, 1, or 2;
A is CR¹;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸;
R³ is C₁-C₃ alkyl;
R⁴ is hydrogen, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸;
R⁵ is hydrogen, cyano, halogen, nitro, C₁-C₆-alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; C(O)R⁷, CR⁷(═NO)R⁸, C(O)OR⁷, C(O)NR⁷R⁸, NR⁷R⁸, NR⁸C(O)R⁷, NR⁸C(O)OR⁷, NR¹¹SO₂NR⁷R⁸, OR⁷, S(O)ₚR⁹, or SO₂NR⁷R⁸;
R⁶ is halogen, cyano, nitro, SF₅,C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R¹²; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R¹²;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
When R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
R¹² is halogen, hydroxy, cyano, nitro, SF₅, C(O)NH₂, C(S)NH₂, C₁-C₃ alkyl, wherein each alkyl is optionally substituted with halogen or cyano.
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A13. The compound of Formula 1 or Embodiment A10 wherein
X is O;
n is 1, 2, or 3;
p is 0, 1, or 2;
A is CR¹;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸;
R³ is C₁-C₃ alkyl;
R⁴ is hydrogen, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶;
R⁵ is hydrogen, cyano, halogen, nitro, C₁-C₆-alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R⁵ is C(O)R⁷, CR⁷(═NO)R⁸, C(O)OR⁷, C(O)NR⁷R⁸, NR⁷R⁸, NR⁸C(O)R⁷, NR⁸C(O)OR⁷, OR⁷, S(O)ₚR⁹, or SO₂NR⁷R⁸;
R⁶ is halogen, cyano, nitro, SF₅,C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R⁶; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
When R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A14. The compound of Formula 1 or Embodiment A10 wherein
X is O;
n is 1, 2, or 3;
p is 0, 1, or 2;
A is CR¹;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸;
R³ is C₁-C₃ alkyl;
R⁴ is hydrogen, cyano, halogen, nitro, C₁-C₆ alkyl, wherein each alkyl is optionally substituted with one or more R⁶;
R⁵ is hydrogen, cyano, halogen, nitro, C₁-C₆-alkyl, C₂-C₆ alkenyl, C₂-C₆ alkynyl, C₃-C₇ cycloalkyl, wherein each alkyl, alkenyl, alkynyl, cycloalkyl or cycloalkylalkyl is optionally substituted with one or more R⁶; or R⁵ is NR⁷R⁸, NR⁸C(O)R⁷, NR⁸C(O)OR⁷, OR⁷, S(O)ₚR⁹, or SO₂NR⁷R⁸;
R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, or SO₂NR⁷R⁸;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R⁶; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶.
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶; When R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A15. The compound of Formula 1 or Embodiment A10 wherein
X is O;
n is 1, 2, or 3;
p is 0, 1, or 2;
A is CR¹;
R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶
R³ is Me;
R⁴ is hydrogen, cyano, halogen, nitro, C₁-C₃ alkyl, wherein each alkyl is optionally substituted with one or more R⁶;
R⁵ is hydrogen, cyano, halogen, nitro; or R⁵ is NR⁷R⁸, NR⁸C(O)R⁷, OR⁷, S(O)ₚR⁹, or SO₂NR⁷R⁸;
R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸;
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R⁶; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
when R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.
   Embodiment A16. The compound of Formula 1 or Embodiment A10 wherein
X is O;
n is 1, or 2;
p is 0, 1, or 2;
A is CR¹;
R¹ is halogen; or R¹ is C₁-C₆ alkyl, wherein each alkyl is optionally substituted with one or more R⁶;
R² is hydrogen, or C₁-C₆ alkyl.
R³ is Me;
R⁴ is cyano, or nitro;
R⁵ is hydrogen, cyano, halogen, nitro; or R⁵ is NR⁷R⁸, NR⁸C(O)R⁷, OR⁷, S(O)ₚR⁹, or SO₂NR⁷R⁸;
R⁶ is halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸,
R⁷ is hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R⁶; or
R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶.

R⁸ is hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
when R⁷ and R⁸ are on N, R⁷ and R⁸ can form a ring of 3 to 7 carbons, optionally including oxygen, S(O)p or NR⁷;
R⁹ is C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶; or
R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
R¹⁰ is H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
R¹¹ is hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷.
   Embodiment A17. The compound of Formula lor Embodiment A10, wherein
X is O or S;
A is N or CR¹;
R¹ is Cl, F, or Br, or R¹ is Me optionally substituted with one or more R⁶; or R¹ is OMe, SF₃, or OS(O)₂R⁹.
R² is hydrogen, C₁-C₄ alkyl, or C₄-C₆ cycloalkylalkyl, wherein each alkyl or cycloalkylalkyl is optionally substituted with one R⁶; or
R² is C(O)R⁷ or C(O)OR⁷;
R³ is Me; and
R⁴ is hydrogen, cyano, halogen, nitro, or C₁-C₃ alkyl wherein the alkyl is optionally substituted with one or more R⁶.
   Embodiment A18. The compound of Formula 1 or Embodiment A10, wherein
X is O or S;
A is N or CR¹;
R¹ is Cl, F, or Br, or R¹ is Me optionally substituted with one or more R⁶; or R¹ is OMe, SF₃, or OS(O)₂R⁹.
R² is hydrogen, C₁-C₄ alkyl, or C₄-C₆ cycloalkylalkyl, wherein each alkyl or cycloalkylalkyl is optionally substituted with one R⁶; or
R² is C(O)R⁷ or C(O)OR⁷;
R³ is Me;
R⁴ is hydrogen, cyano, halogen, nitro, or C₁-C₃ alkyl wherein the alkyl is optionally substituted with one or more R⁶; and
Q is Q is Q-1, Q-2, Q-6, Q-7, Q-11, Q-13, Q-14, Q-18, Q-19, Q-20, Q-21, Q-22, Q-23, Q-24, or Q-25.
   Specific embodiments include compounds of Formula 1 selected from the group consisting of as depicted in Table A.

TABLE A

| Compound | | |
|---|---|---|
| (Cmp) | Structure | Chemical Name |
| Cmp 14 | 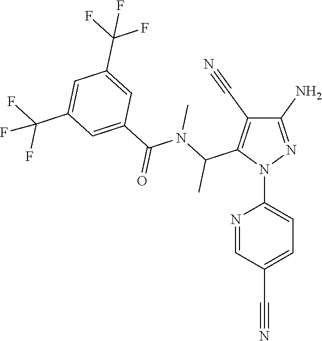 [SMILES: CC(c1c(C#N)c(N)nn1-c1ccc(C#N)cn1)N(C)C(=O)c1cc(C(F)(F)F)cc(C(F)(F)F)c1] | N-[1-[3-amino-4-cyano- 1-(5-cyano-2pyridinyl)- 1H-pyrazol-5-yl]ethyl]- N-methyl-3,5 bis(trifluoromethyl) Benzamide¹ |
| Cmp 12 | 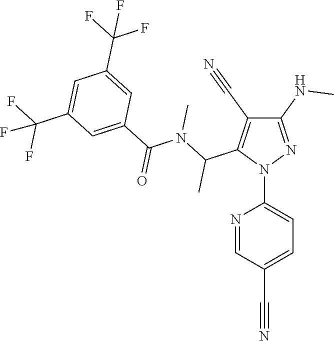 [SMILES: CNc1nn(-c2ccc(C#N)cn2)c(C(C)N(C)C(=O)c2cc(C(F)(F)F)cc(C(F)(F)F)c2)c1C#N] | N-[1-[4-cyano-1-(5- cyano-2-pyridinyl)-3- (methylamino)-1H- pyrazol-5-yl]ethyl]-N- methyl-3,5- bis(trifluoromethyl) benzamide¹ |
| Cmp 16 | 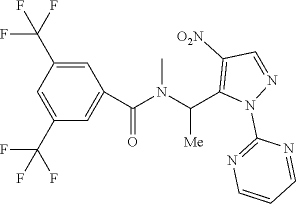 [SMILES: CC(c1c([N+](=O)[O-])cnn1-c1ncccn1)N(C)C(=O)c1cc(C(F)(F)F)cc(C(F)(F)F)c1] | N-methyl-N-(1-(4-nitro- 1-(pyrimidin-2-yl)-1H- pyrazol-5-yl)ethyl)-3,5- bis(trifluoromethyl) benzamide² |
| Cmp 29 | 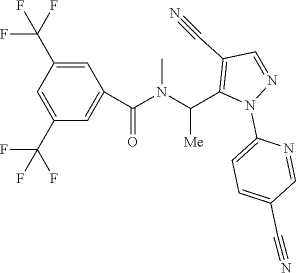 [SMILES: CC(c1c(C#N)cnn1-c1ccc(C#N)cn1)N(C)C(=O)c1cc(C(F)(F)F)cc(C(F)(F)F)c1] | N-(1-(4-cyano-1-(5- cyanopyridin-2-yl)-1H- pyrazol-5-yl)ethyl)-N- methyl-3,5- bis(trifluoromethyl) benzamide² |
| Cmp 41 | 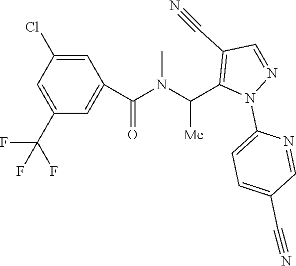 [SMILES: CC(c1c(C#N)cnn1-c1ccc(C#N)cn1)N(C)C(=O)c1cc(Cl)cc(C(F)(F)F)c1] | 3-chloro-N-(1-(4-cyano-1- (5-cyanopyridin-2-yl)-1H- pyrazol-5-yl)ethyl)-N- methyl-5- (trifluoromethyl) benzamide² |
| Cmp 44 | 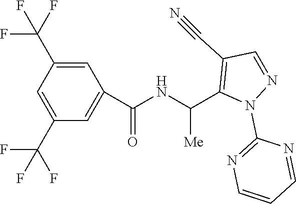 [SMILES: CC(NC(=O)c1cc(C(F)(F)F)cc(C(F)(F)F)c1)c1c(C#N)cnn1-c1ncccn1] | N-(1-(4-cyano-1- (pyrimidin-2-yl)-1H- pyrazol-5-yl)ethyl)-3,5- bis(trifluoromethyl) benzamide² |
| Cmp 26 | 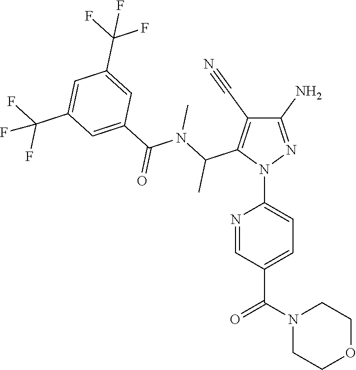 [SMILES: CC(c1c(C#N)c(N)nn1-c1ccc(C(=O)N2CCOCC2)cn1)N(C)C(=O)c1cc(C(F)(F)F)cc(C(F)(F)F)c1] | N-[1-[3-amino-4-cyano-1- [5-(4- morpholinylcarbonyl)-2- pyridinyl]-1H-pyrazol-5- ylethyl-N-methyl-3,5- bis(trifluoromethyl) benzamide¹ |
| Cmp 58 | 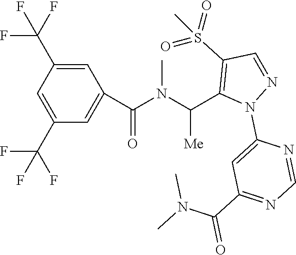 [SMILES: CC(c1c(S(C)(=O)=O)cnn1-c1cc(C(=O)N(C)C)ncn1)N(C)C(=O)c1cc(C(F)(F)F)cc(C(F)(F)F)c1] | N,N-dimethyl-6-(5-(1-(N- methyl-3,5- bis(trifluoromethyl) benzamido)ethyl)-4- (methylsulfonyl)-1H- pyrazol-1-yl)pyrimidine-4- carboxamide² |
| Cmp 82 | 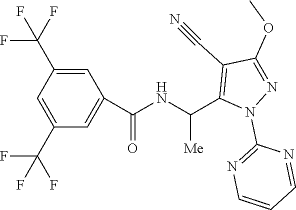 [SMILES: COc1nn(-c2ncccn2)c(C(C)NC(=O)c2cc(C(F)(F)F)cc(C(F)(F)F)c2)c1C#N] | N-(1-(4-cyano-3- methoxy-1-(pyrimidin-2- yl)-1H-pyrazol-5- yl)ethyl)-3,5- bis(trifluoromethyl) benzamide² |
| Cmp 109 | 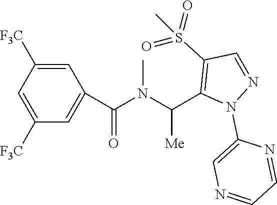 [SMILES: CC(c1c(S(C)(=O)=O)cnn1-c1cnccn1)N(C)C(=O)c1cc(C(F)(F)F)cc(C(F)(F)F)c1] | N-methyl-N-(1-(4- (methylsulfonyl)-1- (pyrazin-2-yl)-1H-pyrazol- 5-yl)ethyl)-3,5- bis(trifluoromethyl) benzamide² |
| Cmp 113 | 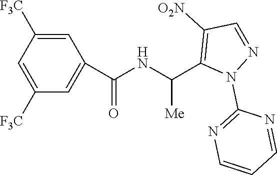 [SMILES: CC(NC(=O)c1cc(C(F)(F)F)cc(C(F)(F)F)c1)c1c([N+](=O)[O-])cnn1-c1ncccn1] | N-(1-(4-nitro-1-(pyrimidin- 2-yl)-1H-pyrazol-5- yl)ethyl)-3,5- bis(trifluoromethyl) benzamide² |
| Cmp 125 | 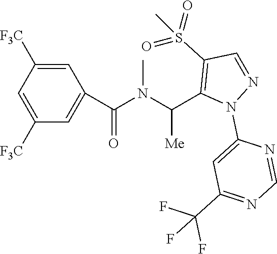 [SMILES: CC(c1c(S(C)(=O)=O)cnn1-c1cc(C(F)(F)F)ncn1)N(C)C(=O)c1cc(C(F)(F)F)cc(C(F)(F)F)c1] | N-methyl-N-(1-(4- (methylsulfonyl)-1-(6- (trifluoromethyl)pyrimidin- 4-yl)-1H-pyrazol-5- yl)ethyl)-3,5- bis(trifluoromethyl) benzamide² |

¹Cas Name
²Chemical Names automatically generated with ChemDraw Professional, Version 17.0.

Embodiment Y1. A composition comprising a compound of Formula 1 or any one of the preceding embodiments and at least one additional component selected from the group consisting of surfactants, solid diluents and liquid diluents, said composition optionally further comprising at least one additional biologically active compound or agent. Embodiment Y2. The composition of embodiment Y1 wherein the at least one additional biologically active compound or agent is selected from the group consisting of abamectin, acephate, acequinocyl, acetamiprid, acrinathrin, afidopyropen, amidoflumet, amitraz, avermectin, azadirachtin, azinphos-methyl, bendiocarb, benfuracarb, bensultap, bifenthrin, bifenazate, bistrifluron, borate, bromantraniliprole, buprofezin, carbaryl, carbofuran, cartap, chlorantraniliprole, chlorfenapyr, chlorfluazuron, chloroprallethrin, chlorpyrifos, chlorpyrifos-methyl, chromafenozide, clofentezine, clothianidin, cyantraniliprole, cyclaniliprole, cyclobutrifluram, cycloprothrin, cycloxaprid, cyetpyrafen, cyflumetofen, cyfluthrin, beta-cyfluthrin, cyhalodiamide, cyhalothrin, gamma-cyhalothrin, lambda-cyhalothrin, cypermethrin, alpha-cypermethrin, zeta-cypermethrin, cyproflanilide, cyromazine, deltamethrin, diafenthiuron, diazinon, dichlorantraniliprole, dieldrin, diflovidazin, diflubenzuron, dimefluthrin, dimehypo, dimethoate, dinotefuran, diofenolan, emamectin, endosulfan, esfenvalerate, ethiprole, etofenprox, etoxazole, fenazaquin, fenbutatin oxide, fenitrothion, fenmezoditiaz, fenothiocarb, fenoxycarb, fenpyroximate, fenpropathrin, fenvalerate, fipronil, flometoquin, flonicamid, fluacrypyrim, fluazaindolizine, flubendiamide, fluchlordiniliprole, flucythrinate, flufenerim, flufenoxuron, flufenoxystrobin, fluensulfone, fluhexafon, flupentiofenox, fluopyram, flupyrimin, flupyradifurone, fluvalinate, tau-fluvalinate, fluxametamide, fonofos, formetanate, fosthiazate, halofenozide, heptafluthrin, hexaflumuron, hexythiazox, hydramethylnon, hydroprene, imidacloprid, indazapyroxamet (N-(1-methylcyclopropyl)-2-(3-pyridinyl)-2H-indazole-4-carboxamide), indoxacarb, insecticidal soaps, isofenphos, isocycloseram, kappa-tefluthrin, kinoprene, lufenuron, malathion, meperfluthrin, metaflumizone, metaldehyde, methamidophos, methidathion, methiocarb, methomyl, methoprene, methoxychlor, methoxyfenozide, metofluthrin, monocrotophos, monofluorothrin, nicofluprole, nicotine, N-[1,1-dimethyl-2-(methylthio)ethyl]-7-fluoro-2-(3-pyridinyl)-2H-indazole-4-carboxamide, N-[1,1-dimethyl-2-(methylsulfinyl)ethyl]-7-fluoro-2-(3-pyridinyl)-2H-indazole-4-carboxamide, N-[1,1-dimethyl-2-(methylsulfonyl)ethyl]-7-fluoro-2-(3-pyridinyl)-2H-indazole-4-carboxamide, N-(1-methylcyclopropyl)-2-(3-pyridinyl)-2H-indazole-4-carboxamide, N-[1-(difluoromethyl)cyclopropyl]-2-(3-pyridinyl)-2H-indazole-4-carboxamide, nitenpyram, nithiazine, novaluron, noviflumuron, oxamyl, oxazosulfyl, parathion, parathion-methyl, permethrin, phorate, phosalone, phosmet, phosphamidon, pirimicarb, profenofos, profluthrin, propargite, protrifenbute, pyflubumide, pymetrozine, pyrafluprole, pyrethrins (pyrethrum), pyridaben, pyridalyl, pyrifluquinazon, pyrimidifen, pyriminostrobin, pyriprole, pyriproxyfen, rotenone, ryanodine, silafluofen, spidoxamat, spinetoram, spinosad, spirobudifen, spirodiclofen, spiromesifen, spirotetramat, sulprofos, sulfoxaflor, tebufenozide, tebufenpyrad, teflubenzuron, tefluthrin, kappa-tefluthrin, terbufos, tetrachlorantraniliprole, tetrachlorvinphos, tetramethrin, tetramethylfluthrin, tetraniliprole, thiacloprid, thiamethoxam, thiodicarb, thiosultap, thiosultap-sodium, tiorantraniliprole, tioxazafen, tolfenpyrad, tralomethrin, triazamate, trichlorfon, trifluenfuronate, triflumezopyrim, triflumuron, tyclopyrazoflor, Bacillus sphaericus, Bacillus thuringiensis delta-endotoxins, entomopathogenic bacteria, entomopathogenic viruses and entomopathogenic fungi, botanical essence including synthetic extracts and unrefined oils, RNA interference mediated target suppressors.
Embodiment Y3. The composition of embodiment Y2 wherein the at least one additional biologically active compound or agent is selected from the group consisting of abamectin, acetamiprid, acrinathrin, afidopyropen, amitraz, avermectin, azadirachtin, benfuracarb, bensultap, bifenthrin, buprofezin, carbaryl, cartap, chlorantraniliprole, chlorfenapyr, chlorpyrifos, clothianidin, cyantraniliprole, cyclaniliprole, cycloprothrin, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenitrothion, fenothiocarb, fenoxycarb, fenvalerate, fipronil, flometoquin, flonicamid, flubendiamide, flufenoxuron, flufenoxystrobin, fluensulfone, flupiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiocarb, methomyl, methoprene, methoxyfenozide, metofluthrin, monofluorothrin, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumezopyrim, triflumuron, Bacillus thuringiensis delta-endotoxins, all strains of Bacillus thuringiensis and all strains of nuclear polyhedrosis viruses.
Embodiment Y4. The composition of any one of embodiments Y1-Y3 further comprising a liquid fertilizer.
Embodiment Y5. The composition of Embodiment Y4 wherein the liquid fertilizer is aqueous-based.
Embodiment Y6. A soil drench formulation comprising the composition of any one of embodiments Y1-Y3.
Embodiment Y7. A spray composition comprising the composition of any one of embodiments Y1-Y3 and a propellant.
Embodiment Y8. A bait composition, comprising the composition of any one of embodiments Y1-Y3, one or more food materials, optionally an attractant, and optionally a humectant.
Embodiment Y9. A trap device for controlling and combating an invertebrate pest, comprising: the bait composition of Embodiment Y8 and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.
Embodiment Y10. A composition comprising the composition of any of Embodiments Y1-Y3 wherein the composition is a solid composition selected from dusts, powders, granules, pellets, prills, pastilles, tablets, and filled films.
Embodiment Y11. The composition of Embodiment Y10 wherein the composition is water-dispersible or water-soluble.
Embodiment Y12. A liquid or dry formulation comprising the composition of any one of Embodiments Y1-Y3 for use in a drip irrigation system, furrow during planting, handheld sprayer, backpack sprayer, boom sprayer, ground sprayer, aerial application, unmanned aeriavehicle, or a seed treatment.
Embodiment Y13. The liquid or dry formulation of Embodiment Y12 wherein said formulation is sprayed at an ultra-low volume.

Of note is that compounds of this invention are characterized by favorable metabolic and/or soil residual patterns and exhibit activity controlling and combating a spectrum of agronomic and nonagronomic invertebrate pests.

Of particular note, for reasons of invertebrate pest control spectrum and economic importance, protection of agronomic crops from damage or injury caused by invertebrate pests by controlling and combating invertebrate pests are embodiments of the invention. Compounds of this invention because of their favorable translocation properties or systemicity in plants also protect foliar or other plant parts which are not directly contacted with a compound of Formula 1 or a composition comprising the compound.

Bioaccumulation of pesticides in non-target organisms is an important safety consideration and it is often desirable to limit the systemic exposure and/or accumulation of pesticides and/or their metabolites in non-target organisms. For example, if a compound is to be applied as an insecticide to a crop plant, it is desirable that the compound does not accumulate in the plasma or fat of a vertebrate animal.

Compounds of Formula 1 may show favorable pharmacokinetic properties in vertebrate animals. In particular, compounds of Formula 1 have been found to have rapid clearance from vertebrate animal plasma/blood and a low distribution into vertebrate animal fat, thus reducing the possibility of unwanted bioaccumulation. Of note is the fluorine atom at the 4-position of the phenyl ring attached to the 5-position of the isoxazoline ring.

The pharmacokinetic properties of compounds of Formula 1 can be measured using a wide variety of assay protocols known in the science of pharmacology. In one illustrative method involving a single oral dose, three male and three female rats each receive a single dose of a test substance via oral gavage. Blood is collected via tail vein at 0.25, 0.5, 1, 2, 4, 8, 12 and 24 h, and then every 24 h thereafter until sacrifice. To process the samples to plasma, blood is collected in tubes containing ethylenediaminetetracetic acid (EDTA) and centrifuged at approximately 3000 rpm to separate plasma from red blood cells. Alternatively, blood is collected using microcapillary tubes and dispensed into tubes containing HPLC water (1:1, v/v). Fat is also collected, homogenized and extracted to determine the concentration of the compound of Formula 1 at sacrifice. The plasma or blood and fat are analyzed for the compound of Formula 1 and/or metabolites, for example, by high-performance liquid chromatography (HPLC) with tandem mass spectrometry detection (LC/MS/MS) to determine the concentration of the test substance. The plasma or blood pharmacokinetic data is analyzed using nonlinear modeling software (e.g., Phoenix® WinNonlin®, Pharsight-A Certara™ Company, St. Louis, MO, U.S.A.) to determine the plasma/blood half-life of the compound of Formula 1, the time after administration when the maximum plasma/blood concentration is reached (Tₘₐₓ), the maximum plasma/blood concentration (Cₘₐₓ), and the area under the plasma/blood concentration curve (AUC). As analysis of fat requires rat sacrifice, fat data is obtained at single time points (i.e. the time of rat sacrifice). The fat:plasma or fat:blood ratio of the compound of Formula 1 is then determined.

Also noteworthy as embodiments of the present invention are compositions comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent.

Further noteworthy as embodiments of the present invention are compositions for controlling and combating an invertebrate pest comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent. Embodiments of the invention further include methods for controlling and combating an invertebrate pest comprising contacting the invertebrate pest or its environment with a biologically effective amount of a compound of any of the preceding Embodiments (e.g., as a composition described herein).

Embodiments of the invention also include a composition comprising a compound of any of the preceding Embodiments, in the form of a soil drench liquid formulation. Embodiments of the invention further include methods for controlling and combating an invertebrate pest comprising contacting the soil with a liquid composition as a soil drench comprising a biologically effective amount of a compound of any of the preceding Embodiments.

Embodiments of the invention also include a spray composition for controlling and combating an invertebrate pest comprising a biologically effective amount of a compound of any of the preceding Embodiments and a propellant. Embodiments of the invention further include a bait composition for controlling and combating an invertebrate pest comprising a biologically effective amount of a compound of any of the preceding Embodiments, one or more food materials, optionally an attractant, and optionally a humectant. Embodiments of the invention also include a device for controlling and combating an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

Embodiments of the invention also include methods for protecting a seed from an invertebrate pest comprising contacting the seed with a biologically effective amount of a compound of any of the preceding Embodiments.

Embodiments of the invention also include methods for controlling and combating an invertebrate pest comprising contacting the invertebrate pest or its environment with a biologically effective amount of a compound of Formula 1 (e.g., as a composition described herein), provided that the methods are not methods of medical treatment of a human body by therapy.

This invention also relates to such methods wherein the invertebrate pest or its environment is contacted with a composition comprising a biologically effective amount of a compound of Formula 1, and at least one additional component selected from the group consisting of surfactants, solid diluents and liquid diluents, said composition optionally further comprising a biologically effective amount of at least one additional biologically active compound or agent, provided that the methods are not methods of medical treatment of a human body by therapy.

The compounds of Formula 1 can be prepared by one or more of the following methods and variations as described in Schemes 1-13. The definitions of substituents in the compounds of Formulae 1-14 below are as defined above in the Summary of the Invention unless otherwise noted. The following abbreviations may be used: DMF is N,N-dimethylformamide, and DBU is 1,8-diazabicyclo[5.4.0]undec-7-ene.

Compounds of Formula 1 in which X is S can be prepared by treating compounds of Formula 1 in which X is O with such sulfurizing agents as phosphorus pentasulfide or Lawesson's reagent (2,4-di(p-methoxyphenyl)-1,3-dithiadiphosphetane disulfide) by conditions and procedures well-known to one skilled in the art of organic synthesis.
As shown in Scheme 1, compounds of Formula 1 in which X is O can be prepared by reaction of an amine intermediate of Formula 2, with a carboxylic acid or acid derivative of Formula 3 using amide coupling agents and conditions well-known to one skilled in the art. For example, 3 in which Y is chloride or fluoride is combined with 2, optionally in the presence of a base such as sodium bicarbonate, potassium hydroxide, or an amine such as triethylamine, diisopropylethylamine, or pyridine, generally in a solvent or solvent mixture such as dichloromethane, ethyl acetate, tetrahydrofuran, or toluene, optionally in the presence of water, at temperatures from below ambient to the boiling point of the reaction mixture. Intermediates 3 in which Y is hydroxyl can react in the presence of an activating agent such as dicyclohexylcarbodiimide, 1,1′-carbonyldiimidazole, or (1-[bis(dimethylamino)methylene]-1H-1,2,3-triazolo[4,5-b]pyridinium 3-oxide hexafluorophosphate (HATU) under conditions well-known in the art. Intermediates 3 in which Y is alkoxy, typically methoxy or ethoxy, can react using promotion by trialkyl aluminum reagents such as trimethylaluminum, in a solvent such as toluene or dichloromethane, to prepare compounds of Formula 1.
Compounds of Formula 3 are commercially available or can be prepared by procedures well-known to one skilled in the art.

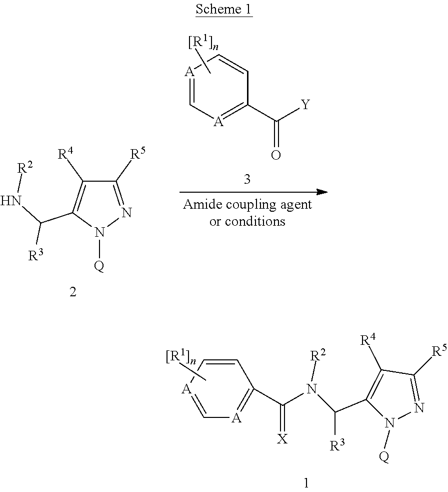

As shown in Scheme 2, intermediates 2 can be prepared by reductive amination of ketones of Formula 4. Such reactions are well-known in the art, and consist of the combination of ketones 4 with amines R²NH₂ and a reducing agent, such as sodium cyanoborohydride or sodium triacetoxyborohydride, in a solvent such as methanol, ethanol, or water, often with addition of an acid promoter such as acetic acid. Conversion of 4 to 2 may be improved by addition of an optional dehydrating agent such as titanium ethoxide or titanium isopropoxide, thus forming an intermediate imine that is reduced by subsequent addition of a reducing agent such as sodium borohydride, or by exposure to hydrogen in the presence of a catalyst such as palladium on carbon support, under conditions well-known in the art. Use of titanium tetraalkoxide and a reducing agent can be helpful to prepare compounds of Formula 1 in which R² is S(O)ₚR⁷ or SO₂NR⁷R⁸, and triethylsilane combined with catalytic bismuth trichloride, as described in Tetrahedron Letters 2014, 55(10), 1829-1834, or triethylsilane and catalytic dirhenium heptoxide, as described in Chemical Communications 2012, 48(66), 8276-8278, can be helpful to prepare compounds of Formula 1 in which R² is C(O)OR⁷.

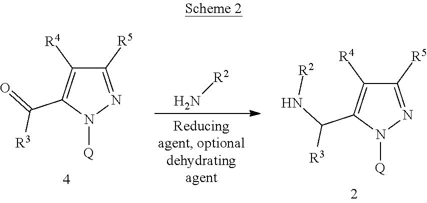

A variation of the approach of Scheme 2 to afford compounds of Formula 2 in which R² is hydrogen is shown in Scheme 3. Combination of ketones 4 with 2-methyl-2-propanesulfinamide, which is available as the (R) or (S) enantiomer, under dehydrating conditions with a titanium tetraalkoxide such as titanium tetraethoxide, followed by reduction with such reagents as sodium borohydride, and subsequent removal of the sulfinyl group by exposure to an acid such as hydrochloric acid, typically in aqueous methanol, 1,4-dioxane or tetrahydrofuran, provides amines 2 in predominantly one enantiomer. Single enantiomers of compounds of Formula 1 may provide advantages in activity, safety, and/or physical properties or other characteristics.

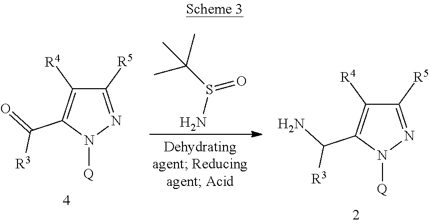

As shown in Scheme 4, compounds of Formula 4 can be prepared by metalation of compounds of Formula 5 and contact with a carbonyl-containing partner. Such metalations can be conducted by reaction with strong lithium bases such a lithium diispropylamide or lithium tetramethylpiperidide, or magnesium bases such as isopropyl magnesium chloride, optionally in a complex with lithium chloride and/or other metal salts to promote the metalation reaction. Suitable solvents are ethers such as diethyl ether or tetrahydrofuran, optionally in the presence of a cosolvent such as N,N′-dimethylpropyleneurea (DMPU). The reaction can be conducted at temperatures from below −70 degrees to ambient temperature or slightly above. Suitable carbonyl compounds are, for example, a “Weinreb amide” R₃C(O)N(Me)OMe, which affords the ketone 4 directly upon hydrolytic work-up.

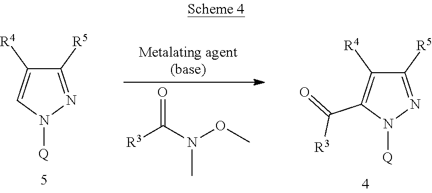

Optionally, the carbonyl compound combined with metalated pyrazoles 5 can be an aldehyde R³CHO, as shown in Scheme 5, affording an intermediate alcohol 6. These alcohols can then be oxidized to afford ketones 4, such as by contact with chromium trioxide, manganese dioxide, or 1,1,1-triacetoxy-1,1-dihydro-1,2-benziodoxol-3(1H)-one (Dess-Martin periodinane, Martin's reagent), in such solvents as dichloromethane, acetonitrile, or acetone, or under a number of other reaction conditions and methods well-known to one skilled in the art.

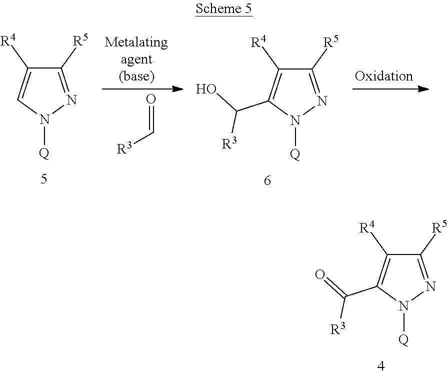

Alternatively, as shown in Scheme 6, alcohols of Formula 6 can be used to prepare amines of Formula 2 in which R² is hydrogen by conversion to an azide 7, such as by reaction of 6 with diphenylphosphoryl azide under Mitsunobu conditions by the addition of an azodicarboxylate ester, such as azodicarboxylic acid diethyl ester (DEAD), and triphenylphosphine, typically in tetrahydrofuran solvent at temperatures slightly below ambient to ambient. Azides 7 can then be reduced to amines 2 using such reagents as triphenyl phosphine in aqueous tetrahydrofuran, stannous chloride in methanol or ethanol, or via hydrogenation over palladium catalyst in solvents such as methanol, ethanol, or ethyl acetate.

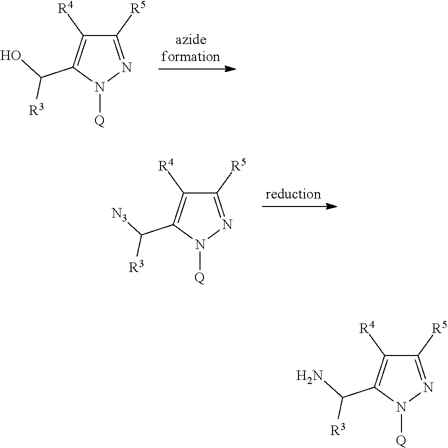

As shown in Scheme 7, amines of Formula 2 in which R² is hydrogen can be used to prepare other amines of Formula 2 with R² other than hydrogen, C(O)R⁷, C(O)OR⁷, NR⁷R⁸, OR⁷, S(O)ₚR⁷ or SO₂NR⁷R⁸, by use of reductive amination conditions similar to those of Scheme 2, and as well-known in the art.

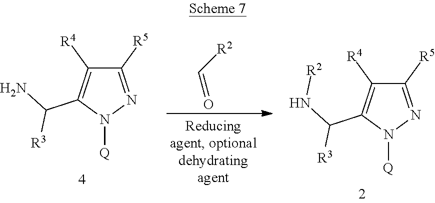

As shown in Scheme 8, pyrazoles of Formula 5 can be prepared by reaction of pyrazoles of Formula 8 with a group Q-L, in which L is a halogen, alkyl- or haloalkylsulfonate, nitro group, or alkysulfonyl group, optionally in the presence of a base promoter, and optionally with a metal catalyst. Possible optional bases include triethylamine, 1,8-diazabicyclo[5.4.0]undec-7-ene (DBU), potassium carbonate, cesium carbonate, sodium hydride, or potassium tert-butoxide, and optional metal catalysts are typically copper or palladium, generally with an added ligand such as N,N′-dimethylethylenediamine, 1,2-cyclohexanediamine, or 2,2′-bipyridine for copper-promoted reactions, or arylphosphine, alkylphosphine or mixed alkyl/arylphosphine ligands for palladium-promoted reactions. Typical solvents for these reactions include water, methanol, tert-butanol, tetrahydrofuran, dimethylsulfoxide, dimethylformamide, 1,4-dioxane, and toluene. Such reactions are well-known to one skilled in the art.

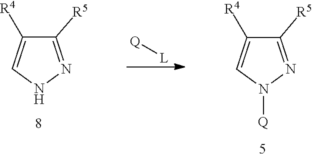

Pyrazoles of Formula 8 and groups Q-L are commercially available, or are readily prepared by methods known to one skilled in the art.
Alternatively, as shown in Scheme 9, compounds of Formula 1 can be prepared by combination of pyrazoles of Formula 9 with a compound of formula Q-L, under conditions such as those described for Scheme 8, above.

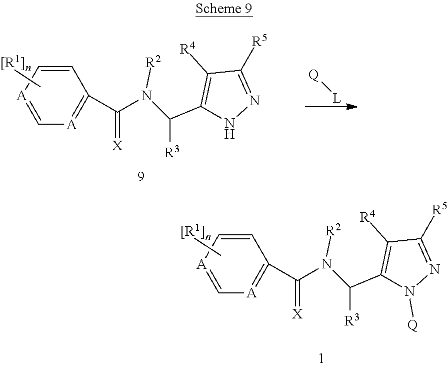

Compounds of Formula 9 can be prepared by methods similar to those of Schemes 1 through 8 either directly, or in some cases advantageously by use of a protecting group on the pyrazole as shown in Scheme 10 for pyrazoles of Formula 10. Some examples of suitable protecting groups are benzyl or para-methoxybenzyl. These protecting groups are removed by methods well-known in the art, such as by hydrogenation to remove benzyl groups, or treatment with mild oxidants such as cerium ammonium nitrate or acids such as trifluoroacetic acid to remove para-methoxybenzyl or related groups.

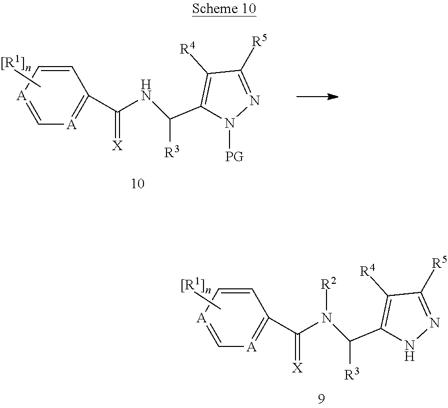

As shown in Scheme 11, compounds of Formula 1 in which R² is hydrogen can be converted to other compounds of Formula 1 by reaction of a group R²-L, with L being an appropriate leaving group such as halogen, methanesulfonate, or para-tolylsulfonate. These reactions are generally carried out in the presence of a base promoter such as sodium hydride, potassium tert-butoxide, or potassium bis(trimethylsilyl)amide, in such solvents as tetrahydrofuran, dimethylsulfoxide, or dimethylformamide.

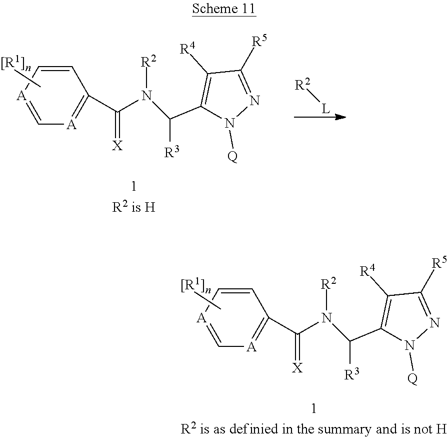

Optionally, Pyrazoles of Formula 4 can be prepared as shown in Schemes 12 and 13. As shown in Scheme 12, hydrazones of Formula 12 can be prepared by the reaction of glyoxal and substituted hydrazine in water or acetic acid as solvent as described by Blake, James F.; et al. World Intellectual Property Organization, WO 2007103308. Typically, these reactions are conducted at room temperature to 125° C. and water can be removed by forming an azeotrope or distillation to give the hydrazones of Formula 12. Hydrazines of Formula 14 are commercially available or are readily prepared by methods known to one skilled in the art. Hydrazones of Formula 12 can be cyclized to pyrazoles of Formula 4 through condensation under basic conditions with compounds of Formula 11, where X is a halogen, as described by Taniguchi, Takahiko; et al. World Intellectual Property Organization, WO 2012020780. Typically, these reactions are conducted in solvents such as 1,4-dioxane at temperatures ranging from room temperature to 150° C. The unsubstituted pyrazole can be further derivatirized to a variety of compounds where R⁴ or R⁵ are such substituents as bromine or cyano under a number of other reaction conditions and methods well-known to one skilled in the art.

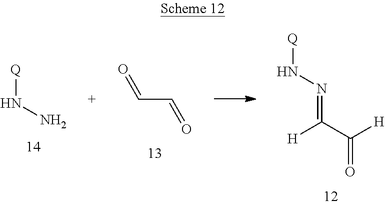

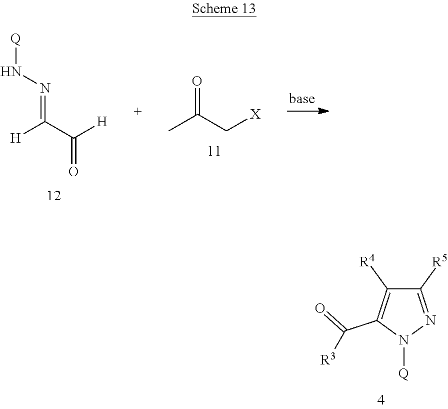

One skilled in the art of organic synthesis will recognize that many substituents such as R¹, R², R⁴, R⁵ and substituents on Q can be interconverted by known chemistry of functional group conversions by reaction with suitable reagents, and are thus not described in detail here due to their familiarity. However, as general illustration, some such conversions might be replacement of hydrogen atoms by halogens using electrophilic halogenating reagents such as N-chlorosuccinimide, 1,3-dibromo-5,5-dimethylhydantoin, and N-fluorobenzenesulfonimide under neutral conditions, or after use of a base such as lithium diisopropylamide to remove a hydrogen atom from the substrate. Halogens are extremely useful functional groups for the introduction of many other substituent groups, such as by radical reactions or nucleophilic displacements, or for introduction of cyano-, carbonyl-, amine-, or sulfur-based groups, generally with catalysis by palladium, nickel, or copper. Other groups can be interconverted by the following: reductions with various well-known reagents such as lithium tetrahydroaluminate or by hydrogenation over palladium catalyst; oxidation with well-known reagents such as manganese dioxide, chromium trioxide or Dess-Martin periodinane; dealkylation of alkoxy groups by contact with boron tribromide or aluminum trichloride; alkylations of oxygen-, nitrogen- and sulfur-based groups with various reagents; nitration of aromatic and heteroaromatic rings; acylation of amines; sulfonylation of amines and hydroxy groups; or preparation of amides from carboxylic acids or esters, etc. A number of these interconversions are illustrated by steps in the Examples below.
Examples of intermediates useful in the preparation of compounds of this invention are shown in Tables I-1 through I-53. The following abbreviations are used in the Tables which follow: Me means methyl, Et means ethyl, Ph means phenyl, C(O) means carbonyl and CHO means formyl.

TABLE I-1

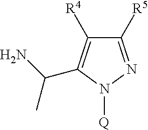

Q is Q-1

| R⁴ | R⁵ |
|---|---|
| H | H |
| H | NO₂ |
| H | NH₂ |
| H | NHMe |
| H | OMe |
| H | SMe |
| Br | H |
| Br | NO₂ |
| Br | NH₂ |
| Br | NHMe |
| Br | OMe |
| Br | SMe |
| I | H |
| I | NO₂ |
| I | NH₂ |
| I | NHMe |
| I | OMe |
| I | SMe |
| CN | H |
| CN | NO₂ |
| CN | NH₂ |
| CN | NHMe |
| CN | OMe |
| CN | SMe |
| SO₂Me | H |
| CO₂H | H |
| CO₂Me | H |
| SO₂Me | NH₂ |
| CO₂H | NH₂ |
| CO₂Me | NH₂ |
| SO₂Me | NHMe |
| CO₂H | NHMe |
| CO₂Me | NHMe |
| SO₂Me | OMe |
| CO₂H | OMe |
| CO₂Me | OMe |
| SO₂Me | SMe |
| CO₂H | SMe |
| CO₂Me | SMe |

Table I-2 is identical to Table I-1, except that Q is Q-2.
Table I-3 is identical to Table I-1, except that Q is Q-3.
Table I-4 is identical to Table I-1, except that Q is Q-4.
Table I-5 is identical to Table I-1, except that Q is Q-5.
Table I-6 is identical to Table I-1, except that Q is Q-6.
Table I-7 is identical to Table I-1, except that Q is Q-7.
Table I-8 is identical to Table I-1, except that Q is Q-8.
Table I-9 is identical to Table I-1, except that Q is Q-9.
Table I-10 is identical to Table I-1, except that Q is Q-10.
Table I-11 is identical to Table I-1, except that Q is Q-11.
Table I-12 is identical to Table I-1, except that Q is Q-12.
Table I-13 is identical to Table I-1, except that Q is Q-13.
Table I-14 is identical to Table I-1, except that Q is Q-14.
Table I-15 is identical to Table I-1, except that Q is Q-15.
Table I-16 is identical to Table I-1, except that Q is Q-16.
Table I-17 is identical to Table I-1, except that Q is Q-17.
Table I-18 is identical to Table I-1, except that Q is Q-18.
Table I-19 is identical to Table I-1, except that Q is Q-19.
Table I-20 is identical to Table I-1, except that Q is Q-20.
Table I-21 is identical to Table I-1, except that Q is Q-21.
Table I-22 is identical to Table I-1, except that Q is Q-22.
Table I-23 is identical to Table I-1, except that Q is Q-23.
Table I-24 is identical to Table I-1, except that Q is Q-24.
Table I-25 is identical to Table I-1, except that Q is Q-25.
Table I-26 is identical to Table I-1, except that Q is Q-26.

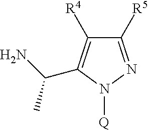

Tables I-27 is identical to Table I-1 except that the structure under the heading “Table I-1” is replaced by the structure shown above.
Tables I-28 through I-53 are identical to Tables I-2 through I-26 except that the structure under the heading “Table I-1” is replaced with the structure shown under “Table I-27”.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after introduction of the reagents depicted in the individual schemes, additional routine synthetic steps not described in detail may be needed to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Synthesis Examples are, therefore, to be construed as merely illustrative, and not limiting of the disclosure in any way whatsoever. Steps in the following Synthesis Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, “dt” means doublet of triplets, “br s” means broad singlet. DMF means N,N-dimethylformamide and DMSO means dimethylsulfoxide. Compound numbers refer to Index Table A.

### Synthesis Example 1

### Preparation of N-[1-[2-(5-bromo-2-pyridyl)-5-nitro-pyrazol-3-yl]ethyl]-N-methyl-3,5-bis(trifluoromethyl)benzamide (Compound 9)

### Step A: Preparation of 5-bromo-2-(3-nitropyrazol-1-yl)pyridine

In a microwave vial, 3-nitro-1H-pyrazole (2.00 g, 17.7 mmol, 1 eq) and 2,5-dibromopyridine (4.10 g, 17.7 mmol, 1 eq) were combined in N,N-dimethylformamide (15 mL), and cesium carbonate (5.7 g, 17.7 mmol, 1 eq) was added. The reaction mixture was irradiated under microwave at 140° C. for 2 h and monitored by thin-layer chromatography. Upon consumption of the starting material, the reaction mixture was poured into cold water (100 mL), and the solid obtained was collected on a frit and washed with water. Drying under reduced pressure afforded 5-bromo-2-(3-nitropyrazol-1-yl)pyridine (3.5 g, 74%) as an off-white solid melting at 215-218° C.

¹H NMR (DMSO-d₆) δ 8.84 (d, 1H), 8.73 (m, 1H), 8.35 (m, 1H), 7.96 (m, 1H), 7.35 (d, 1H). LCMS: m/z: 269/271 [M+H]+

### Step B: Preparation of 1-[2-(5-bromo-2-pyridyl)-5-nitro-pyrazol-3-yl]ethanone

A mixture of 5-bromo-2-(3-nitropyrazol-1-yl)pyridine prepared as in Step 1 (5.5 g, 20.522 mmol, 1 eq) in tetrahydrofuran (350 mL) was cooled to −30° C., lithium diisopropylamide (2M solution in tetrahydrofuran, 15.4 mL, 30.8 mmol, 1.5 eq) was added dropwise, and the mixture was stirred at −30° C. After 40 min. N-methoxy-N-methyl-acetamide (3.1 g, 30.8 mmol, 1.5 eq) was added, and the reaction mixture was allowed to warm slowly to ambient temperature over 2h. Saturated aqueous ammonium chloride solution was added (100 mL), and this mixture was extracted twice with 200 mL portions of ethyl acetate. The combined organic phases were washed twice with 100 mL portions of saturated aqueous sodium chloride solution, dried over sodium sulfate, and concentrated under reduced pressure to obtain a residue. This residue was subjected to combi-flash chromatography, eluting with a gradient of a 0-50% gradient of ethyl acetate in petroleum ether to obtain 1-[2-(5-bromo-2-pyridyl)-5-nitro-pyrazol-3-yl]ethanone (2.3 g, 36%) as a yellow solid melting at 117-120° C. 1H NMR (CDCl₃) δ 8.52 (m, 1H), 8.07 (m, 1H), 7.70 (m, 1H), 7.09 (m, 1H), 2.57 (s, 3H). LCMS: m/z: 311/313 [M+H]+

### Step C: Preparation of 1-[2-(5-bromo-2-pyridyl)-5-nitro-pyrazol-3-yl]-N-methyl-ethanamine

To a mixture of 1-[2-(5-bromo-2-pyridyl)-5-nitro-pyrazol-3-yl]ethanone prepared as in Step 2 (2.8 g, 9.0 mmol, 1 eq) and titanium tetraethoxide (2.6 g, 11.7 mmol, 1.3 eq) in methanol (77 mL) was added methylamine (30% solution in ethanol, 2.8 mL, 27 mmol, 3 eq). The reaction mixture was stirred at ambient temperature for 16 h, then heated at 60° C. for 2 h. The reaction mixture was cooled to 0° C. and sodium borohydride (0.341 g, 9.03 mmol, 1.0 eq) was added. The reaction mixture was stirred at ambient temperature for 2h, and then it was poured into ice-cold water (200 mL) and extracted twice with 150 mL portions of ethyl acetate. The combined organic phases were washed twice with 100 mL portions of saturated aqueous sodium chloride solution, dried over sodium sulfate, and concentrated under reduced pressure to obtain crude 1-[2-(5-bromo-2-pyridyl)-5-nitro-pyrazol-3-yl]-N-methyl-ethanamine (1.8 g, 62%) as an impure, brown semi-solid melting at 125-128° C. LCMS: m/z: 326/328 [M+H]+ as one component. Used directly in reaction of Step 4 below.

### Step D: Preparation of N-[1-[2-(5-bromo-2-pyridyl)-5-nitro-pyrazol-3-yl]ethyl]-N-methyl-3,5-bis(trifluoromethyl)benzamide

To a mixture of 1-[2-(5-bromo-2-pyridyl)-5-nitro-pyrazol-3-yl]-N-methyl-ethanamine prepared as in Step 3 (1.8 g, 5.5 mmol, 1 eq) and 3,5-bis(trifluoromethyl)benzoic acid (1.70 g, 6.65 mmol, 1.2 eq) in N,N-dimethylformamide (18 mL) was added 1-[bis(dimethylamino)methylene]-1H-1,2,3-triazolo[4,5-b]pyridinium 3-oxide hexafluorophosphate (HATU, 3.15 g, 8.31 mmol, 1.5 eq), and diisopropylethylamine (2.0 mL, 11 mmol, 2 eq). The reaction mixture was stirred at ambient temperature for 16h, poured into cold water (80 mL), and extracted twice with 150 mL portions of ethyl acetate. The combined organic phases were washed twice with 100 mL portions of saturated aqueous sodium chloride solution, dried over sodium sulfate, and concentrated under reduced pressure to obtain a crude residue. This residue was purified by combi-flash chromatography, eluting with a gradient of 0-50% ethyl acetate in petroleum ether to obtain N-[1-[2-(5-bromo-2-pyridyl)-5-nitro-pyrazol-3-yl]ethyl]-N-methyl-3,5-bis(trifluoromethyl)benzamide (1.8 g, 58%), a compound of this invention, as an off-white solid melting at 174-177° C. Some amount of the product of Step B co-eluted with this product. 1H NMR (CDCl₃) δ 8.45 (m, 1H), 8.1 (m, 1H), 7.9 (m, 2H), 7.4-7.5 (m, 2H), 7.2 (m, 1H), 6.5 (q, 1H), 2.60 (s, 3H), 1.74 (d, 3H).

LCMS: m/z: 566/568 [M+H]+

### Synthesis Example 2

### Preparation of N-[1-[5-amino-2-(5-bromo-2-pyridyl)pyrazol-3-yl]ethyl]-N-methyl-3,5-bis(trifluoromethyl)benzamide (Compound 11)

To a mixture of N-[1-[2-(5-bromo-2-pyridyl)-5-nitro-pyrazol-3-yl]ethyl]-N-methyl-3,5-bis(trifluoromethyl)benzamide prepared as in Example 1 (1.10 g, 1.95 mmol, 1 eq) in ethanol (50 mL) and water (25 mL), was added ammonium chloride (415 mg, 7.79 mmol, 4 eq) and iron powder (217 mg, 3.89 mmol, 2 eq). The reaction mixture was stirred at reflux for 4 h, then allowed to cool to ambient temperature. The reaction mixture was diluted with ethyl acetate (100 mL), filtered through a pad of Celite filter aid, and the filter cake was washed with ethyl acetate (100 mL). The combined organic layers were washed twice with 100 mL portions of saturated aqueous sodium chloride solution, dried over sodium sulfate, and concentrated under reduced pressure to obtain a crude residue. The crude material was purified by combi-flash chromatography, eluting with a gradient of 0-80% ethyl acetate/petroleum ether to obtain N-[1-[5-amino-2-(5-bromo-2-pyridyl)pyrazol-3-yl]ethyl]-N-methyl-3,5-bis(trifluoromethyl)benzamide (800 mg, 80%), a product of this invention, as a brown solid melting at 139-141° C. ¹H NMR (DMSO-d₆, 90° C.) δ 7.8-8.25 (m, 3H), 7.4-7.85 (m, 3H), 6.30 (q, 1H), 5.97 (s, 1H), 5.29 (s, 2H), 3.32 (s, 3H), 2.50 (d, 3H).

LCMS: m/z: 536/538 [M+H]+

### Synthesis Example 3

### Preparation of N-[1-[5-acetamido-2-(5-bromo-2-pyridyl)pyrazol-3-yl]ethyl]-N-methyl-3,5-bis(trifluoromethyl)benzamide (Compound 15)

In a 20 mL scintillation vial, a mixture of N-{1-[5-amino-2-(5-bromopyridin-2-yl)pyrazol-3-yl]ethyl}-N-methyl-3,5-bis(trifluoromethyl)benzamide prepared as in Example 2 (96.7 mg, 0.18 mmol, 1 eq) in tetrahydrofuran (0.60 mL) was stirred and cooled in an ice bath. Triethylamine (0.077 mL, 0.541 mmol, 3 eq) and acetyl chloride (0.016 mL, 0.22 mmol, 1.2 eq) were added dropwise. The mixture was stirred 16h at ambient temperature, diluted with water, and extracted twice with two portions of about 10 mL of ethyl acetate. The organic phase was washed with saturated aqueous sodium chloride solution, dried over magnesium sulfate, and concentrated to a crude residue under reduced pressure. The crude residue was subjected to automated flash chromatography, eluting with a gradient of 0-50% ethyl acetate/hexanes to isolate N-[1-[5-acetamido-2-(5-bromo-2-pyridyl)pyrazol-3-yl]ethyl]-N-methyl-3,5-bis(trifluoromethyl)benzamide (84 mg), a compound of this invention, as a brown solid.

¹H NMR (CDCl₃) δ 8.35 (m, 1H), 8.20 (m, 1H), 7.85-7.96 (m, 2H), 7.63 (m, 1H), 7.45 (br s, 2H), 7.11 (m, 1H), 6.50 (br m, 1H), 2.53 (s, 3H), 2.20 (s, 3H), 1.67 (d, 3H).

LCMS: m/z: 578/580 [M+H]+

### Synthesis Example 4

### Preparation of N-[1-[5-amino-2-(5-bromo-2-pyridyl)-4-iodo-pyrazol-3-yl]ethyl]-N-methyl-3,5-bis(trifluoromethyl)benzamide (Compound 10)

To a mixture of N-{1-[5-amino-2-(5-bromopyridin-2-yl)pyrazol-3-yl]ethyl}-N-methyl-3,5-bis(trifluoromethyl)benzamide prepared as in Example 2 (800 mg, 1.5 mmol, 1 eq) in N,N-dimethylformamide (8 mL) was added N-iodosuccinimide (415 mg, 1.64 mmol, 1.1 eq). The reaction mixture was stirred at 40° C. for 3 h, then poured into cold water (100 mL). The obtained solid was collected on a frit and washed with water and dried under reduced pressure to obtain N-[1-[5-amino-2-(5-bromo-2-pyridyl)-4-iodo-pyrazol-3-yl]ethyl]-N-methyl-3,5-bis(trifluoromethyl)benzamide, a compound of this invention, (780 mg, 78%) as an off-white solid melting at 159-162° C. ¹H NMR (DMSO-d₆, 91° C.) δ 8.33 (s, 1H), 8.05 (m, 1H), 7.96 (m, 1H), 7.69 (s, 2H), 7.48 (m, 1H), 6.17 (q, 1H), 4.84 (br, 2H), 2.96 (s, 3H), 1.72 (s, 3H).

LCMS: m/z: 662/664 [M+H]+

### Synthesis Example 5

### Preparation of N-[1-[3-amino-4-cyano-1-(5-cyano-2pyridinyl)-1H-pyrazol-5-yl]ethyl]-N-methyl-3,5 bis(trifluoromethyl) benzamide (Compound 14)

In microwave vial a mixture of N-[1-[5-amino-2-(5-bromo-2-pyridyl)-4-iodo-pyrazol-3-yl]ethyl]-N-methyl-3,5-bis(trifluoromethyl)benzamide prepared as in Example 4 (400 mg, 0.605 mmol, 1 eq) and zinc cyanide (283 mg, 2.42 mmol, 4 eq) in N,N-dimethylformamide (4 mL) was degassed with argon for 15 min, then 1,1′-bis(diphenylphosphino)ferrocene (67 mg, 0.12 mmol, 0.2 eq) and tris(dibenzylideneacetone)dipalladium(0) (55 mg, 0.060 mmol, 0.1 eq) were added. The reaction mixture was irradiated under microwave at 120° C. for 1.5h and then poured into ice cold water (20 mL) and extracted with two 50 mL portions of ethyl acetate. The combined organic layers were washed twice with 50 mL portions of saturated aqueous sodium chloride solution, dried over sodium sulfate, and concentrated under reduced pressure to obtain a crude residue. The crude material was purified by combi-flash chromatography, eluting with a gradient of 0-80% ethyl acetate/petroleum ether to obtain N-[1-[5-amino-4-cyano-2-(5-cyano-2-pyridyl)pyrazol-3-yl]ethyl]-N-methyl-3,5-bis(trifluoromethyl)benzamide (220 mg, 71%), a compound of this invention, as an off white solid melting at 191-194° C. IR 2222 cm¹

¹H NMR (DMSO-d₆, 89° C.) δ 8.76 (s, 1H), 8.38 (m, 1H), 8.06 (s, 1H), 7.8-7.9 (m, 3H), 6.35 (q, 1H), 5.84 (s, 2H), 2.96 (s, 3H), 2.48 (d, 3H).

LCMS: m/z: 508 [M+H]+

### Synthesis Examples 6 and 7

### Preparation of N-[1-[4-cyano-1-(5-cyano-2-pyridinyl)-3-(methylamino)-1H-pyrazol-5-yl]ethyl]-N-methyl-3,5-bis(trifluoromethyl)benzamide (Compound 12) and N-[1-[4-cyano-2-(5-cyano-2-pyridyl)-5-(dimethylamino)pyrazol-3-yl]ethyl]-N-methyl-3,5-bis(trifluoromethyl)benzamide (Compound 13)

In microwave vial, to a mixture of N-[1-[5-amino-4-cyano-2-(5-cyano-2-pyridyl)pyrazol-3-yl]ethyl]-N-methyl-3,5-bis(trifluoromethyl)benzamide prepared as in Example 5 (80 mg, 0.16 mmol, 1 eq) and potassium carbonate (43 mg, 0.32 mmol, 2 eq) in N,N-dimethylformamide (2 mL) was added iodomethane (33 mg, 0.23 mmol, 1.5 eq). The reaction mixture was irradiated under microwave at 100° C. for 1h, poured into ice cold water (20 mL), extracted with two 50 mL portions of ethyl acetate. The combined organic layers were washed twice with 50 mL portions of saturated aqueous sodium chloride solution, dried over sodium sulfate, and concentrated under reduced pressure to obtain a crude residue. The crude material was purified by combi-flash chromatography, eluting with a gradient of 0-80% ethyl acetate/petroleum ether to obtain compound N-[1-[4-cyano-2-(5-cyano-2-pyridyl)-5-(methylamino)pyrazol-3-yl]ethyl]-N-methyl-3,5-bis(trifluoromethyl)benzamide (Compound 12), a compound of this invention (20 mg, 24%), as a pale brown solid. (Compound 12).

¹H NMR (CDCl₃) δ 8.6 (br, 1H), 8.07 (br, 2H), 7.82-7.95 (br m, 3H), 6.6 (br, 1H), 4.22 (q, 1H), 3.1 (s, 3H), 3.02 (d, 3H), 1.83 (d, 3H).

LCMS: m/z: 522 [M+H]+

Also isolated from this reaction was N-[1-[4-cyano-2-(5-cyano-2-pyridyl)-5-(dimethylamino)pyrazol-3-yl]ethyl]-N-methyl-3,5-bis(trifluoromethyl)benzamide (Compound 13), a compound of this invention (15 mg, 17%), as an off-white solid (Compound 13).

¹H NMR (CDCl₃) δ 8.62 (br, 1H), 8.07 (br, 2H), 7.85-7.95 (br, 3H), 6.58 (br, 1H), 3.16 (s, 3H), 3.12 (s, 6H), 1.85 (d, 3H). LCMS: m/z: 536 [M+H]+

### Synthesis Example 8

### Preparation of methyl 6-[3-amino-5-[1-[[3,5-bis(trifluoromethyl)benzoyl]-methyl-amino]ethyl]pyrazol-1-yl]pyridine-3-carboxylate (Compound 30)

In a steel bomb a mixture of N-[1-[2-(5-bromo-2-pyridyl)-5-nitro-pyrazol-3-yl]ethyl]-N-methyl-3,5-bis(trifluoromethyl)benzamide prepared as in Example 1 (500 mg, 0.884 mmol, 1 eq) and triethylamine (0.255 mL, 1.76 mmol, 2 eq) in N,N-dimethylformamide (2 mL) and methanol (10 mL) was degassed with argon for 15 min. [1,1′-bis(diphenylphosphino)ferrocene]dichloropalladium(II) (1:1 complex with dichloromethane) (72 mg, 0.088 mmol, 0.1 eq) was added. Carbon monoxide gas was admitted at a pressure of 150 psi, and the mixture was heated at 100° C. for 16 h. The reaction mixture was poured into cold water (80 mL) and extracted twice with 150 mL portions of ethyl acetate. The combined organic phases were washed twice with 100 mL portions of saturated aqueous sodium chloride solution, dried over sodium sulfate, and concentrated under reduced pressure to obtain a residue.

The crude residue was purified by combi-flash chromatography, eluting with a gradient of 0-80% ethyl acetate in petroleum ether to obtain methyl 6-[3-amino-5-[1-[[3,5-bis(trifluoromethyl)benzoyl]-methyl-amino]ethyl]pyrazol-1-yl]pyridine-3-carboxylate (300 mg, 65%), a compound of this invention, as a brown solid melting at 171-174° C.

¹H NMR (DMSO-d₆, 90° C.) δ 8.55 (br, 1H), 8.29 (d, 1H), 8.00 (s, 1H), 7.75 (d, 1H), 7.61 (s, 2H), 6.3 (br, 1H), 6.05 (s, 1H), 5.12 (s, 2H), 3.83 (s, 3H), 2.65 (br, 3H), 1.57 (d, 3H).

LCMS: m/z: 516 [M+H]+

### Synthesis Example 9

### Preparation of methyl 6-[3-amino-5-[1-[[3,5-bis(trifluoromethyl)benzoyl]-methyl-amino]ethyl]-4-iodo-pyrazol-1-yl]pyridine-3-carboxylate (Compound 23)

To a mixture of methyl 6-[3-amino-5-[1-[[3,5-bis(trifluoromethyl)benzoyl]-methyl-amino]ethyl]pyrazol-1-yl]pyridine-3-carboxylate prepared as in Example 8 (1.5 g, 2.9 mmol, 1 eq) in N,N-dimethylformamide (20 mL) was added N-iodosuccinimide (0.72 g, 3.2 mmol, 1.1 eq), and the reaction mixture was stirred at 40° C. for 3 h and then poured into cold water (200 mL). The obtained solid was collected on a frit, washed with water, and dried under reduced pressure to obtain methyl 6-[3-amino-5-[1-[[3,5-bis(trifluoromethyl)benzoyl]-methyl-amino]ethyl]-4-iodo-pyrazol-1-yl]pyridine-3-carboxylate (1.43 g, 72%), a compound of this invention, as a white solid melting at 163-169° C.

¹H NMR (DMSO-d₆, 89° C.) δ 8.71 (s, 1H), 8.30 (m, 1H), 7.94 (s, 1H), 7.67 (m 3H), 6.35 (q, 1H), 4.99 (s, 2H), 3.87 (s, 3H), 2.98 (s, 3H), 1.75 (d, 3H).

LCMS: m/z: 642 [M+H]+

### Synthesis Example 10

### Preparation of methyl 6-[3-amino-5-[1-[[3,5-bis(trifluoromethyl)benzoyl]-methyl-amino]ethyl]-4-cyano-pyrazol-1-yl]pyridine-3-carboxylate (Compound 24)

In microwave vial a mixture of methyl 6-[3-amino-5-[1-[[3,5-bis(trifluoromethyl)benzoyl]-methyl-amino]ethyl]-4-iodo-pyrazol-1-yl]pyridine-3-carboxylate prepared as in Example 9 (1.0 g, 1.56 mmol, 1 eq) and zinc cyanide (0.365 g, 3.12 mmol, 2 eq) in N,N-dimethylformamide(15 mL) was degassed with argon for 15 min, then 1,1′-bis(diphenylphosphino)ferrocene (0.172 g, 0.312 mmol, 0.2 eq) and tris(dibenzylideneacetone)dipalladium(0) (0.142 g, 0.156 mmol, 0.1 eq) were added. The reaction mixture was irradiated under microwave at 120° C. for 1.5h, then poured into ice cold water (80 mL) and extracted twice with 100 mL portions of ethyl acetate. The combined organic phases were washed twice with 50 mL portions of saturated aqueous sodium chloride solution, dried over sodium sulfate, and concentrated under reduced pressure to obtain a residue. The crude residue was purified by combi-flash chromatography, eluting with a gradient of 0-50% ethyl acetate/petroleum ether to obtain compound methyl 6-[3-amino-5-[1-[[3,5-bis(trifluoromethyl)benzoyl]-methyl-amino]ethyl]-4-cyano-pyrazol-1-yl]pyridine-3-carboxylate (700 mg, 82%), a compound of this invention, as an off-white solid melting at 229-233° C.

¹H NMR (DMSO-d₆, 89° C.) δ 8.79 (s, 1H), 8.42 (d, 1H), 8.03 (s, 1H), 7.77-7.85 (m, 3H), 6.38 (q, 1H), 5.74 (s, 2H), 4.02 (s, 3H), 2.92 (s, 3H), 1.73 (d, 3H).

LCMS: m/z: 541 [M+H]+

### Synthesis Example 11

### Preparation of 6-[3-amino-5-[1-[[3,5-bis(trifluoromethyl)benzoyl]-methyl-amino]ethyl]-4-cyano-pyrazol-1-yl]pyridine-3-carboxylic acid (Compound 25)

To a mixture of methyl 6-[3-amino-5-[1-[[3,5-bis(trifluoromethyl)benzoyl]-methyl-amino]ethyl]-4-cyano-pyrazol-1-yl]pyridine-3-carboxylate prepared as in Example 4 (800 mg, 1.48 mmol, 1 eq) in tetrahydrofuran (40 mL) and water (20 mL), lithium hydroxide monohydrate (124 mg, 2.96 mmol, 2 eq) was added. The reaction mixture was stirred at ambient temperature for 16h, poured into cold water (20 mL), and extracted twice with 50 mL portions of diethyl ether. The aqueous layer was acidified with 1N aqueous hydrochloric acid to pH 4, and the resultant precipitate was collected on a frit, washed with water, dried under reduced pressure to obtain 6-[3-amino-5-[1-[[3,5-bis(trifluoromethyl)benzoyl]-methyl-amino]ethyl]-4-cyano-pyrazol-1-yl]pyridine-3-carboxylic acid (650 mg, 83%), a compound of this invention, as an off-white solid melting at 193-196° C.

¹H NMR (DMSO-d₆, 90° C.) δ 8.74 (s, 1H), 8.34 (m, 1H), 8.02 (s, 1H), 7.83 (s, 2H), 7.64 (m, 1H), 6.31 (q, 1H), 5.63 (s, 2H), 2.91 (s, 3H), 1.69 (d, 3H).

LCMS: m/z: 527 [M+H]+

### Synthesis Example 12

### Preparation of N-[1-[3-amino-4-cyano-1-[5-(4-morpholinylcarbonyl)-2-pyridinyl]-1H-pyrazol-5-yl]ethyl]-N-methyl-3,5-bis(trifluoromethyl)benzamide (Compound 26)

To a mixture of 6-[3-amino-5-[1-[[3,5-bis(trifluoromethyl)benzoyl]-methyl-amino]ethyl]-4-cyano-pyrazol-1-yl]pyridine-3-carboxylic acid prepared as in Example 5 (700 mg, 1.33 mmol, 1 eq), morpholine (115 mg, 1.73, 1.3 eq) and 1-[bis(dimethylamino)methylene]-1H-1,2,3-triazolo[4,5-b]pyridinium 3-oxide hexafluorophosphate (HATU, 758 mg, 2.0 mmol, 1.5 eq), in N,N-dimethylformamide (15 mL) was added diisopropylethylamine (0.73 mL, 4.0 mmol, 3 eq). The reaction mixture was stirred at ambient temperature for 16 h and poured into cold water (60 mL). The obtained solid was collected on a frit and washed with water and dried under reduced pressure to obtain compound N-[1-[5-amino-4-cyano-2-[5-(morpholine-4-carbonyl)-2-pyridyl]pyrazol-3-yl]ethyl]-N-methyl-3,5-bis(trifluoromethyl)benzamide (500 mg, 63%), a compound of this invention, as a white solid melting at 211-214° C.

¹H NMR (DMSO-d₆, 89° C.) δ 8.39 (s, 1H), 8.06 (m, 1H), 8.01 (m, 1H), 7.89 (s, 2H), 7.76 (m, 1H), 6.35 (q, 1H), 5.66 (s, 2H), 3.58 (m, 4H), 3.48 (m, 4H), 2.93 (s, 3H), 1.70 (d, 3H).

LCMS: m/z: 596 [M+H]+

### Synthesis Example 13

### Preparation of methyl 6-[3-acetamido-5-[1-[[3,5-bis(trifluoromethyl)benzoyl]-methyl-amino]ethyl]pyrazol-1-yl]pyridine-3-carboxylate (Compound 27)

To a mixture of methyl 6-[3-amino-5-[1-[[3,5-bis(trifluoromethyl)benzoyl]-methyl-amino]ethyl]pyrazol-1-yl]pyridine-3-carboxylate prepared as in Example 8 (300 mg, 0.58 mmol, 1 eq) and triethylamine (0.167 mL, 1.17 mmol, 2 eq), in dichloromethane (10 mL) was added acetyl chloride (0.061 mL, 0.87 mmol, 1.5 eq) with ice-bath cooling. The reaction mixture was stirred at ambient temperature for 16 h, then combined with ice-cold water (20 mL) and extracted with two 50 mL portions of dichloromethane. The combined organic layers were washed twice with 50 mL portions of saturated aqueous sodium chloride solution, dried over sodium sulfate, and concentrated under reduced pressure to obtain a crude residue. The crude material was purified by combi-flash chromatography, eluting with a gradient of 0-70% ethyl acetate/petroleum ether to obtain compound methyl 6-[3-acetamido-5-[1-[[3,5-bis(trifluoromethyl)benzoyl]-methyl-amino]ethyl]pyrazol-1-yl]pyridine-3-carboxylate (150 mg, 46%), a compound of this invention, as a white solid melting at 266-269° C.

¹H NMR (DMSO-d₆, 89° C.) δ 10.44 (s, 1H), 8.64 (br, 1H), 8.41 (d, 1H), 7.99 (m, 1H), 7.82 (d, 1H), 7.57 (s, 2H), 6.99 (s, 1H), 6.35 (br m, 1H), 3.86 (s, 3H), 2.55-2.85 (br, 3H), 2.06 (s, 3H), 1.62 (d, 3H).

LCMS: m/z: 558 [M+H]+

### Synthesis Example 14

### Preparation of 6-[3-acetamido-5-[1-[[3,5-bis(trifluoromethyl)benzoyl]-methyl-amino]ethyl]pyrazol-1-yl]pyridine-3-carboxylic acid (Compound 28)

To a mixture of methyl 6-[3-acetamido-5-[1-[[3,5-bis(trifluoromethyl)benzoyl]-methyl-amino]ethyl]pyrazol-1-yl]pyridine-3-carboxylate prepared as in Example 13 (120 mg, 0.215 mmol, 1 eq) in tetrahydrofuran (10 mL) and water (5 mL), lithium hydroxide monohydrate (27 mg, 0.65 mmol, 3 eq) was added. The reaction mixture was stirred at ambient temperature for 16h, poured into cold water (15 mL), and extracted twice with 20 mL portions of diethyl ether. The aqueous layer was acidified with 1N aqueous hydrochloric acid to pH 4, and the obtained solid was collected on a frit and washed with water and dried under reduced pressure to obtain 6-[3-acetamido-5-[1-[[3,5-bis(trifluoromethyl)benzoyl]-methyl-amino]ethyl]pyrazol-1-yl]pyridine-3-carboxylic acid (100 mg, 86%), a compound of this invention, as an off-white solid melting at 266-269° C.

¹H NMR (DMSO-d₆, 28° C.) δ 13.4 (br, 1H), 10.83 (s, 1H), 8.71 (s, 1H), 8.48 (d, 1H), 8.15 (s, 1H), 7.84 (d, 1H), 7.41 (s, 2H), 7.05 (s, 1H), 6.44 (m, 1H), 2.39 (s, 3H), 2.07 (s, 3H), 1.60 (d, 3H).

LCMS: m/z: 544 [M+H]+

### Synthesis Example 15

### Preparation of N-[1-[5-acetamido-2-[5-(morpholine-4-carbonyl)-2-pyridyl]pyrazol-3-yl]ethyl]-N-methyl-3,5-bis(trifluoromethyl)benzamide (Compound 21)

To a mixture of 6-[3-acetamido-5-[1-[[3,5-bis(trifluoromethyl)benzoyl]-methyl-amino]ethyl]pyrazol-1-yl]pyridine-3-carboxylic acid prepared as in Example 14 (100 mg, 0.183 mmol, 1 eq), morpholine (20 mg, 0.24 mmol, 1.3 eq) and 1-[bis(dimethylamino)methylene]-1H-1,2,3-triazolo[4,5-b]pyridinium 3-oxide hexafluorophosphate (HATU, 104 mg, 0.275 mmol, 1.5 eq) in N,N-dimethylformamide (3 mL) was added diisopropylethylamine (0.10 mL, 0.55 mmol, 3 eq). The reaction mixture was stirred at ambient temperature for 16 h and poured into cold water (20 mL). The obtained solid was collected on a frit, washed with water, dried under reduced pressure to obtain N-[1-[5-acetamido-2-[5-(morpholine-4-carbonyl)-2-pyridyl]pyrazol-3-yl]ethyl]-N-methyl-3,5-bis(trifluoromethyl)benzamide (103 mg, 46%), a compound of this invention, as white solid melting at 276-279° C.

¹H NMR (DMSO-d₆, 90° C.) δ 10.36 (s, 1H), 8.24 (br s, 1H), 8.02 (s, 1H), 8.00 (d, 1H), 7.75 (d, 1H), 7.69 (s, 2H), 6.95 (s, 1H), 6.32 (br, 1H), 3.55 (br, 4H), 3.44 (br, 4H), 2.6-2.8 (br, 3H), 2.06 (s, 3H), 1.60 (d, 3H).

LCMS: m/z: 613 [M+H]+

### Synthesis Example 16

### Preparation of N-[1-[4-cyano-2-(2-pyridyl)pyrazol-3-yl]ethyl]-N-(cyclopropylmethyl)-3,5-bis(trifluoromethyl)benzamide (Compound 7)

### Step A: Preparation of (2E)-2-(2-pyridylhydrazono)acetaldehyde

A solution of 2-pyridylhydrazine (5.0 g, 45.45 mmol, 1.0 eq) in water (5 mL) was added to 40% glyoxal (6.5 mL, 181.8 mmol, 4.0 eq) at ambient temperature with stirring. The reaction mixture was heated to 100° C. for 4h then cooled to ambient temperature. The precipitated solid was collected on a frit and dried under vacuum to obtain a grey solid (2E)-2-(2-pyridylhydrazono)acetaldehyde (5.8 g, 85%). ¹H NMR (DMSO) δ 12.058 (bs, NH), 9.509 (d, 1H), 8.253 (d, 1H), 7.804 (m, 1H), 7.510 (d, 1H), 7.357 (d, 1H), 7.025 (t, 1H).

### Step B: Preparation of 1-[2-(2-pyridyl)pyrazol-3-yl]ethanone

To a mixture of (2E)-2-(2-pyridylhydrazono)acetaldehyde (5.8 g, 38.9 mmol, 1.0 eq) in 1, 4-dioxane (50 mL) was added potassium carbonate (13.4 g, 97.3 mmol, 2.5 eq) and chloroacetone (4.88 mL, 58.3 mmol, 1.5 eq.) at ambient temperature. The reaction mixture was heated at 80° C. for 5h, concentrated under reduced pressure, diluted with water (100 mL), and extracted twice with two 150 mL portions of ethyl acetate. The combined organic layers were washed with saturated aqueous sodium chloride solution (50 mL), dried over anhydrous sodium sulfate and concentrated under reduced pressure. The obtained crude solid was purified by column chromatography on silica gel (40 g) using combi-flash, eluting with 40% ethyl acetate/petroleum ether to afford 1-[2-(2-pyridyl)pyrazol-3-yl]ethanone (5.1 g, 70%) as an off white solid. ¹H NMR (DMSO-d₆) δ 8.46 (d, 1H), 8.05 (d, 1H), 7.84 (s, 1H), 7.68 (d, 1H), 7.48 (d, 1H), 7.14 (d, 1H), 2.49 (s, 3H)

### Step C: Preparation of 1-[4-bromo-2-(2-pyridyl)pyrazol-3-yl]ethanone

To a solution of 1-[2-(2-pyridyl)pyrazol-3-yl]ethanone (2.00 g, 10.6 mmol, 1.0 eq) in dry N,N-dimethylformamide (20 mL) was added N-bromosuccinimide (2.00 g, 11.7 mmol, 1.1 eq), at 0° C. The resulting reaction mixture was stirred at 0° C. to ambient temperature for 16h, quenched with water (50 mL) and extracted with twice with 100 mL portions of ethyl acetate. The combined organic layers were washed with saturated aqueous sodium chloride solution, dried over anhydrous sodium sulfate and concentrated under reduced pressure to obtain a crude residue. The crude material was purified by column chromatography on silica gel (24 g) eluting with 20% ethyl acetate/petroleum ether to obtain 1-[4-bromo-2-(2-pyridyl)pyrazol-3-yl]ethanone (1.8 g, 64%) as an off-white solid. ¹H NMR (DMSO-d₆) δ 8.44 (d, 1H), 8.08 (m, 2H), 7.87 (d, 1H), 7.46 (t, 1H), 2.48 (s, 3H)

### Step D: Preparation of N-[1-[4-bromo-2-(2-pyridyl)pyrazol-3-yl]ethyl]-1-cyclopropyl-methanimine

To a solution of 1-[4-bromo-2-(2-pyridyl)pyrazol-3-yl]ethanone (1.0 g, 3.7 mmol, 1.0 eq) in dry tetrahydrofuran (20 mL) was added titanium tetraethoxide (3.4 g, 15.0 mmol, 4.0 eq) and cyclopropylmethylamine (236 mg, 4.15 mmol, 1.1 eq) at ambient temperature. The resulting reaction mixture was heated at 70° C. for 16h, water (50 mL) and ethyl acetate (200 mL) were added, and the mixture was filtered through a Celite® pad. The organic layer was separated and washed with saturated aqueous sodium chloride solution, dried over anhydrous sodium sulfate, and concentrated under reduced pressure to obtain a crude residue. The crude material was purified by column chromatography on silica gel (24 g) eluting with 50% ethyl acetate/petroleum ether to obtain 1-[4-bromo-2-(2-pyridyl)pyrazol-3-yl]-N-(cyclopropylmethyl)ethanimine (0.6 g, 55%) as a brown solid. ¹H NMR (DMSO-d₆) δ 8.41 (d, 1H), 8.03 (m, 2H), 7.90 (d, 1H), 7.40 (t, 1H), 2.88 (m, 1H), 2.67 (m, 1H), 2.50 (s, 3H), 1.18 (m, 1H), 0.86 (m, 1H), 0.29 (m, 2H), −0.14 (m, 1H)

### Step E: Preparation of 1-[4-bromo-2-(2-pyridyl)pyrazol-3-yl]-N-(cyclopropylmethyl)ethanamine

To a solution of 1-[4-bromo-2-(2-pyridyl)pyrazol-3-yl]-N-(cyclopropylmethyl)ethanimine (100 mg, 0.31 mmol, 1.0 eq) in methanol (5 mL) was added cerium(III) chloride (115 mg, 0.466 mmol, 1.5 eq), at 0° C., with stirring. After 15 min, sodium borohydride (29 mg, 0.77 mmol, 2.5 eq), was added portion-wise at 0° C. and this mixture was stirred for 16 h. The reaction mixture was poured into ice-cold water (50 mL) and ethyl acetate (200 mL), and filtered through a pad of Celite®. The organic layer was separated and washed with saturated aqueous sodium chloride solution, dried over anhydrous sodium sulfate, and concentrated under reduced pressure to obtain crude 1-[4-bromo-2-(2-pyridyl)pyrazol-3-yl]-N-(cyclopropylmethyl)ethanamine (155 mg), which was used directly in the next step without further purification.

### Step F: Preparation of N-[1-[4-bromo-2-(2-pyridyl)pyrazol-3-yl]ethyl]-N-(cyclopropylmethyl)-3,5-bis(trifluoromethyl)benzamide

To a solution of 3,5-bis(trifluoromethyl)benzoic acid (600 mg, 2.3 mmol, 1.0 eq) in dry DMF (140 mL) were added 1-[bis(dimethylamino)methylene]-1H-1,2,3-triazolo[4,5-b]pyridinium 3-oxide hexafluorophosphate (HATU, 1.32 g, 3.4 mmol, 1.5 eq), crude 1-[4-bromo-2-(2-pyridyl)pyrazol-3-yl]-N-(cyclopropylmethyl)ethanamine obtained as in Step E (821 mg, 2.5 mmol, 1.1 eq) and diisopropylethylamine (1 mL, 5.8 mmol, 2.5 eq) at ambient temperature. The resulting reaction mixture was stirred at ambient temperature for 16h, and 150 mL of water was added. This mixture was extracted twice with 200 mL portions of ethyl acetate, and the combined organic layers were washed with saturated aqueous sodium chloride solution, dried over anhydrous sodium sulfate, and concentrated under reduced pressure to obtain a crude residue. The crude material was purified by column chromatography on silica gel (40 g) by using combi-flash, eluting with 20% ethyl acetate/petroleum ether to obtain N-[1-[4-bromo-2-(2-pyridyl)pyrazol-3-yl]ethyl]-N-(cyclopropylmethyl)-3,5-bis(trifluoromethyl)benzamide (620 mg, 48%) as an off-white solid. NMR (DMSO-d₆) δ 8.39 (s, 1H), 8.12 (s, 1H), 8.03 (s, 1H), 7.86 (m, 1H), 7.60 (s, 2H), 7.45 (m, 2H), 6.28 (m, 1H), 3.06 (m, 2H), 1.86 (d, 3H), 0.66 (m, 3H), 0.35 (m, 2H).

### Step G: Preparation of N-[1-[4-cyano-2-(2-pyridyl)pyrazol-3-yl]ethyl]-N-(cyclopropylmethyl)-3,5-bis(trifluoromethyl)benzamide

To a solution of N-[1-[4-bromo-2-(2-pyridyl)pyrazol-3-yl]ethyl]-N-(cyclopropylmethyl)-3,5-bis(trifluoromethyl)benzamide (300 mg, 0.53 mmol, 1.0 eq) in dry N,N-dimethylformamide (3 mL) was added copper(I) cyanide (71 mg, 0.8 mmol, 1.5 eq), in a sealed tube at ambient temperature. The resulting reaction mixture was stirred at 160° C. for 24h. Water (50 mL) was added, and the mixture was extracted twice with 200 mL portions of ethyl acetate. The combined organic layers were washed with saturated aqueous sodium chloride solution, dried over anhydrous sodium sulfate, and concentrated under reduced pressure to obtain a crude residue. The crude material was purified by preparative high-performance liquid chromatography to afford N-[1-[4-cyano-2-(2-pyridyl)pyrazol-3-yl]ethyl]-N-(cyclopropylmethyl)-3,5-bis(trifluoromethyl)benzamide (113 mg, 41%) as an off-white solid. ¹H NMR (DMSO-d₆) δ 8.49 (s, 1H), 8.37 (s, 1H), 8.18 (m, 1H), 8.12 (m, 1H), 7.82 (s, 1H), 7.68 (s, 2H), 7.56 (m, 1H), 6.26 (m, 1H), 2.96 (m, 2H), 1.90 (d, 3H), 1.23 (m, 3H), 0.34 (m, 2H).

### Synthesis Example 17

### Preparation of N-[1-(4-cyano-5-methoxy-2-pyrimidin-2-yl-pyrazol-3-yl)ethyl]-3,5-bis(trifluoromethyl)benzamide (Compound 82)

### Step A: Preparation of 2-(3-methoxypyrazol-1-yl)pyrimidine

A mixture of cesium carbonate (30.2 g, 92.7 mmol, 1.2 eq), 3-methoxy-1H-pyrazole (7.58 g, 77.3 mmol, 1 eq), 2-chloropyrimidine (8.85 g, 77.3 mmol, 1 eq) and N,N-dimethylformamide (155 mL) was heated to 120° C. and stirred for 16 h. The reaction was cooled to room temperature and diluted with water (150 mL). Toluene (150 mL) was added, and the mixture was concentrated under reduced pressure on a rotary evaporator. This procedure was repeated, then the remaining aqueous phase was extracted twice with ethyl acetate (2×150 mL). The combined organic layers were dried over magnesium sulfate, filtered, and concentrated onto Celite under reduced pressure. The residue was purified by combi-flash chromatography, eluting with a gradient of 50-70% ethyl acetate in hexanes to obtain the desired product, 2-(3-methoxypyrazol-1-yl)pyrimidine (9.5 g, 70%). ¹H NMR (500 MHz, chloroform-d) δ ppm 4.08 (s, 3H) 5.97 (d, J=2.90 Hz, 1H) 7.08 (t, J=4.81 Hz, 1H) 8.42 (d, J=2.90 Hz, 1H) 8.68 (d, J=4.88 Hz, 2H). LCMS: m/z: 177 [M+H]+

### Step B: Preparation of 2-(5-iodo-3-methoxy-pyrazol-1-yl)pyrimidine

To a cooled solution (T<0° C.) of 2-(3-methoxypyrazol-1-yl)pyrimidine (7.5 g, 42.6 mmol, 1 eq) in tetrahydrofuran (113 mL) was added 2,2,6,6-tetramethylpiperidinylzinc chloride lithium chloride complex, 17 w/v % solution in tetrahydrofuran (13.6 g, 79.9 mL, 17 w/v %, 47.9 mmol, 1.125 eq.) dropwise by keeping temperature, T<5° C. The reaction mixture was allowed to warm to room temperature and stirred for 6 h. After this time, was added iodine (13.56 g, 53.43 mmol, 1.255 eq) at room temperature and the reaction was stirred at room temperature for 16 h. Upon reaction completion, the reaction was cooled to 0° C., diluted with saturated aqueous ammonium chloride solution (200 mL) and extracted three times with ethyl acetate (3×200 mL). The combined organic layers were washed with saturated aqueous sodium bisulfite solution (200 mL) and saturated aqueous sodium chloride solution (100 mL). The organic layer was dried over magnesium sulfate, filtered, and and concentrated onto Celite under reduced pressure. The residue was purified by combi-flash chromatography, eluting with a gradient of 60-100% ethyl acetate in hexanes to obtain the desired product, 2-(5-iodo-3-methoxy-pyrazol-1-yl)pyrimidine (11.98 g, 93%). ¹H NMR (500 MHz, chloroform-d) δ ppm 4.06 (s, 3H) 6.28 (s, 1H) 7.25 (t, J=1.00 Hz, 1H) 8.81 (d, J=4.88 Hz, 2H). LCMS: m/z: 303 [M+H]⁺

### Step C: Preparation of 1-(5-methoxy-2-pyrimidin-2-yl-pyrazol-3-yl)ethanone

A flask with 2-(5-iodo-3-methoxypyrazol-1-yl)pyrimidine (7.3 g, 24.2 mmol, 1 eq) was evacuated and backfilled with nitrogen twice followed by the addition of anhydrous toluene (28 mL) via syringe. The solution was degassed with nitrogen for 8-10 min and then charged with tributyl(1-ethoxyvinyl)stannane (9.60 g, 8.98 mL, 1.069 g/mL, 26.6 mmol, 1.1 eq) and bis(triphenylphosphine)palladium(II) dichloride (1.696 g, 2.42 mmol, 0.1 eq). The reaction was refluxed for 16 h at 92-95° C. The reaction was cooled to room temperature and was charged with 2 M aqueous hydrogen chloride solution (36 mL), and stirred for 2 hours. The reaction was diluted with ethyl actetate (200 mL) and water (200 mL). After the separation of the layers, the organic layer was collected. The aqueous layer was extracted three times with ethyl acetate (3×100 mL). The combined organic layers were washed with saturated aqueous sodium chloride solution (100 mL), dried over magnesium sulfate, filtered, and concentrated onto Celite. The residue was purified by combi-flash chromatography, eluting with a gradient of 40-70% ethyl acetate in hexanes to obtain the desired product, 1-(5-methoxy-2-pyrimidin-2-yl-pyrazol-3-yl)ethanone (1.62 g, 31%). ¹H NMR (500 MHz, chloroform-d) δ ppm 2.52 (s, 3H) 4.07 (s, 3H) 6.20 (s, 1H) 7.21 (t, J=4.88 Hz, 1H) 8.73 (d, J=4.88 Hz, 2H). LCMS: m/z: 219 [M+H]⁺

### Step D: Preparation of N-[1-(5-methoxy-2-pyrimidin-2-yl-pyrazol-3-yl)ethylidene]-2-methyl-propane-2-sulfinamide

A mixture of 1-[5-methoxy-2-(pyrimidin-2-yl)pyrazol-3-yl]ethanone (1.88 g, 8.62 mmol, 1 eq) and tert-butanesulfinamide (1.149 g, 9.48 mmol, 1.1 equiv.) in anhydrous toluene (51 mL) was degassed with nitrogen for 5 minutes. To this solution, was added titanium isopropoxide (4.897 g, 5.10 mL, 0.96 g/mL, 17.23 mmol, 2 eq) via syringe. The reaction mixture was refluxed for 16 h at 105-110° C., then cooled to room temperature and poured rapidly into stirring saturated aqueous sodium chloride solution (100 mL). The reaction mixture was diluted with ethyl acetate (100 mL) and stirred for 10 minutes. The mixture was then filtered through a pad of celite followed by washing the filter cake with ethyl acetate (50 mL). The layers were separated, the organic layer was collected, and the aqueous layer was extracted twice with ethyl acetate (2×50 mL). The combined organic layers were dried on magnesium sulfate, filtered, and concentrated onto Celite. The residue was purified by combi-flash chromatography, eluting with a gradient of 0-100% ethyl acetate in hexanes to obtain the desired product, E and Z mixture of N-[1-(5-methoxy-2-pyrimidin-2-yl-pyrazol-3-yl)ethylidene]-2-methyl-propane-2-sulfinamide (1.41 g, 51%). 1H NMR (500 MHz, chloroform-d) δ ppm 1.19 (s, 27H) 2.52 (s, 3H) 2.63 (s, 6H) 4.07 (s, 6H) 4.08-4.09 (m, 3H) 5.96-6.04 (m, 1H) 6.11 (s, 2H) 7.08-7.14 (m, 1H) 7.17 (t, J=4.73 Hz, 2H) 8.67 (br d, J=4.12 Hz, 2H) 8.72 (d, J=4.73 Hz, 4H). LCMS: m/z: 322 [M+H]⁺

### Step E: Preparation of N-[1-(5-methoxy-2-pyrimidin-2-yl-pyrazol-3-yl)ethyl]-2-methyl-propane-2-sulfinamide

To a solution of N-[1-(5-methoxy-2-pyrimidin-2-yl-pyrazol-3-yl)ethylidene]-2-methyl-propane-2-sulfinamide (1.21 g, 3.765 mmol, 1 eq) in methanol (75 mL) at 0° C., was added sodium borohydride (0.285 g, 7.53 mmol, 2 eq) portionwise. The reaction was allowed to warm to room temperature after gas evolution stopped, and stirred for 2 hours. The reaction was cooled to 0° C. and quenched with saturated ammonium chloride (100 mL). The solution was then concentrated under reduced pressure and the residue was partitioned between water (100 mL) and ethyl acetate (100 mL). The organic layer was collected and the aqueous layer was washed twice with ethyl acetate (2×100 mL). The combined organic layers were washed with saturated aqueous sodium chloride solution (50 mL), dried over magnesium sulfate, filtered, and concentrated under reduced pressure to obtain the product, N-[1-(5-methoxy-2-pyrimidin-2-yl-pyrazol-3-yl)ethyl]-2-methyl-propane-2-sulfinamide (1.22 g). The product was used in the next step without further purification. LCMS: m/z: 324 [M+H]⁺

Step F: Preparation of 1-(5-methoxy-2-pyrimidin-2-yl-pyrazol-3-yl)ethylammonium chloride

To a solution of N-[1-(5-methoxy-2-pyrimidin-2-yl-pyrazol-3-yl)ethyl]-2-methyl-propane-2-sulfinamide, prepared as in Step E (128 mg, 0.396 mmol, 1 equiv.), in 1,4-dioxane (0.64 mL) at 0° C., was added hydrogen chloride solution in dioxanes (0.014 g, 0.099 mL, 4 M, 0.396 mmol, 1 equiv.) via syringe. The reaction mixture was stirred at room temperature for an additional 30 minutes. The solid was collected by filtration and was washed with dioxanes (2-3 mL) and dried under reduced pressure to afford the product, 1-(5-methoxy-2-pyrimidin-2-yl-pyrazol-3-yl)ethylammonium chloride (0.07 g, 69%). ¹H NMR (600 MHz, DMSO-d₆) δ ppm 1.60 (d, J=6.90 Hz, 3H) 3.90 (s, 3H) 5.19 (dt, J=12.03, 6.06 Hz, 1H) 6.39 (s, 1H) 7.46 (t, J=4.81 Hz, 1H) 8.48 (br s, 3H) 8.86 (d, J=4.90 Hz, 2H). LCMS: m/z: 220 [M+H]⁺ (free amine).

### Step G: Preparation of N-[1-(5-methoxy-2-pyrimidin-2-yl-pyrazol-3-yl)ethyl]-3,5-bis(trifluoromethyl)benzamide

To a mixture of 1-(5-methoxy-2-pyrimidin-2-yl-pyrazol-3-yl)ethylammonium chloride (0.79 g, 3.089 mmol, 1 eq) and dichloromethane (25 mL) at 0° C. was added 3,5-bis(trifluoromethyl)benzoyl chloride (1.28 g, 0.84 mL, 1.526 g/mL, 4.631 mmol, 1.5 eq) followed by dropwise addition of triethylamine (0.938 g, 1.292 mL, 0.726 g/mL, 9.2876 mmol, 3 eq). The reaction mixture was stirred at 0° C. for 10 minutes and was then allowed to warm to room temperature and stirred for 16 h. The reaction was quenched with saturated aqueous sodium bicarbonate solution (100 mL) and extracted three times with dichloromethane (3×100 mL). The combined organic layers were dried on magnesium sulfate, filtered, and concentrated onto Celite. The residue was purified by combi-flash chromatography, eluting with a gradient of 20-60% ethyl acetate in hexanes to obtain the desired product, N-[1-(5-methoxy-2-pyrimidin-2-yl-pyrazol-3-yl)ethyl]-3,5-bis(trifluoromethyl)benzamide, a compound of this invention (0.90 g, 63%). ¹H NMR (500 MHz, chloroform-d) δ ppm 1.56 (d, J=7.02 Hz, 3H) 4.06 (s, 3H) 5.98-6.06 (m, 1H) 6.07 (s, 1H) 7.24 (t, J=1.00 Hz, 1H) 8.01 (s, 1H) 8.22 (s, 2H) 8.46 (br d, J=7.93 Hz, 1H) 8.80 (d, J=4.88 Hz, 2H). LCMS: m/z: 460 [M+H]⁺

### Step H: Preparation of N-[1-(4-iodo-5-methoxy-2-pyrimidin-2-yl-pyrazol-3-yl)ethyl]-3,5-bis(trifluoromethyl)benzamide

To a solution of N-[1-(5-methoxy-2-pyrimidin-2-yl-pyrazol-3-yl)ethyl]-3,5-bis(trifluoromethyl)benzamide (0.90 g, 1.96 mmol, 1 eq) in N,N-dimethylformamide (9 mL), was added N-iodosuccinimide (0.485 g, 2.16 mmol, 1.1 eq). The reaction mixture was heated to 80° C. and stirred for 3 hours. The reaction mixture was cooled to room temperature and partitioned between water (100 mL) and ethyl acetate (100 mL). The organic layer was collected and the aqueous layer was extracted with ethyl acetate (100 mL). The combined organic layers were washed with saturated aqueous sodium bisulfite solution (100 mL), dried on magnesium sulfate, filtered, and concentrated onto Celite. The residue was purified by combi-flash chromatography, eluting with a gradient of 30-60% ethyl acetate in hexanes to obtain the desired product, N-[1-(4-iodo-5-methoxy-2-pyrimidin-2-yl-pyrazol-3-yl)ethyl]-3,5-bis(trifluoromethyl)benzamide, a compound of this invention (0.82 g, 72%). ¹H NMR (500 MHz, chloroform-d) δ ppm 1.51 (d, J=7.32 Hz, 3H) 4.11 (s, 3H) 6.14-6.24 (m, 1H) 7.34 (t, J=1.00 Hz, 1H) 8.01 (s, 1H) 8.29 (s, 2H) 8.86 (d, J=4.73 Hz, 2H) 9.26 (br d, J=8.54 Hz, 1H).

LCMS: m/z: 586 [M+H]⁺

### Step 1 Preparation of N-[1-(4-cyano-5-methoxy-2-pyrimidin-2-yl-pyrazol-3-yl)ethyl]-3,5-bis(trifluoromethyl)benzamide

In a microwave vial with stir bar, was added N-[1-(4-iodo-5-methoxy-2-pyrimidin-2-yl-pyrazol-3-yl)ethyl]-3,5-bis(trifluoromethyl)benzamide (0.63 g, 1.08 mmol, 1 eq), zinc cyanide (0.506 g, 0.273 mL, 1.852 g/mL, 4.31 mmol, 4 eq), tris(dibenzylideneacetone)dipalladium(0) (0.099 g, 0.108 mmol, 0.1 eq) and 1,1′-ferrocenediyl-bis(diphenylphosphine) (0.119 g, 0.215 mmol, 0.2 equiv.) and anhdydrous N,N-dimethylformamide (6.3 mL) under nitrogen. The reaction mixture was irradiated under microwave at 120° C. for 30 minutes. The reaction mixture was cooled to room temperature and partitioned between water (25 mL) and ethyl acetate (25 mL). The organic layer was collected and the aqueous layer was extracted with ethyl acetate (25 mL). The combined organic layers were dried on magnesium sulfate, filtered, and concentrated onto Celite. The residue was purified by combi-flash chromatography, eluting with a gradient of 30-50% ethyl acetate in hexanes to obtain the desired product, N-[1-(4-cyano-5-methoxy-2-pyrimidin-2-yl-pyrazol-3-yl)ethyl]-3,5-bis(trifluoromethyl)benzamide, a compound of this invention (0.076 g, 15% yield) product. ¹H NMR (500 MHz, chloroform-d) δ ppm 1.80 (d, J=7.02 Hz, 3H) 4.13 (s, 3H) 6.41 (quin, J=7.17 Hz, 1H) 7.35-7.40 (m, 1H) 7.43 (br d, J=7.17 Hz, 1H) 8.03 (s, 1H) 8.32 (s, 2H) 8.90 (d, J=4.88 Hz, 2H). LCMS: m/z: 485 [M+H]⁺

### Synthesis Example 18

### N-methyl-N-[1-[4-methylsulfonyl-2-[6-(trifluoromethyl)pyrimidin-4-yl]pyrazol-3-yl]ethyl]-3,5-bis(trifluoromethyl)benzamide (Compound 125)

### Step A: Preparation of Methyl 2-(Methylamino)Propanoate Hydrogen Chloride Salt

To a stirred solution of 2-(methylamino)propanoic acid (N-methyl-DL-alanine) (21 g, 203.6 mmol) in methanol (300 mL) under nitrogen atmosphere cooled to 0° C. was slowly added thionyl chloride (21 mL, 305 mmol) at 0° C. The mixture was stirred for 15 min at 0° C. and then heated at reflux for 5 h. The reaction mixture was concentrated under reduced pressure to obtain crude methyl 2-(methylamino)propanoate hydrogen chloride (28 g, yield 90%) as an oily material which was used in the next step without purification.

¹H NMR (500 MHz, DMSO) δ 9.63 (bs, NH), 9.29 (bs, NH), 4.11-4.08 (m, 1H), 3.76 (s, 3H), 2.56-2.51 (m, 3H), 1.48 (d, J=7.0 Hz, 3H)

### Step B: Preparation of Methyl 2-[[3,5-bis(trifluoromethyl)benzoyl]-methyl amino]propanoate

To a stirred solution of 3,5-bis(trifluoromethyl)benzoic acid (45 g, 0.174 mol) in dichloromethane (250 mL) was added dimethylformamide (1 mL), and oxalyl chloride (32.6 mL, 0.383 mol) was added dropwise at 0° C. under nitrogen atmosphere. The mixture was stirred at ambient temperature for 3 h and then was concentrated under reduced pressure to obtain crude acid chloride.

In a separate dry round bottom flask, methyl 2-(methylamino)propanoate hydrogen chloride (obtained as above, 26.6 g, 0.174 mol) was dissolved in tetrahydrofuran (100 mL), N,N-diisopropylethylamine (155 mL, 0.87 mol) was added, and the acid chloride prepared above was added as a solution in tetrahydrofuran (150 mL) dropwise at 0° C. The mixture was allowed to warm to ambient temperature and stir for 16 h. The reaction mixture was poured into ice-cold water (300 mL) and extracted with ethyl acetate (200 mL×3). The combined organic phases were washed with water (100 mL) and with saturated aqueous sodium chloride solution (100 mL), dried over sodium sulfate, and concentrated under reduced pressure. The residue was subjected to flash column chromatography (0-20% ethyl acetate in petroleum ether) to obtain methyl 2-[[3,5-bis(trifluoromethyl)benzoyl]-methyl-amino]propanoate (45 g, yield 62%) as a yellow liquid.

LCMS (ESI) [M+H]+: 358.17. ¹H NMR (500 MHz, CDCl3) δ 7.94-7.91 (m, 3H), 5.28-5.24 (m, 1H), 3.79 (s, 3H), 2.94 (s, 3H), 1.56 (d, 3H)

### Step C: Preparation of N-methyl-N-(1-methyl-3-methylsulfonyl-2-oxo-propyl)-3,5-bis (trifluoromethyl)benzamide

To a stirred solution of dimethyl sulfone (9.85 g, 104.7 mmol) in tetrahydrofuran (220 mL) was added n-butyl lithium (2.5M in hexanes, 38 mL, 95 mmol) at 0° C. under nitrogen atmosphere and stirred at 0° C. for 20 min. This mixture was cooled to −78° C. and a solution of methyl 2-[[3,5-bis(trifluoromethyl)benzoyl]-methyl-amino]propanoate (17 g, 47.6 mmol) in dry tetrahydrofuran (50 mL) was added dropwise at −78° C. and allowed to warm slowly to 0° C. over 1 h. The reaction mixture was poured into cold aqueous 1N hydrogen chloride solution (300 mL) and extracted with ethyl acetate (200 mL×3) The combined organic phases were washed with water (100 mL), saturated aqueous sodium chloride solution (100 mL), dried over anhydrous sodium sulfate, and concentrated under reduced pressure. The residue was subjected to flash column chromatography (0-50% ethyl acetate in petroleum ether) to obtain N-methyl-N-(4-(methylsulfonyl)-3-oxobutan-2-yl)-3,5-bis(trifluoromethyl)benzamide (18.5 g, yield 92%) as a white solid.

LCMS (ESI) [M+H]⁺: 420.10 ¹H NMR (400 MHz, CDCl3) δ 7.98 (bs, 1H), 7.92 (bs, 2H), 4.71 (q, J=6.8 Hz, 1H), 4.28-4.19 (m, 2H), 3.12 (s, 3H), 3.06 (s, 3H), 1.56 (d, J=7.2 Hz, 3H)

### Step a: Preparation of [6-(Trifluoromethyl)pyrimidin-4-yl]hydrazine

To a stirred solution of 4-chloro-6-(trifluoromethyl)pyrimidine (1 g, 5.4 mmol) in ethanol (10 mL) were added hydrazine monohydrate (0.52 mL, 10.8 mmol) and triethylamine (0.83 mL, 5.94 mmol) at 25° C. The reaction mixture stirred at 100° C. for 5 h, and was then concentrated under reduced pressure. The crude residue obtained was diluted with chloroform (20 mL), filtered through celite, and concentrated to a solid. This solid was triturated with diethyl ether (5 mL) and n-pentane (10 mL). The resultant solid was dried under vacuum to obtain the title material (1g, Crude) as an off-white solid.

LCMS (ESI) [M+H]⁺: 178.91. ¹H NMR (400 MHz, CDCl3) δ 8.64 (s, 1H), 7.16 (bs, 1H), 6.93 (s, 1H), 3.94 (bs, 2H)

### Steps D & E: Preparation of N-methyl-N-[1-[4-methylsulfonyl-2-[6-(trifluoromethyl) pyrimidin-4-yl]pyrazol-3-yl]ethyl]-3,5-bis(trifluoromethyl)benzamide

To a stirred solution of N-methyl-N-(4-(methylsulfonyl)-3-oxobutan-2-yl)-3,5-bis(trifluoromethyl)benzamide (500 mg, 1.19 mmol) in dichloromethane (10 mL) was added dimethylformamide dimethylacetal (0.46 mL, 3.57 mmol) at ambient temperature under nitrogen atmosphere. The reaction mixture was stirred at ambient temperature for 16 h and then concentrated under reduced pressure to obtain a crude residue. This residue was dissolved in ethanol (5 mL) and acetic acid (1.5 mL) and [6-(trifluoromethyl)pyrimidin-4-yl]hydrazine (1g as obtained above in Step 1a)) were added, and the mixture was heated at 100° C. for 4 h. The reaction mixture was concentrated under reduced pressure, and the obtained crude material was treated with saturated aqueous sodium bicarbonate solution to neutral pH (30 mL) followed by extraction with dichloromethane (30 mL×3). The combined organic phases were washed with water (30 mL) and saturated aqueous sodium chloride solution (30 mL), dried over sodium sulfate. The organic phase was concentrated under reduced pressure and the residue purified by column chromatography, eluting with 50% ethyl acetate in petroleum ether to obtain N-methyl-N-[1-[4-methylsulfonyl-2-[6-(trifluoromethyl)pyrimidin-4-yl]pyrazol-3-yl]ethyl]-3,5-bis(trifluoromethyl)benzamide, a compound of this invention (300 mg, yield 42%) as a white solid.

LCMS (ESI) [M+H]⁺: 590.19 ¹H NMR (400 MHz, CDCl3) δ 9.30 (s, 1H), 8.32 (s, 1H), 8.06 (s, 1H), 7.88 (bs, 1H), 7.67 (bs, 2H), 6.45 (q, J=7.2 Hz, 1H), 3.29 (s, 3H), 3.20 (s, 3H), 2.02 (d, J=7.2 Hz, 3H)

By the procedures described herein together with methods known in the art, the following compounds can be prepared. The following abbreviations are used in the Tables which follow: Me means methyl, Et means ethyl, i-Pr means isopropyl, i-Bu means isobutyl, c-Pr means cyclopropyl and OMe means methoxy.

TABLE 1

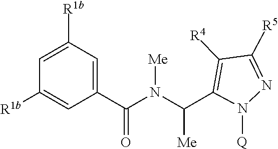

Q is Q-1, R⁴ is CN and R⁵ is NH₂

| R^{1a} | R^{1b} | R^{1a} | R^{1b} |
|---|---|---|---|
| Cl | Cl | CF₃ | OSO₂Me |
| Cl | Br | CF₃ | OSO₂Et |
| Cl | CF₃ | CF₃ | OSO₂CF₃ |
| Cl | OCF₃ | OCF₃ | OCF₃ |
| Cl | SCF₃ | OCF₃ | SCF₃ |
| Cl | 1-c-Pr | OCF₃ | 1-c-Pr |
| Cl | 1-c-PrCN | OCF₃ | 1-c-PrCN |
| Cl | SO₂Me | OCF₃ | SO₂Me |
| Cl | SO₂CF₃ | OCF₃ | SO₂CF₃ |
| Cl | OSO₂Me | OCF₃ | OSO₂Me |
| Cl | OSO₂Et | OCF₃ | OSO₂Et |
| Cl | OSO₂CF₃ | OCF₃ | OSO₂CF₃ |
| Br | Br | SCF₃ | SCF₃ |
| Br | CF₃ | SCF₃ | 1-c-Pr |
| Br | OCF₃ | SCF₃ | 1-c-PrCN |
| Br | SCF₃ | SCF₃ | SO₂Me |
| Br | 1-c-Pr | SCF₃ | SO₂CF₃ |
| Br | 1-c-PrCN | SCF₃ | OSO₂Me |
| Br | SO₂Me | SCF₃ | OSO₂Et |
| Br | SO₂CF₃ | SCF₃ | OSO₂CF₃ |
| Br | OSO₂Me | OSO₂Me | F |
| Br | OSO₂Et | OSO₂Me | 1-c-Pr |
| Br | OSO₂CF₃ | OSO₂Me | 1-c-PrCN |
| CF₃ | CF₃ | OSO₂Et | F |
| CF₃ | OCF₃ | OSO₂Et | 1-c-Pr |
| CF₃ | SCF₃ | OSO₂Et | 1-c-PrCN |
| CF₃ | 1-c-Pr | OSO₂CF₃ | F |
| CF₃ | 1-c-PrCN | OSO₂CF₃ | 1-c-Pr |
| CF₃ | SO₂Me | OSO₂CF₃ | 1-c-PrCN |
| CF₃ | SO₂CF₃ | | |

Table 2 is identical to Table 1, except that R⁴ is NO₂. Table 3 is identical to Table 1, except that R⁴ is Me. Table 4 is identical to Tables 1 through 3, except that R⁵ is NHMe. Table 5 is identical to Tables 1 through 3, except that R⁵ is OMe. Table 6 is identical to Tables 1 through 3, except that R⁵ s SMe.
Table 7 is identical to Tables 1 through 3, except that R⁵ is Cl.
Table 8 is identical to Tables 1 through 7, except that Q is Q-2.
Table 9 is identical to Tables 1 through 7, except that Q is Q-3.
Table 10 is identical to Tables 1 through 7, except that Q is Q-4.
Table 11 is identical to Tables 1 through 7, except that Q is Q-5.
Table 12 is identical to Tables 1 through 7, except that Q is Q-6.
Table 13 is identical to Tables 1 through 7, except that Q is Q-7.
Table 14 is identical to Tables 1 through 7, except that Q is Q-8.
Table 15 is identical to Tables 1 through 7, except that Q is Q-9.
Table 16 is identical to Tables 1 through 7, except that Q is Q-10.
Table 17 is identical to Tables 1 through 7, except that Q is Q-11.
Table 18 is identical to Tables 1 through 7, except that Q is Q-12.
Table 19 is identical to Tables 1 through 7, except that Q is Q-13.
Table 20 is identical to Tables 1 through 7, except that Q is Q-14.
Table 21 is identical to Tables 1 through 7, except that Q is Q-15.
Table 22 is identical to Tables 1 through 7, except that Q is Q-16.
Table 23 is identical to Tables 1 through 7, except that Q is Q-17.

TABLE 24

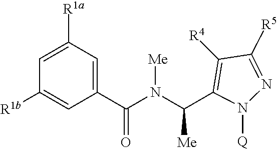

Table 24 is identical to Tables 1 through 23, except that the structure shown under the heading “Table 1” is replaced by the structure shown above.

TABLE 25

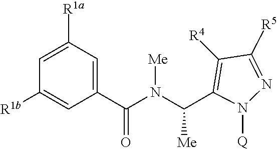

Table 25 is identical to Tables 1 through 23, except that the structure shown under the heading “Table 1” is replaced by the structure shown above.

TABLE 26

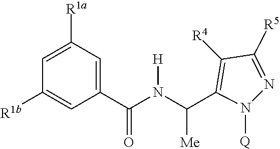

Q is Q-1, R⁴ is CN and R⁵ is NH₂

| R^{1a} | R^{1b} | R^{1a} | R^{1b} |
|---|---|---|---|
| Cl | Cl | CF₃ | OSO₂Me |
| Cl | Br | CF₃ | OSO₂Et |
| Cl | CF₃ | CF₃ | OSO₂CF₃ |
| Cl | OCF₃ | OCF₃ | OCF₃ |
| Cl | SCF₃ | OCF₃ | SCF₃ |
| Cl | 1-c-Pr | OCF₃ | 1-c-Pr |
| Cl | 1-c-PrCN | OCF₃ | 1-c-PrCN |
| Cl | SO₂Me | OCF₃ | SO₂Me |
| Cl | SO₂CF₃ | OCF₃ | SO₂CF₃ |
| Cl | OSO₂Me | OCF₃ | OSO₂Me |
| Cl | OSO₂Et | OCF₃ | OSO₂Et |
| Cl | OSO₂CF₃ | OCF₃ | OSO₂CF₃ |
| Br | Br | SCF₃ | SCF₃ |
| Br | CF₃ | SCF₃ | 1-c-Pr |
| Br | OCF₃ | SCF₃ | 1-c-PrCN |
| Br | SCF₃ | SCF₃ | SO₂Me |
| Br | 1-c-Pr | SCF₃ | SO₂CF₃ |
| Br | 1-c-PrCN | SCF₃ | OSO₂Me |
| Br | SO₂Me | SCF₃ | OSO₂Et |
| Br | SO₂CF₃ | SCF₃ | OSO₂CF₃ |
| Br | OSO₂Me | OSO₂Me | F |
| Br | OSO₂Et | OSO₂Me | 1-c-Pr |
| Br | OSO₂CF₃ | OSO₂Me | 1-c-PrCN |
| CF₃ | CF₃ | OSO₂Et | F |
| CF₃ | OCF₃ | OSO₂Et | 1-c-Pr |
| CF₃ | SCF₃ | OSO₂Et | 1-c-PrCN |
| CF₃ | 1-c-Pr | OSO₂CF₃ | F |
| CF₃ | 1-c-PrCN | OSO₂CF₃ | 1-c-Pr |
| CF₃ | SO₂Me | OSO₂CF₃ | 1-c-PrCN |
| CF₃ | SO₂CF₃ | | |

Table 27 is identical to Table 26, except that R⁴ is NO₂. Table 28 is identical to Table 26, except that R⁴ is Me.
Table 29 is identical to Tables 26 through 28, except that R⁵ is NHMe.
Table 30 is identical to Tables 26 through 28, except that R⁵ is OMe.
Table 31 is identical to Tables 26 through 28, except that R⁵ is SMe.
Table 32 is identical to Tables 26 through 28, except that R⁵ is Cl.
Table 33 is identical to Tables 26 through 32, except that Q is Q-2.
Table 34 is identical to Tables 26 through 32, except that Q is Q-3.
Table 35 is identical to Tables 26 through 32, except that Q is Q-4.
Table 36 is identical to Tables 26 through 32, except that Q is Q-5.
Table 37 is identical to Tables 26 through 32, except that Q is Q-6.
Table 38 is identical to Tables 26 through 32, except that Q is Q-7.
Table 39 is identical to Tables 26 through 32, except that Q is Q-8.
Table 40 is identical to Tables 26 through 32, except that Q is Q-9.
Table 41 is identical to Tables 26 through 32, except that Q is Q-10.
Table 42 is identical to Tables 26 through 32, except that Q is Q-11.
Table 43 is identical to Tables 26 through 32, except that Q is Q-12.
Table 44 is identical to Tables 26 through 32, except that Q is Q-13.
Table 45 is identical to Tables 26 through 32, except that Q is Q-14.
Table 46 is identical to Tables 26 through 32, except that Q is Q-15.
Table 47 is identical to Tables 26 through 32, except that Q is Q-16.
Table 48 is identical to Tables 26 through 32, except that Q is Q-17.

TABLE 49

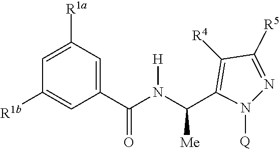

Table 49 is identical to Tables 26 through 48, except that the structure shown under the heading “Table 49” is replaced by the structure shown above.

TABLE 50

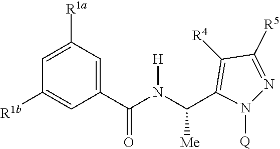

Table 50 is identical to Tables 26 through 48, except that the structure shown under the heading “Table 50” is replaced by the structure shown above.

Specific compounds of Formula 1, prepared by the methods and variations as described in preceding Schemes 1-13 and Synthesis Examples 1-18, are shown in the Index Table below. The following abbreviations may be used: Cmpd means Compound, t is tertiary, c is cyclo, Me is methyl, Et is ethyl and Ph is phenyl. The abbreviation “Ex.” stands for “Example” and is followed by a number indicating in which Synthesis Example the compound is prepared.

Melting point data (MP) is reported as a temperature range (for example, 122-126). Mass spectral data (MS) is reported as a single numerical value (for example, 542). For mass spectral data (AP⁺ (M+1)), the numerical value reported is the molecular weight of the parent molecular ion (M) formed by addition of H⁺ (molecular weight of 1) to the molecule to give a M+1 peak observed by mass spectrometry using atmospheric pressure chemical ionization (AP⁺). The alternate molecular ion peaks (e.g., M+2 or M+4) that occur with compounds containing multiple halogens are not reported.

INDEX TABLE A

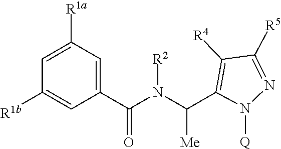

| | | | | | | | MP/ |
|---|---|---|---|---|---|---|---|
| Cmpd. | | | | | | | MS data |
| No. | R^{1a} | R^{1b} | R² | R⁴ | R⁵ | Q | (M + 1) |
| 1 | Cl | CF₃ | CH₂c- | CN | H | Q-18 | 135-138° C. |
| | | | Pr | | | | |
| 2 | F | CF₃ | CH₂c- | CN | H | Q-18 | 123-126° C. |
| | | | Pr | | | | |
| 3 | Cl | CF₃ | CH₂c- | Br | H | Q-18 | 93-96° C. |
| | | | Pr | | | | |
| 4 | CF₃ | F | CH₂c- | H | H | Q-18 | 433.6 |
| | | | Pr | | | | |
| 5 | CF₃ | CF₃ | CH₂c- | Br | H | Q-18 | 113-116° C. |
| | | | Pr | | | | |
| 6 | F | CF₃ | CH₂C- | Br | H | Q-18 | 511.6 |
| | | | Pr | | | | |
| 7 | CF₃ | CF₃ | CH₂c- | CN | H | Q-18 | 171-175° C. |
| | | | Pr | | | | |
| 8 | CF₃ | CF₃ | CH₂c- | H | H | Q-18 | 483.6 |
| | | | Pr | | | | |
| 9 | CF₃ | CF₃ | Me | H | NO₂ | Q-19 | 174-177° C. |
| 10 | CF₃ | CF₃ | Me | I | NH₂ | Q-19 | 662/669 |
| 11 | CF₃ | CF₃ | Me | H | NH₂ | Q-19 | 566/568 |
| 12 | CF₃ | CF₃ | Me | CN | NHMe | Q-2 | 522 |
| 13 | CF₃ | CF₃ | Me | CN | N(Me)₂ | Q-2 | 536 |
| 14 | CF₃ | CF₃ | Me | CN | NH₂ | Q-2 | 191-194° C. |
| 15 | CF₃ | CF₃ | Me | H | NHC(O) | Q-19 | 578/580 |
| | | | | | Me | | |
| 16 | CF₃ | CF₃ | Me | NO₂ | H | Q-1 | 489 |
| 17 | CF₃ | CF₃ | Me | CN | NHMe | Q-20 | 187-190° C. |
| 18 | CF₃ | CF₃ | Me | CN | N(Me)₂ | Q-20 | 117-120° C. |
| 19 | CF₃ | CF₃ | Me | H | N(Me)₂ | Q-21 | 508 M+1 |
| 20 | CF₃ | CF₃ | Me | Br | H | Q-19 | 149-151° C. |
| 21 | CF₃ | CF₃ | Me | H | NHC(O) | Q-20 | 613 |
| | | | | | Me | | |
| 22 | CF₃ | CF₃ | Me | H | NH₂ | Q-22 | 544 |
| 23 | CF₃ | CF₃ | Me | I | NH₂ | Q-21 | 642 |
| 24 | CF₃ | CF₃ | Me | CN | NH₂ | Q-21 | 229-233° C. |
| 25 | CF₃ | CF₃ | Me | CN | NH₂ | Q-22 | 193-196° C. |
| 26 | CF₃ | CF₃ | Me | CN | NH₂ | Q-20 | 211-214° C. |
| 27 | CF₃ | CF₃ | Me | H | NHC(O) | Q-22 | 558 |
| | | | | | Me | | |
| 28 | CF₃ | CF₃ | Me | H | NHC(O) | Q-22 | 544 |
| | | | | | Me | | |
| 29 | CF₃ | CF₃ | Me | CN | H | Q-2 | 541 |
| 30 | CF₃ | CF₃ | Me | H | NH₂ | Q-21 | 516 |
| 31 | CF₃ | CF₃ | Me | H | N(Me)₂ | Q-21 | 112-115° C. |
| 35 | CF₃ | CF₃ | Me | CN | NH₂ | Q-22 | 527 |
| 41 | CF₃ | Cl | Me | CN | H | Q-2 | 85-87° C. |
| 42 | CF₃ | CF₃ | Me | NO₂ | Cl | Q-19 | 134-137° C. |
| 43 | CF₃ | Cl | Me | Br | H | Q-19 | 137-140° C. |
| 44 | CF₃ | CF₃ | H | CN | H | Q-1 | 137-140° C. |
| 45 | Cl | CF₃ | Me | Br | H | Q-19 | 146-148° C. |
| 46 | CF₃ | OS(O)₂ | Me | CN | H | Q-2 | 145-148° C. |
| | | CF₃ | | | | | |
| 47 | CF₃ | CF₃ | Me | c-Pr | Cl | Q-2 | 148-151° C. |
| 48 | CF₃ | CF₃ | Me | H | Cl | Q-2 | 150-153° C. |
| 49 | CF₃ | CF₃ | Me | CH₂ | Cl | Q-2 | 151-154° C. |
| | | | | CH₃ | | | |
| 50 | CF₃ | CF₃ | H | CF₃ | H | Q-2 | 185-187° C. |
| 51 | CF₃ | CF₃ | H | Br | NH₂ | Q-2 | 157-160° C. |
| 52 | CF₃ | CF₃ | Me | NO₂ | H | Q-11 | 161-164° C. |
| 53 | Cl | Cl | H | Br | NH₂ | Q-2 | 162-165° C. |
| 54 | CF₃ | CF₃ | Me | CN | Cl | Q-2 | 165-168° C. |
| 55 | CF₃ | CF₃ | Me | NO₂ | H | Q-20 | 167-170° C. |
| 56 | CF₃ | CF₃ | Me | Br | Cl | Q-2 | 170-173° C. |
| 57 | CF₃ | CF₃ | Me | Br | Cl | Q-19 | 173-176° C. |
| 58 | CF₃ | CF₃ | Me | S(O)₂ | H | Q-13 | 175-178° C. |
| | | | | Me | | | |
| 59 | CF₃ | CF₃ | H | Cl | NH₂ | Q-2 | 177-180° C. |
| 60 | CF₃ | CF₃ | Me | H | Cl | Q-19 | 182-185° C. |
| 61 | Cl | Cl | H | CF₃ | H | Q-2 | 185-187° C. |
| 62 | CF₃ | CF₃ | Me | NO₂ | H | Q-21 | 186-189° C. |
| 63 | Cl | Cl | H | C(O) | H | Q-2 | 192-195° C. |
| | | | | OMe | | | |
| 64 | CF₃ | CF₃ | Me | CN | CN | Q-2 | 195-197° C. |
| 65 | Cl | Cl | H | Cl | H | Q-2 | 201-204° C. |
| 66 | CF₃ | OH | Me | CN | H | Q-3 | 206-209° C. |
| 67 | CF₃ | CF₃ | H | Cl | H | Q-2 | 207-210° C. |
| 68 | CF₃ | CF₃ | Me | NO₂ | Me | Q-19 | 212-215° C. |
| 69 | Cl | Cl | H | Cl | NH₂ | Q-2 | 217-220° C. |
| 70 | CF₃ | CF₃ | Me | S(O)₂ | H | Q-1 | 227-230° C. |
| 71 | OH | CF₃ | Me | CN | H | Q-2 | 229-232° C. |
| 72 | CF₃ | CF₃ | Me | NO₂ | H | Q-22 | 240-243° C. |
| 73 | CF₃ | CF₃ | Me | H | CF₃ | Q-25 | 251-254° C. |
| 74 | CF₃ | CF₃ | Me | Br | CF₃ | Q-7 | 266-269° C. |
| 75 | F | F | H | F | H | Q-1 | 348 |
| 76 | Cl | Cl | H | F | H | Q-1 | 380 |
| 77 | CF₃ | Cl | H | F | H | Q-1 | 414 |
| 78 | CF₃ | Cl | H | F | H | Q-6 | 438 |
| 79 | CF₃ | CF₃ | H | F | H | Q-1 | 448 |
| 80 | CF₃ | CF₃ | H | H | OMe | Q-1 | 460 |
| 81 | CF₃ | CF₃ | H | F | H | Q-6 | 472 |
| 82 | CF₃ | CF₃ | H | CN | OMe | Q-1 | 485 |
| 83 | CF₃ | Cl | H | F | H | Q-23 | 491 |
| 84 | CF₃ | CF₃ | H | CN | OCH₂ | Q-1 | 499 |
| | | | | | CH₃ | | |
| 85 | CF₃ | CF₃ | H | F | OCH | Q-1 | 513 |
| | | | | | (CH₃)2 | | |
| 86 | CF₃ | CF₃ | H | F | H | Q-19 | 525 |
| 87 | CF₃ | CF₃ | H | I | OMe | Q-1 | 586 |
| 88 | CF₃ | CF₃ | H | I | OCH₂ | Q-1 | 600 |
| | | | | | CH₃ | | |
| 89 | CF₃ | CF₃ | H | I | OCH | Q-1 | 614 |
| | | | | | (CH₃)₂ | | |
| 90 | CF₃ | CF₃ | H | H | OCH | Q-1 | 488 |
| | | | | | (CH₃)₂ | | |
| 91 | CF₃ | CF₃ | H | H | CH₂ | Q-1 | 474 |
| | | | | | CH₃ | | |
| 92 | CF₃ | CF₃ | Me | NO₂ | Cl | Q-2 | 547 |
| 93 | CF₃ | CF₃ | Me | I | Cl | Q-2 | 628 |
| 94 | CF₃ | CF₃ | Me | CN | Cl | Q-26 | 559 |
| 95 | CF₃ | CF₃ | Me | CN | CF₃ | Q-3 | 579 |
| 96 | CF₃ | CF₃ | Me | NO₂ | H | Q-1 | 489 |
| 97 | CF₃ | CF₃ | H | H | H | Q-19 | 507 |
| 98 | Cl | Cl | H | Cl | NH₂ | Q-19 | 488 |
| 99 | CF₃ | CF₃ | H | H | NO₂ | Q-19 | 552 |
| 100 | CF₃ | CF₃ | H | H | NH₂ | Q-19 | 522 |
| 101 | Cl | Cl | H | Cl | H | Q-19 | 473 |
| 102 | Cl | Cl | H | H | H | Q-19 | 439 |
| 103 | CF₃ | CF₃ | H | H | NH₂ | Q-2 | 469 |
| 104 | CF₃ | CF₃ | H | I | H | Q-1 | 556 |
| 105 | CF₃ | CF₃ | H | H | H | Q-1 | 430 |
| 106 | CF₃ | CF₃ | H | Cl | H | Q-19 | 541 |
| 107 | Cl | Cl | H | H | NO₂ | Q-19 | 484 |
| 108 | Cl | Cl | H | H | NH₂ | Q-19 | 454 |
| 109 | CF₃ | CF₃ | Me | S(O)₂ | H | Q-24 | 522 |
| | | | | Me | | | |
| 110 | CF₃ | CF₃ | H | H | Cl | Q-19 | 541 |
| 111 | CF₃ | CF₃ | Me | CH═ | Cl | Q-2 | 528 |
| | | | | CH₂ | | | |
| 112 | CF₃ | CF₃ | Me | NO₂ | H | Q-14 | 489 |
| 113 | CF₃ | CF₃ | H | NO₂ | H | Q-1 | 475 |
| 114 | CF₃ | CF₃ | Me | NO₂ | H | Q-11 | 161-164° C. |
| 115 | CF₃ | CF₃ | Me | NO₂ | H | Q-21 | 186-189° C. |
| 116 | CF₃ | CF₃ | Me | NO₂ | H | Q-20 | 167-170° C. |
| 117 | CF₃ | CF₃ | Me | NO₂ | H | Q-22 | 240-243° C. |
| 118 | CF₃ | CF₃ | Me | H | Cl | Q-19 | 18-185° C. |
| 119 | CF₃ | CF₃ | Me | H | Cl | Q-2 | 150-153° C. |
| 120 | CF₃ | CF₃ | Me | H | CF₃ | Q-3 | 251-254° C. |
| 121 | CF₃ | CF₃ | Me | Br | Cl | Q-2 | 170-173° C. |
| 122 | CF₃ | CF₃ | Me | Br | Cl | Q-19 | 173-176° C. |
| 123 | CF₃ | CF₃ | Me | CN | CN | Q-2 | 195-197° C. |
| 124 | Cl | Cl | Me | Cl | NH₂ | Q-2 | 217-220° C. |
| 125 | CF₃ | CF₃ | Me | S(O)₂ | H | Q-14 | 590 |
| | | | | Me | | | |
| 124 | CF₃ | CF₃ | Me | H | H | Q-2 | 454 |

A compound of this invention will generally be used as an invertebrate pest control active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil in water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil in water emulsion, flowable concentrate and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethylphosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters alkyl and aryl benzoates, y-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically

C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modem Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all formulations are prepared in conventional ways. Compound numbers refer to compounds in Index Table A. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### EXAMPLE A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 3 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 8 | 10.0% |
| attapulgite granules (low volatile matter, 0.71/0.30 mm; | 90.0% |

U.S.S. No. 25-50 sieves)

### Example D

Extruded Pellet

| | |
|---|---|
| Compound 10 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 11 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 19 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 20 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### Example H

Fertilizer Stick

| | |
|---|---|
| Compound 33 | 2.5% |
| pyrrolidone-styrene copolymer | 4.8% |
| tristyrylphenyl 16-ethoxylate | 2.3% |
| talc | 0.8% |
| corn starch | 5.0% |
| slow-release fertilizer | 36.0% |
| kaolin | 38.0% |
| water | 10.6% |

### Example I

Suspension Concentrate

| | |
|---|---|
| compound 34 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example J

Emulsion in Water

| | |
|---|---|
| compound 39 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example K

Oil Dispersion

| | |
|---|---|
| compound 35 | 25% |
| polyoxyethylene sorbitol hexaoleate | 15% |
| organically modified bentonite clay | 2.5% |
| fatty acid methyl ester | 57.5% |

### Example L

Suspoemulsion

| | |
|---|---|
| compound 38 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

### Example M

Suspension Concentrate

| | |
|---|---|
| compound 2 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example N

Emulsion in Water

| | |
|---|---|
| compound 7 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example O

Oil Dispersion

| | |
|---|---|
| compound 30 | 25% |
| polyoxyethylene sorbitol hexaoleate | 15% |
| organically modified bentonite clay | 2.5% |
| fatty acid methyl ester | 57.5% |

### Example P

Suspoemulsion

| | |
|---|---|
| compound 31 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Compounds of this invention exhibit activity against a wide spectrum of invertebrate pests. These pests include invertebrates inhabiting a variety of environments such as, for example, plant foliage, roots, soil, harvested crops or other foodstuffs, or building structures. These pests include, for example, invertebrates feeding on foliage (including leaves, stems, flowers and fruits), seeds, wood or textile fibers, and thereby causing injury or damage to, for example, growing or stored agronomic crops, forests, greenhouse crops, ornamentals, nursery crops, stored foodstuffs or fiber products, or houses or other structures or their contents. Those skilled in the art will appreciate that not all compounds are equally effective against all growth stages of all pests.

These present compounds and compositions are thus useful agronomically for protecting field crops from phytophagous invertebrate pests, and also nonagronomically for protecting other horticultural crops and plants from phytophagous invertebrate pests. This utility includes protecting crops and other plants (i.e. both agronomic and nonagronomic) that contain genetic material introduced by genetic engineering (i.e. transgenic) or modified by mutagenesis to provide advantageous traits. Examples of such traits include tolerance to herbicides, resistance to phytophagous pests (e.g., insects, mites, aphids, spiders, nematodes, snails, plant-pathogenic fungi, bacteria and viruses), improved plant growth, increased tolerance of adverse growing conditions such as high or low temperatures, low or high soil moisture, and high salinity, increased flowering or fruiting, greater harvest yields, more rapid maturation, higher quality and/or nutritional value of the harvested product, or improved storage or process properties of the harvested products. Transgenic plants can be modified to express multiple traits. Examples of plants containing traits provided by genetic engineering or mutagenesis include varieties of corn, cotton, soybean and potato expressing an insecticidal Bacillus thuringiensis toxin such as YIELD GARD®, KNOCKOUT®, STARLINK®, BOLLGARD®, NuCOTN® and NEWLEAF®, INVICTA RR2 PRO™, and herbicide-tolerant varieties of corn, cotton, soybean and rapeseed such as ROUNDUP READY®, LIBERTY LINK®, IMI®, STS® and CLEARFIELD®, as well as crops expressing N-acetyltransferase (GAT) to provide resistance to glyphosate herbicide, or crops containing the HRA gene providing resistance to herbicides inhibiting acetolactate synthase (ALS). The present compounds and compositions may exhibit enhanced effects with traits introduced by genetic engineering or modified by mutagenesis, thus enhancing phenotypic expression or effectiveness of the traits or increasing the invertebrate pest control effectiveness of the present compounds and compositions. In particular, the present compounds and compositions may exhibit enhance effects with the phenotypic expression of proteins or other natural products toxic to invertebrate pests to provide greater-than-additive control of these pests.

Compositions of this invention can also optionally comprise plant nutrients, e.g., a fertilizer composition comprising at least one plant nutrient selected from nitrogen, phosphorus, potassium, sulfur, calcium, magnesium, iron, copper, boron, manganese, zinc, and molybdenum. Of note are compositions comprising at least one fertilizer composition comprising at least one plant nutrient selected from nitrogen, phosphorus, potassium, sulfur, calcium and magnesium. Compositions of the present invention which further comprise at least one plant nutrient can be in the form of liquids or solids. Of note are solid formulations in the form of granules, small sticks or tablets. Solid formulations comprising a fertilizer composition can be prepared by mixing the compound or composition of the present invention with the fertilizer composition together with formulating ingredients and then preparing the formulation by methods such as granulation or extrusion. Alternatively solid formulations can be prepared by spraying a solution or suspension of a compound or composition of the present invention in a volatile solvent onto a previous prepared fertilizer composition in the form of dimensionally stable mixtures, e.g., granules, small sticks or tablets, and then evaporating the solvent.

Nonagronomic uses refer to invertebrate pest control in the areas other than fields of crop plants. Nonagronomic uses of the present compounds and compositions include control of invertebrate pests in stored grains, beans and other foodstuffs, and in textiles such as clothing and carpets. Nonagronomic uses of the present compounds and compositions also include invertebrate pest control in ornamental plants, forests, in yards, along roadsides and railroad rights of way, and on turf such as lawns, golf courses and pastures. Nonagronomic uses of the present compounds and compositions also include invertebrate pest control in houses and other buildings which may be occupied by humans and/or companion, farm, ranch, zoo or other animals. Nonagronomic uses of the present compounds and compositions also include the control of pests such as termites that can damage wood or other structural materials used in buildings.

Examples of agronomic or nonagronomic invertebrate pests include eggs, larvae and adults of the order Lepidoptera, such as armyworms, cutworms, loopers, and heliothines in the family Noctuidae (e.g., pink stem borer (Sesamia inferens Walker), corn stalk borer (Sesamia nonagrioides Lefebvre), southern armyworm (Spodoptera eridania Cramer), fall armyworm (Spodoptera frugiperda J. E. Smith), beet armyworm (Spodoptera exigua Hübner), cotton leafworm (Spodoptera littoralis Boisduval), yellowstriped armyworm (Spodoptera ornithogalli Guenée), black cutworm (Agrotis ipsilon Hufnagel), velvetbean caterpillar (Anticarsia gemmatalis Hübner), green fruitworm (Lithophane antennata Walker), cabbage armyworm (Barathra brassicae Linnaeus), soybean looper (Pseudoplusia includens Walker), cabbage looper (Trichoplusia ni Hübner), tobacco budworm (Heliothis virescens Fabricius)); borers, casebearers, webworms, coneworms, cabbageworms and skeletonizers from the family Pyralidae (e.g., European corn borer (Ostrinia nubilalis Hübner), navel orangeworm (Amyelois transitella Walker), corn root webworm (Crambus caliginosellus Clemens), sod webworms (Pyralidae: Crambinae) such as sod worm (Herpetogramma licarsisalis Walker), sugarcane stem borer (Chilo infuscatellus Snellen), tomato small borer (Neoleucinodes elegantalis Guenée), green leafroller (Cnaphalocrocis medinalis), grape leaffolder (Desmia funeralis Hübner), melon worm (Diaphania nitidalis Stoll), cabbage center grub (Helluala hydralis Guenée), yellow stem borer (Scirpophaga incertulas Walker), early shoot borer (Scirpophaga infuscatellus Snellen), white stem borer (Scirpophaga innotata Walker), top shoot borer (Scirpophaga nivella Fabricius), dark-headed rice borer (Chilo polychrysus Meyrick), striped riceborer (Chilo suppressalis Walker), cabbage cluster caterpillar (Crocidolomia binotalis English)); leafrollers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck), citrus false codling moth (Cryptophlebia leucotreta Meyrick), citrus borer (Ecdytolopha aurantiana Lima), redbanded leafroller (Argyrotaenia velutinana Walker), obliquebanded leafroller (Choristoneura rosaceana Harris), light brown apple moth (Epiphyas postvittana Walker), European grape berry moth (Eupoecilia ambiguella Hübner), apple bud moth (Pandemis pyrusana Kearfott), omnivorous leafroller (Platynota stultana Walsingham), barred fruit-tree tortrix (Pandemis cerasana Hübner), apple brown tortrix (Pandemis heparana Denis & Schiffermüller)); and many other economically important lepidoptera (e.g., diamondback moth (Plutella xylostella Linnaeus), pink bollworm (Pectinophora gossypiella Saunders), gypsy moth (Lymantria dispar Linnaeus), peach fruit borer (Carposina niponensis Walsingham), peach twig borer (Anarsia lineatella Zeller), potato tuberworm (Phthorimaea operculella Zeller), spotted teniform leafminer (Lithocolletis blancardella Fabricius), Asiatic apple leafminer (Lithocolletis ringoniella Matsumura), rice leaffolder (Lerodea eufala Edwards), apple leafminer (Leucoptera scitella Zeller)); eggs, nymphs and adults of the order Blattodea including cockroaches from the families Blattellidae and Blattidae (e.g., oriental cockroach (Blatta orientalis Linnaeus), Asian cockroach (Blatella asahinai Mizukubo), German cockroach (Blattella germanica Linnaeus), brownbanded cockroach (Supella longipalpa Fabricius), American cockroach (Periplaneta americana Linnaeus), brown cockroach (Periplaneta brunnea Burmeister), Madeira cockroach (Leucophaea maderae Fabricius)), smoky brown cockroach (Periplaneta fuliginosa Service), Australian Cockroach (Periplaneta australasiae Fabr.), lobster cockroach (Nauphoeta cinerea Olivier) and smooth cockroach (Symploce pallens Stephens)); eggs, foliar feeding, fruit feeding, root feeding, seed feeding and vesicular tissue feeding larvae and adults of the order Coleoptera including weevils from the families Anthribidae, Bruchidae, and Curculionidae (e.g., boll weevil (Anthonomus grandis Boheman), rice water weevil (Lissorhoptrus oryzophilus Kuschel), granary weevil (Sitophilus granarius Linnaeus), rice weevil (Sitophilus oryzae Linnaeus)), annual bluegrass weevil (Listronotus maculicollis Dietz), bluegrass billbug (Sphenophorus parvulus Gyllenhal), hunting billbug (Sphenophorus venatus vestitus), Denver billbug (Sphenophorus cicatristriatus Fahraeus)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scarabaeidae (e.g., Japanese beetle (Popillia japonica Newman), oriental beetle (Anomala orientalis Waterhouse, Exomala orientalis (Waterhouse) Baraud), northern masked chafer (Cyclocephala borealis Arrow), southern masked chafer (Cyclocephala immaculata Olivier or C. lurida Bland), dung beetle and white grub (Aphodius spp.), black turfgrass ataenius (Ataenius spretulus Haldeman), green June beetle (Cotinis nitida Linnaeus), Asiatic garden beetle (Maladera castanea Arrow), May/June beetles (Phyllophaga spp.) and European chafer (Rhizotrogus majalis Razoumowsky)); carpet beetles from the family Dermestidae; wireworms from the family Elateridae; bark beetles from the family Scolytidae and flour beetles from the family Tenebrionidae.

In addition, agronomic and nonagronomic pests include: eggs, adults and larvae of the order Dermaptera including earwigs from the family Forficulidae (e.g., European earwig (Forficula auricularia Linnaeus), black earwig (Chelisoches morio Fabricius)); eggs, immatures, adults and nymphs of the orders Hemiptera and Homoptera such as, plant bugs from the family Miridae, cicadas from the family Cicadidae, leafhoppers (e.g. Empoasca spp.) from the family Cicadellidae, bed bugs (e.g., Cimex lectularius Linnaeus) from the family Cimicidae, planthoppers from the families Fulgoroidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, chinch bugs (e.g., hairy chinch bug (Blissus leucopterus hirtus Montandon) and southern chinch bug (Blissus insularis Barber)) and other seed bugs from the family Lygaeidae, spittlebugs from the family Cercopidae squash bugs from the family Coreidae, and red bugs and cotton stainers from the family Pyrrhocoridae.

Agronomic and nonagronomic pests also include: eggs, larvae, nymphs and adults of the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)); flat mites in the family Tenuipalpidae (e.g., citrus flat mite (Brevipalpus lewisi McGregor)); rust and bud mites in the family Eriophyidae and other foliar feeding mites, dust mites in the family Epidermoptidae, follicle mites in the family Demodicidae, grain mites in the family Glycyphagidae; ticks in the family Ixodidae, commonly known as hard ticks (e.g., deer tick (Ixodes scapularis Say), Australian paralysis tick (Ixodes holocyclus Neumann), American dog tick (Dermacentor variabilis Say), lone star tick (Amblyomma americanum Linnaeus)) and ticks in the family Argasidae, commonly known as soft ticks (e.g., relapsing fever tick (Ornithodoros turicata), common fowl tick (Argas radiatus)); scab and itch mites in the families Psoroptidae, Pyemotidae, and Sarcoptidae; eggs, adults and immatures of the order Orthoptera including grasshoppers, locusts and crickets (e.g., migratory grasshoppers (e.g., Melanoplus sanguinipes Fabricius, M. differentialis Thomas), American grasshoppers (e.g., Schistocerca americana Drury), desert locust (Schistocerca gregaria Forskal), migratory locust (Locusta migratoria Linnaeus), bush locust (Zonocerus spp.), house cricket (Acheta domesticus Linnaeus), mole crickets (e.g., tawny mole cricket (Scapteriscus vicinus Scudder) and southern mole cricket (Scapteriscus borellii Giglio-Tos)); eggs, adults and immatures of the order Diptera including leafminers (e.g., Liriomyza spp. such as serpentine vegetable leafminer (Liriomyza sativae Blanchard)), midges, fruit flies (Tephritidae), frit flies (e.g., Oscinella frit Linnaeus), soil maggots, house flies (e.g., Musca domestica Linnaeus), lesser house flies (e.g., Fannia canicularis Linnaeus, F. femoralis Stein), stable flies (e.g., Stomoxys calcitrans Linnaeus), face flies, horn flies, blow flies (e.g., Chrysomya spp., Phormia spp.), and other muscoid fly pests, horse flies (e.g., Tabanus spp.), bot flies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; eggs, adults and immatures of the order Thysanoptera including onion thrips (Thrips tabaci Lindeman), flower thrips (Frankliniella spp.), and other foliar feeding thrips; insect pests of the order Hymenoptera including ants of the Family Formicidae including the Florida carpenter ant (Camponotus floridanus Buckley), red carpenter ant (Camponotus ferrugineus Fabricius), black carpenter ant (Camponotus pennsylvanicus De Geer), white-footed ant (Technomyrmex albipes fr. Smith), big headed ants (Pheidole sp.), ghost ant (Tapinoma melanocephalum Fabricius); Pharaoh ant (Monomorium pharaonis Linnaeus), little fire ant (Wasmannia auropunctata Roger), fire ant (Solenopsis geminata Fabricius), red imported fire ant (Solenopsis invicta Buren), Argentine ant (Iridomyrmex humilis Mayr), crazy ant (Paratrechina longicornis Latreille), pavement ant (Tetramorium caespitum Linnaeus), cornfield ant (Lasius alienus Förster) and odorous house ant (Tapinoma sessile Say). Other Hymenoptera including bees (including carpenter bees), hornets, yellow jackets, wasps, and sawflies (Neodiprion spp.; Cephus spp.); insect pests of the order Isoptera including termites in the Termitidae (e.g., Macrotermes sp., Odontotermes obesus Rambur), Kalotermitidae (e.g., Cryptotermes sp.), and Rhinotermitidae (e.g., Reticulitermes sp., Coptotermes sp., Heterotermes tenuis Hagen) families, the eastern subterranean termite (Reticulitermes flavipes Kollar), western subterranean termite (Reticulitermes hesperus Banks), Formosan subterranean termite (Coptotermes formosanus Shiraki), West Indian drywood termite (Incisitermes immigrans Snyder), powder post termite (Cryptotermes brevis Walker), drywood termite (Incisitermes snyderi Light), southeastern subterranean termite (Reticulitermes virginicus Banks), western drywood termite (Incisitermes minor Hagen), arboreal termites such as Nasutitermes sp. and other termites of economic importance; insect pests of the order Thysanura such as silverfish (Lepisma saccharina Linnaeus) and firebrat (Thermobia domestica Packard). Additional arthropod pests covered include: spiders in the order Araneae such as the brown recluse spider (Loxosceles reclusa Gertsch & Mulaik) and the black widow spider (Latrodectus mactans Fabricius), and centipedes in the order Scutigeromorpha such as the house centipede (Scutigera coleoptrata Linnaeus).

Examples of invertebrate pests of stored grain include larger grain borer (Prostephanus truncatus), lesser grain borer (Rhyzopertha dominica), rice weevil (Stiophilus oryzae), maize weevil (Stiophilus zeamais), cowpea weevil (Callosobruchus maculatus), red flour beetle (Tribolium castaneum), granary weevil (Stiophilus granarius), Indian meal moth (Plodia interpunctella), Mediterranean flour beetle (Ephestia kuhniella) and flat or rusty grain beetle (Cryptolestis ferrugineus).

Compounds of this invention have activity against pests in the order Lepidoptera (e.g., Alabama argillacea Hubner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenée (rice leaf roller), Crambus caliginosellus Clemens (corn root webworm), Crambus teterrellus Zincken (bluegrass webworm), Cydia pomonella Linnaeus (codling moth), Earias insulana Boisduval (spiny bollworm), Earias vittella Fabricius (spotted bollworm), Helicoverpa armigera Hubner (American bollworm), Helicoverpa zea Boddie (corn earworm), Heliothis virescens Fabricius (tobacco budworm), Herpetogramma licarsisalis Walker (sod webworm), Lobesia botrana Denis & Schiffermüller (grape berry moth), Pectinophora gossypiella Saunders (pink bollworm), Phyllocnistis citrella Stainton (citrus leafminer), Pieris brassicae Linnaeus (large white butterfly), Pieris rapae Linnaeus (small white butterfly), Plutella xylostella Linnaeus (diamondback moth), Spodoptera exigua Hubner (beet armyworm), Spodoptera litura Fabricius (tobacco cutworm, cluster caterpillar), Spodoptera frugiperda J. E. Smith (fall armyworm), Trichoplusia ni Hubner (cabbage looper) and Tuta absoluta Meyrick (tomato leafminer)).

Compounds of this invention have activity against pests in the order Homoptera including: Acyrthosiphon pisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Paaserini (rosy apple aphid), Eriosoma lanigerum Hausmann (woolly apple aphid), Hyalopterus pruni Geoffroy (mealy plum aphid), Lipaphis erysimi Kaltenbach (turnip aphid), Metopolophium dirrhodum Walker (cereal aphid), Macrosiphum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graminum Rondani (greenbug), Sitobion avenae Fabricius (English grain aphid), Therioaphis maculata Buckton (spotted alfalfa aphid), Toxoptera aurantii Boyer de Fonscolombe (black citrus aphid), and Toxoptera citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifoihi Bellows & Perring (silverleaf whitefly), Dialeurodes citri Ashmead (citrus whitefly) and Trialeurodes vaporariorum Westwood (greenhouse whitefly); Empoasca fabae Harris (potato leafhopper), Laodelphax striatellus Fallen (smaller brown planthopper), Macrolestes quadrilineatus Forbes (aster leafhopper), Nephotettix cinticeps Uhler (green leafhopper), Nephotettix nigropictus Stål (rice leafhopper), Nilaparvata lugens Stål (brown planthopper), Peregrinus maidis Ashmead (corn planthopper), Sogatella furcifera Horvath (white-backed planthopper), Sogatodes orizicola Muir (rice delphacid), Typhlocyba pomaria McAtee white apple leafhopper, Erythroneoura spp. (grape leafhoppers); Magicidada septendecim Linnaeus (periodical cicada); Icerya purchasi Maskell (cottony cushion scale), Quadraspidiotus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persimmon psylla).

Compounds of this invention have activity against pests in the order Hemiptera including: Acrosternum hilare Say (green stink bug), Anasa tristis De Geer (squash bug), Blissus leucopterus leucopterus Say (chinch bug), Cimex lectularius Linnaeus (bed bug) Corythuca gossypii Fabricius (cotton lace bug), Cyrtopeltis modesta Distant (tomato bug), Dysdercus suturellus Herrich-Schaffer (cotton stainer), Euchistus servus Say (brown stink bug), Euchistus variolarius Palisot de Beauvois (one-spotted stink bug), Graptosthetus spp. (complex of seed bugs), Halymorpha halys Stål (brown marmorated stink bug), Leptoglossus corculus Say (leaf-footed pine seed bug), Lygus lineolaris Palisot de Beauvois (tarnished plant bug), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper). Other insect orders controlled by compounds of the invention include Thysanoptera (e.g., Frankliniella occidentalis Pergande (western flower thrips), Scirthothrips citri Moulton (citrus thrips), Sericothrips variabilis Beach (soybean thrips), and Thrips tabaci Lindeman (onion thrips); and the order Coleoptera (e.g., Leptinotarsa decemlineata Say (Colorado potato beetle), Epilachna varivestis Mulsant (Mexican bean beetle) and wireworms of the genera Agriotes, Athous or Limonius).

Of note is use of compounds of this invention for controlling and combating diamondback moth (Plutella xylostella). Of note is use of compounds of this invention for controlling and combating fall armyworm (Spodoptera frugiperda). Of note is use of compounds of this invention for controlling and combating western flower thrips (Frankliniella occidentalis). Of note is use of compounds of this invention for controlling and combating potato leafhopper (Empoasca fabae). Of note is use of compounds of this invention for controlling and combating corn planthopper (Peregrinus maidis). Of note is use of compounds of this invention for controlling and combating cotton melon aphid (Aphis gossypii). Of note is use of compounds of this invention for controlling and combating green peach aphid (Myzus persicae). Of note is use of compounds of this invention for controlling and combating sweetpotato whitefly (Bemisia tabaci).

Compounds of the present invention may also be useful for increasing vigor of a crop plant.

This method comprises contacting the crop plant (e.g., foliage, flowers, fruit or roots) or the seed from which the crop plant is grown with a compound of Formula 1 in amount sufficient to achieve the desired plant vigor effect (i.e. biologically effective amount). Typically the compound of Formula 1 is applied in a formulated composition. Although the compound of Formula 1 is often applied directly to the crop plant or its seed, it can also be applied to the locus of the crop plant, i.e. the environment of the crop plant, particularly the portion of the environment in close enough proximity to allow the compound of Formula 1 to migrate to the crop plant. The locus relevant to this method most commonly comprises the growth medium (i.e. medium providing nutrients to the plant), typically soil in which the plant is grown. Treatment of a crop plant to increase vigor of the crop plant thus comprises contacting the crop plant, the seed from which the crop plant is grown or the locus of the crop plant with a biologically effective amount of a compound of Formula 1.

Increased crop vigor can result in one or more of the following observed effects: (a) optimal crop establishment as demonstrated by excellent seed germination, crop emergence and crop stand; (b) enhanced crop growth as demonstrated by rapid and robust leaf growth (e.g., measured by leaf area index), plant height, number of tillers (e.g., for rice), root mass and overall dry weight of vegetative mass of the crop; (c) improved crop yields, as demonstrated by time to flowering, duration of flowering, number of flowers, total biomass accumulation (i.e. yield quantity) and/or fruit or grain grade marketability of produce (i.e. yield quality); (d) enhanced ability of the crop to withstand or prevent plant disease infections and arthropod, nematode or mollusk pest infestations; and (e) increased ability of the crop to withstand environmental stresses such as exposure to thermal extremes, suboptimal moisture or phytotoxic chemicals.

The compounds of the present invention may increase the vigor of treated plants compared to untreated plants by killing or otherwise preventing feeding of phytophagous invertebrate pests in the environment of the plants. In the absence of such control of phytophagous invertebrate pests, the pests reduce plant vigor by consuming plant tissues or sap, or transmitting plant pathogens such as viruses. Even in the absence of phytophagous invertebrate pests, the compounds of the invention may increase plant vigor by modifying metabolism of plants. Generally, the vigor of a crop plant will be most significantly increased by treating the plant with a compound of the invention if the plant is grown in a nonideal environment, i.e. an environment comprising one or more aspects adverse to the plant achieving the full genetic potential it would exhibit in an ideal environment.

Of note is a method for increasing vigor of a crop plant wherein the crop plant is grown in an environment comprising phytophagous invertebrate pests. Also of note is a method for increasing vigor of a crop plant wherein the crop plant is grown in an environment not comprising phytophagous invertebrate pests. Also of note is a method for increasing vigor of a crop plant wherein the crop plant is grown in an environment comprising an amount of moisture less than ideal for supporting growth of the crop plant. Of note is a method for increasing vigor of a crop plant wherein the crop is rice. Also of note is a method for increasing vigor of a crop plant wherein the crop is maize (corn). Also of note is a method for increasing vigor of a crop plant wherein the crop is soybean.

Compounds of this invention can also be mixed with one or more other biologically active compounds or agents including insecticides, fungicides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, other biologically active compounds or entomopathogenic bacteria, virus or fungi to form a multi-component pesticide giving an even broader spectrum of agronomic and nonagronomic utility. Thus the present invention also pertains to a composition comprising a biologically effective amount of a compound of Formula 1, at least one additional component selected from the group consisting of surfactants, solid diluents and liquid diluents, and at least one additional biologically active compound or agent. For mixtures of the present invention, the other biologically active compounds or agents can be formulated together with the present compounds, including the compounds of Formula 1, to form a premix, or the other biologically active compounds or agents can be formulated separately from the present compounds, including the compounds of Formula 1, and the two formulations combined together before application (e.g., in a spray tank) or, alternatively, applied in succession.

Examples of such biologically active compounds or agents with which compounds of this invention can be formulated are insecticides such as abamectin, acephate, acequinocyl, acetamiprid, acrinathrin, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11H-naphtho[2,1-b]pyrano[3,4-e]pyran-4-yl]methyl cyclopropanecarboxylate), amidoflumet, amitraz, avermectin, azadirachtin, azinphos-methyl, benfuracarb, bensultap, bifenthrin, bifenazate, bistrifluron, borate, buprofezin, cadusafos, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyclaniliprole (3-bromo-N-[2-bromo-4-chloro-6-[[(1-cyclopropylethyl)anino]carbonyl]phenyl]-1-(3-chloro-2-pyridinyl)-1H-pyrazole-5-carboxamiide), cycloprothrin, cycloxaprid ((5S,8R)-1-[(6-chloro-3-pyridinyl)methyl]-2,3,5,6,7,8-hexahydro-9-nitro-5,8-Epoxy-1H-imidazo[1,2-a]azepine) cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenitrothion, fenothiocarb, fenoxycarb, fenpropathrin, fenvalerate, fipronil, flometoquin (2-ethyl-3,7-dimethyl-6-[4-(trifluoromethoxy)phenoxy]-4-quinolinyl methyl carbonate), flonicamid, flubendiamide, flucythrinate, flufenerim, flufenoxuron, flufenoxystrobin (methyl (αE)-2-[[2-chloro-4-(trifluoromethyl)phenoxy]methyl]-a-(methoxymethylene)benzeneacetate), flufensulfone (5-chloro-2-[(3,4,4-trifluoro-3-buten-1-yl)sulfonyl]thiazole), fluhexafon, fluopyram, flupiprole (1-[2,6-dichloro-4-(trifluoromethyl)phenyl]-5-[(2-methyl-2-propen-1-yl)amino]-4-[(trifluoromethyl)sulfinyl]-1H-pyrazole-3-carbonitrile), flupyradifurone (4-[[(6-chloro-3-pyridinyl)methyl](2,2-difluoroethyl)amino]-2(5H)-furanone), fluvalinate, tau-fluvalinate, fonophos, formetanate, fosthiazate, halofenozide, heptafluthrin ([2,3,5,6-tetrafluoro-4-(methoxymethyl)phenyl]methyl 2,2-dimethyl-3-[(1Z)-3,3,3-trifluoro-1-propen-1-yl]cyclopropanecarboxylate), hexaflumuron, hexythiazox, hydramethylnon, imidacloprid, indoxacarb, insecticidal soaps, isofenphos, lufenuron, malathion, meperfluthrin ([2,3,5,6-tetrafluoro-4-(methoxymethyl)phenyl]methyl (1R,3S)-3-(2,2-dichloroethenyl)-2,2-dimethylcyclopropanecarboxylate), metaflumizone, metaldehyde, methamidophos, methidathion, methiodicarb, methomyl, methoprene, methoxychlor, metofluthrin, methoxyfenozide, metofluthrin, monocrotophos, monofluorothrin ([2,3,5,6-tetrafluoro-4-(methoxymethyl)phenyl]methyl 3-(2-cyano-1-propen-1-yl)-2,2-dimethylcyclopropanecarboxylate), nicotine, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), pymetrozine, pyrafluprole, pyrethrin, pyridaben, pyridalyl, pyrifluquinazon, pyriminostrobin (methyl (αE)-2-[[[2-[(2,4-dichlorophenyl)amino]-6-(trifluoromethyl)-4-pyrimidinyl]oxy]methyl]-α-(methoxymethylene)benzeneacetate), pyriprole, pyriproxyfen, rotenone, ryanodine, silafluofen, spinetoram, spinosad, spirodiclofen, spiromesifen, spirotetramat, sulprofos, sulfoxaflor (N-[methyloxido[1-[6-(trifluoromethyl)-3-pyridinyl]ethyl]-λ⁴-sulfanylidene]cyanamide), tebufenozide, tebufenpyrad, teflubenzuron, tefluthrin, terbufos, tetrachlorvinphos, tetramethrin, tetramethylfluthrin ([2,3,5,6-tetrafluoro-4-(methoxymethyl)phenyl]methyl 2,2,3,3-tetramethylcyclopropanecarboxylate), tetraniliprole, thiacloprid, thiamethoxam, thiodicarb, thiosultap-sodium, tioxazafen (3-phenyl-5-(2-thienyl)-1,2,4-oxadiazole), tolfenpyrad, tralomethrin, triazamate, trichlorfon, triflumezopyrim (2,4-dioxo-1-(5-pyrimidinylmethyl)-3-[3-(trifluoromethyl)phenyl]-2H-pyrido[1,2-a]pyrimidinium inner salt), triflumuron, Bacillus thuringiensis delta-endotoxins, entomopathogenic bacteria, entomopathogenic viruses and entomopathogenic fungi.

Of note are insecticides such as abamectin, acetamiprid, acrinathrin, afidopyropen, amitraz, avermectin, azadirachtin, benfuracarb, bensultap, bifenthrin, buprofezin, cadusafos, carbaryl, cartap, chlorantraniliprole, chlorfenapyr, chlorpyrifos, clothianidin, cyantraniliprole, cyclaniliprole, cycloprothrin, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenitrothion, fenothiocarb, fenoxycarb, fenvalerate, fipronil, flometoquin, flonicamid, flubendiamide, flufenoxuron, flufenoxystrobin, flufensulfone, flupiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiodicarb, methomyl, methoprene, methoxyfenozide, metofluthrin, monofluorothrin, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumezopyrim, triflumuron, Bacillus thuringiensis delta-endotoxins, all strains of Bacillus thuringiensis and all strains of nucleo polyhedrosis viruses.

One embodiment of biological agents for mixing with compounds of this invention include entomopathogenic bacteria such as Bacillus thuringiensis, and the encapsulated delta-endotoxins of Bacillus thuringiensis such as MVP® and MVPII® bioinsecticides prepared by the CellCap® process (CellCap®, MVP® and MVPII® are trademarks of Mycogen Corporation, Indianapolis, Indiana, USA); entomopathogenic fungi such as green muscardine fungus; and entomopathogenic (both naturally occurring and genetically modified) viruses including baculovirus, nucleopolyhedro virus (NPV) such as Helicoverpa zea nucleopolyhedrovirus (HzNPV), Anagrapha falcifera nucleopolyhedrovirus (AfNPV); and granulosis virus (GV) such as Cydia pomonella granulosis virus (CpGV).

Of particular note is such a combination where the other invertebrate pest control active ingredient belongs to a different chemical class or has a different site of action than the compound of Formula 1. In certain instances, a combination with at least one other invertebrate pest control active ingredient having a similar spectrum of control but a different site of action will be particularly advantageous for resistance management. Thus, a composition of the present invention can further comprise a biologically effective amount of at least one additional invertebrate pest control active ingredient having a similar spectrum of control but belonging to a different chemical class or having a different site of action. These additional biologically active compounds or agents include, but are not limited to, acetylcholinesterase (AChE) inhibitors such as the carbamates methomyl, oxamyl, thiodicarb, triazamate, and the organophosphates chlorpyrifos; GABA-gated chloride channel antagonists such as the cyclodienes dieldrin and endosulfan, and the phenylpyrazoles ethiprole and fipronil; sodium channel modulators such as the pyrethroids bifenthrin, cyfluthrin, beta-cyfluthrin, cyhalothrin, lambda-cyhalothrin, cypermethrin, deltamethrin, dimefluthrin, esfenvalerate, metofluthrin and profluthrin; nicotinic acetylcholinereceptor (nAChR) agonists such as the neonicotinoids acetamiprid, clothianidin, dinotefuran, imidacloprid, nitenpyram, nithiazine, thiacloprid, and thiamethoxam, and sulfoxaflor; nicotinic acetylcholine receptor (nAChR) allosteric activators such as the spinosyns spinetoram and spinosad; chloride channel activators such as the avermectins abamectin and emamectin; juvenile hormone mimics such as diofenolan, methoprene, fenoxycarb and pyriproxyfen; selective homopteran feeding blockers such as pymetrozine and flonicamid; mite growth inhibitors such as etoxazole; inhibitors of mitochondrial ATP synthase such as propargite; ucouplers of oxidative phosphorylation via disruption of the proton gradient such as chlorfenapyr; nicotinic acetylcholine receptor (nAChR) channel blockers such as the nereistoxin analogs cartap; inhibitors of chitin biosynthesis such as the benzoylureas flufenoxuron, hexaflumuron, lufenuron, novaluron, noviflumuron and triflumuron, and buprofezin; dipteran moulting disrupters such as cyromazine; ecdysone receptor agonists such as the diacylhydrazines methoxyfenozide and tebufenozide; octopamine receptor agonists such as amitraz; mitochondrial complex III electron transport inhibitors such as hydramethylnon; mitochondrial complex I electron transport inhibitors such as pyridaben; voltage-dependent sodium channel blockers such as indoxacarb; inhibitors of acetyl CoA carboxylase such as the tetronic and tetramic acids spirodiclofen, spiromesifen and spirotetramat; mitochondrial complex II electron transport inhibitors such as the B-ketonitriles cyenopyrafen and cyflumetofen; ryanidine receptor modulators such as the anthranilic diamides chlorantraniliprole, cyantraniliprole and cyantraniliprole, diamides such as flubendiamide, and ryanodine receptor ligands such as ryanodine; compounds wherein the target site responsible for biological activity is unknown or uncharacterized such as azadirachtin, bifenazate, pyridalyl, pyrifluquinazon and triflumezopyrim; microbial disrupters of insect midgut membranes such as Bacillus thuringensis and the delta-endotoxins they produce and Bacillus sphaericus; and biological agents including nucleo polyhedro viruses (NPV) and other naturally occurring or genetically modified insecticidal viruses.

Further examples of biologically active compounds or agents with which compounds of this invention can be formulated are: fungicides such as acibenzolar-S-methyl, aldimorph, ametoctradin, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bitertanol, bixafen, blasticidin-S, boscalid, bromuconazole, bupirimate, buthiobate, carboxin, carpropamid, captafol, captan, carbendazim, chloroneb, chlorothalonil, chlozolinate, copper hydroxide, copper oxychloride, copper sulfate, coumoxystrobin, cyazofamid, cyflufenamid, cymoxanil, cyproconazole, cyprodinil, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dithianon, dithiolanes, dodemorph, dodine, econazole, etaconazole, edifenphos, enoxastrobin (also known as enestroburin), epoxiconazole, ethaboxam, ethirimol, etridiazole, famoxadone, fenamidone, fenaminstrobin, fenarimol, fenbuconazole, fenfuram, fenhexamide, fenoxanil, fenpiclonil, fenpropidin, fenpropimorph, fenpyrazamine, fentin acetate, fentin hydroxide, ferbam, ferimzone, flometoquin, fluazinam, fludioxonil, flufenoxystrobin, flumorph, fluopicolide, fluopyram, fluoxastrobin, fluquinconazole, flusilazole, flusulfamide, flutianil, flutolanil, flutriafol, fluxapyroxad, folpet, fthalide (also known as phthalide), fuberidazole, furalaxyl, furametpyr, hexaconazole, hymexazole, guazatine, imazalil, imibenconazole, iminoctadine albesilate, iminoctadine triacetate, iodicarb, ipconazole, isofetamid, iprobenfos, iprodione, iprovalicarb, isoprothiolane, isopyrazam, isotianil, kasugamycin, kresoxim-methyl, mancozeb, mandipropamid, mandestrobin, maneb, mapanipyrin, mepronil, meptyldinocap, metalaxyl (including metalaxyl-M/mefenoxam), metconazole, methasulfocarb, metiram, metominostrobin, metrafenone, myclobutanil, naftitine, neo-asozin (ferric methanearsonate), nuarimol, octhilinone, ofurace, orysastrobin, oxadixyl, oxathiapiprolin, oxolinic acid, oxpoconazole, oxycarboxin, oxytetracycline, penconazole, pencycuron, penflufen, penthiopyrad, perfurazoate, phosphorous acid (including salts thereof, e.g., fosetyl-aluminm), picoxystrobin, piperalin, polyoxin, probenazole, prochloraz, procymidone, propamocarb, propiconazole, propineb, proquinazid, prothiocarb, prothioconazole, pyraclostrobin, pyrametostrobin, pyraoxystrobin, pyrazophos, pyribencarb, pyributacarb, pyrifenox, pyriofenone, perisoxazole, pyrimethanil, pyrifenox, pyrrolnitrin, pyroquilon, quinconazole, quinmethionate, quinoxyfen, quintozene, silthiofam, sedaxane, simeconazole, spiroxamine, streptomycin, sulfur, tebuconazole, tebufloquin, teclofthalam, tecloftalam, teenazene, terbinafine, tetraconazole, thiabendazole, thifluzamide, thiophanate, thiophanate-methyl, thiram, tiadinil, tolclofos-methyl, tolprocarb, tolyfluanid, triadimefon, triadimenol, triarimol, triazoxide, tribasic copper sulfate, triclopyricarb, tridemorph, trifloxystrobin, triflumizole, trimoprhamide tricyclazole, trifloxystrobin, triforine, triticonazole, uniconazole, validamycin, valifenalate (also known as valifenal), vinclozolin, zineb, ziram, zoxamide and 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone; nematocides such as fluopyram, spirotetramat, thiodicarb, fosthiazate, abamectin, iprodione, fluensulfone, dimethyl disulfide, tioxazafen, 1,3-dichloropropene (1,3-D), metam (sodium and potassium), dazomet, chloropicrin, fenamiphos, ethoprophos, cadusaphos, terbufos, imicyafos, oxamyl, carbofuran, tioxazafen, Bacillus firmus and Pasteuria nishizawae; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad.

In certain instances, combinations of a compound of this invention with other biologically active (particularly invertebrate pest control) compounds or agents (i.e. active ingredients) can result in an enhanced effect. Reducing the quantity of active ingredients released in the environment while ensuring effective pest control is always desirable. When enhanced invertebrate pest control occurs at application rates giving agronomically satisfactory levels of invertebrate pest control, such combinations can be advantageous for reducing crop production cost and decreasing environmental load.

Compounds of this invention and compositions thereof can be applied to plants genetically transformed to express proteins toxic to invertebrate pests (such as Bacillus thuringiensis delta-endotoxins). Such an application may provide a broader spectrum of plant protection and be advantageous for resistance management. The exogenously applied invertebrate pest control compounds of this invention in combination with the expressed toxin proteins may provide an enhanced effect.

General references for these agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U. K., 2003 and The BioPesticide Manual, 2^{nd} Edition, L. G. Copping, Ed., British Crop Protection Council, Farnham, Surrey, U. K., 2001.

Invertebrate pests are controlled in agronomic and nonagronomic applications by applying one or more compounds of this invention, typically in the form of a composition, in a biologically effective amount, to the environment of the pests, including the agronomic and/or nonagronomic locus of infestation, to the area to be protected, or directly on the pests to be controlled.

Thus the present invention comprises a method for controlling and combating an invertebrate pest in agronomic and/or nonagronomic applications, comprising contacting the invertebrate pest or its environment with a biologically effective amount of one or more of the compounds of the invention, or with a composition comprising at least one such compound or a composition comprising at least one such compound and a biologically effective amount of at least one additional biologically active compound or agent. Examples of suitable compositions comprising a compound of the invention and a biologically effective amount of at least one additional biologically active compound or agent include granular compositions wherein the additional active compound is present on the same granule as the compound of the invention or on granules separate from those of the compound of the invention.

To achieve contact with a compound or composition of the invention to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a compound of the invention can be applied to the plant foliage or the soil. Compounds of this invention can also be effectively delivered through plant uptake by contacting the plant with a composition comprising a compound of this invention applied as a soil drench of a liquid formulation, a granular formulation to the soil, a nursery box treatment or a dip of transplants. Of note is a composition of the present invention in the form of a soil drench liquid formulation. Also of note is a method for controlling and combating an invertebrate pest comprising contacting the invertebrate pest or its environment with a biologically effective amount of a compound of the present invention or with a composition comprising a biologically effective amount of a compound of the present invention. Of further note is this method wherein the environment is soil and the composition is applied to the soil as a soil drench formulation. Of further note is that compounds of this invention are also effective by localized application to the locus of infestation. Other methods of contact include application of a compound or a composition of the invention by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. The compounds of this invention can also be impregnated into materials for fabricating invertebrate control devices (e.g., insect netting).

Compounds of the invention are useful in treating all plants, plant parts and seeds. Plant and seed varieties and cultivars can be obtained by conventional propagation and breeding methods or by genetic engineering methods. Genetically modified plants or seeds (transgenic plants or seeds) are those in which a heterologous gene (transgene) has been stably integrated into the plant's or seed's genome. A transgene that is defined by its particular location in the plant genome is called a transformation or transgenic event.

Genetically modified plant and seed cultivars which can be treated according to the invention include those that are resistant against one or more biotic stresses (pests such as nematodes, insects, mites, fungi, etc.) or abiotic stresses (drought, cold temperature, soil salinity, etc.), or that contain other desirable characteristics. Plants and seeds can be genetically modified to exhibit traits of, for example, herbicide tolerance, insect-resistance, modified oil profiles or drought tolerance.

Treatment of genetically modified plants and seeds with compounds of the invention may result in super-additive or enhanced effects. For example, reduction in application rates, broadening of the activity spectrum, increased tolerance to biotic/abiotic stresses or enhanced storage stability may be greater than expected from just simple additive effects of the application of compounds of the invention on genetically modified plants and seeds.

Compounds of this invention are also useful in seed treatments for protecting seeds from invertebrate pests. In the context of the present disclosure and claims, treating a seed means contacting the seed with a biologically effective amount of a compound of this invention, which is typically formulated as a composition of the invention. This seed treatment protects the seed from invertebrate soil pests and generally can also protect roots and other plant parts in contact with the soil of the seedling developing from the germinating seed. The seed treatment may also provide protection of foliage by translocation of the compound of this invention or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this invention can also increase vigor of plants growing from the seed.

One method of seed treatment is by spraying or dusting the seed with a compound of the invention (i.e. as a formulated composition) before sowing the seeds. Compositions formulated for seed treatment generally comprise a film former or adhesive agent. Therefore typically a seed coating composition of the present invention comprises a biologically effective amount of a compound of Formula 1, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

Compounds of Formula 1 and their compositions, both alone and in combination with other insecticides, nematicides, and fungicides, are particularly useful in seed treatment for crops including, but not limited to, maize or corn, soybeans, cotton, cereal (e.g., wheat, oats, barley, rye and rice), potatoes, vegetables and oilseed rape.

Other insecticides with which compounds of Formula 1 can be formulated to provide mixtures useful in seed treatment include abamectin, acetamiprid, acrinathrin, amitraz, avermectin, azadirachtin, bensultap, bifenthrin, buprofezin, cadusafos, carbaryl, carbofuran, cartap, chlorantraniliprole, chlorfenapyr, chlorpyrifos, clothianidin, cyantraniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, flufenoxuron, fluvalinate, formetanate, fosthiazate, hexaflumuron, hydramethylnon, imidacloprid, indoxacarb, lufenuron, metaflumizone, methiocarb, methomyl, methoprene, methoxyfenozide, nitenpyram, nithiazine, novaluron, oxamyl, pymetrozine, pyrethrin, pyridaben, pyridalyl, pyriproxyfen, ryanodine, spinetoram, spinosad, spirodiclofen, spiromesifen, spirotetramat, sulfoxaflor, tebufenozide, tetramethrin, thiacloprid, thiamethoxam, thiodicarb, thiosultap-sodium, tralomethrin, triazamate, triflumuron, Bacillus thuringiensis delta-endotoxins, all strains of Bacillus thuringiensis and all strains of nucleo polyhedrosis viruses.

Fungicides with which compounds of Formula 1 can be formulated to provide mixtures useful in seed treatment include amisulbrom, azoxystrobin, boscalid, carbendazim, carboxin, cymoxanil, cyproconazole, difenoconazole, dimethomorph, fluazinam, fludioxonil, fluquinconazole, fluopicolide, fluoxastrobin, flutriafol, fluxapyroxad, ipconazole, iprodione, metalaxyl, mefenoxam, metconazole, myclobutanil, paclobutrazole, penflufen, picoxystrobin, prothioconazole, pyraclostrobin, sedaxane, silthiofam, tebuconazole, thiabendazole, thiophanate-methyl, thiram, trifloxystrobin and triticonazole.

Compositions comprising compounds of Formula 1 useful for seed treatment can further comprise bacteria and fungi that have the ability to provide protection from the harmful effects of plant pathogenic fungi or bacteria and/or soil born animals such as nematodes. Bacteria exhibiting nematicidal properties may include but are not limited to Bacillus firmus, Bacillus cereus, Bacillius subtiliis and Pasteuria penetrans. A suitable Bacillus firmus strain is strain CNCM I-1582 (GB-126) which is commercially available as BioNem™. A suitable Bacillus cereus strain is strain NCMM I-1592. Both Bacillus strains are disclosed in U.S. Pat. No. 6,406,690. Other suitable bacteria exhibiting nematicidal activity are B. amyloliquefaciens IN937a and B. subtilis strain GB03. Bacteria exhibiting fungicidal properties may include but are not limited to B. pumilus strain GB34. Fungal species exhibiting nematicidal properties may include but are not limited to Myrothecium verrucaria, Paecilomyces lilacinus and Purpureocillium lilacinum.

Seed treatments can also include one or more nematicidal agents of natural origin such as the elicitor protein called harpin which is isolated from certain bacterial plant pathogens such as Erwinia amylovora. An example is the Harpin-N-Tek seed treatment technology available as N-Hibit™ Gold CST.

Seed treatments can also include one or more species of legume-root nodulating bacteria such as the microsymbiotic nitrogen-fixing bacteria Bradyrhizobium japonicum. These inocculants can optionally include one or more lipo-chitooligosaccharides (LCOs), which are nodulation (Nod) factors produced by rhizobia bacteria during the initiation of nodule formation on the roots of legumes. For example, the Optimize® brand seed treatment technology incorporates LCO Promoter Technology™ in combination with an inocculant.

Seed treatments can also include one or more isoflavones which can increase the level of root colonization by mycorrhizal fungi. Mycorrhizal fungi improve plant growth by enhancing the root uptake of nutrients such as water, sulfates, nitrates, phosphates and metals. Examples of isoflavones include, but are not limited to, genistein, biochanin

A, formononetin, daidzein, glycitein, hesperetin, naringenin and pratensein. Formononetin is available as an active ingredient in mycorrhizal inocculant products such as PHC Colonize® AG.

Seed treatments can also include one or more plant activators that induce systemic acquired resistance in plants following contact by a pathogen. An example of a plant activator which induces such protective mechanisms is acibenzolar-S-methyl.

The treated seed typically comprises a compound of the present invention in an amount from about 0.1 g to 1 kg per 100 kg of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this invention can be incorporated into a bait composition that is consumed by an invertebrate pest or used within a device such as a trap, bait station, and the like. Such a bait composition can be in the form of granules which comprise (a) active ingredients, namely a biologically effective amount of a compound of Formula 1; (b) one or more food materials; optionally (c) an attractant, and optionally (d) one or more humectants. Of note are granules or bait compositions which comprise between about 0.001-5% active ingredients, about 40-99% food material and/or attractant; and optionally about 0.05-10% humectants, which are effective in controlling and combating soil invertebrate pests at very low application rates, particularly at doses of active ingredient that are lethal by ingestion rather than by direct contact. Some food materials can function both as a food source and an attractant. Food materials include carbohydrates, proteins and lipids. Examples of food materials are vegetable flour, sugar, starches, animal fat, vegetable oil, yeast extracts and milk solids. Examples of attractants are odorants and flavorants, such as fruit or plant extracts, perfume, or other animal or plant component, pheromones or other agents known to attract a target invertebrate pest. Examples of humectants, i.e. moisture retaining agents, are glycols and other polyols, glycerine and sorbitol. Of note is a bait composition (and a method utilizing such a bait composition) used to control at least one invertebrate pest selected from the group consisting of ants, termites and cockroaches. A device for controlling and combating an invertebrate pest can comprise the present bait composition and a housing adapted to receive the bait composition, wherein the housing has at least one opening sized to permit the invertebrate pest to pass through the opening so the invertebrate pest can gain access to the bait composition from a location outside the housing, and wherein the housing is further adapted to be placed in or near a locus of potential or known activity for the invertebrate pest.

One embodiment of the present invention relates to a method for controlling and combating invertebrate pests, comprising diluting the pesticidal composition of the present invention (a compound of Formula 1 formulated with surfactants, solid diluents and liquid diluents or a formulated mixture of a compound of Formula 1 and at least one other pesticide) with water, and optionally adding an adjuvant to form a diluted composition, and contacting the invertebrate pest or its environment with an effective amount of said diluted composition.

Although a spray composition formed by diluting with water a sufficient concentration of the present pesticidal composition can provide sufficient efficacy for controlling and combating invertebrate pests, separately formulated adjuvant products can also be added to spray tank mixtures. These additional adjuvants are commonly known as “spray adjuvants” or “tank-mix adjuvants”, and include any substance mixed in a spray tank to improve the performance of a pesticide or alter the physical properties of the spray mixture. Adjuvants can be surfactants, emulsifying agents, petroleum-based crop oils, crop-derived seed oils, acidifiers, buffers, thickeners or defoaming agents. Adjuvants are used to enhancing efficacy (e.g., biological availability, adhesion, penetration, uniformity of coverage and durability of protection), or minimizing or eliminating spray application problems associated with incompatibility, foaming, drift, evaporation, volatilization and degradation. To obtain optimal performance, adjuvants are selected with regard to the properties of the active ingredient, formulation and target (e.g., crops, insect pests).

Among the spray adjuvants, oils including crop oils, crop oil concentrates, vegetable oil concentrates and methylated seed oil concentrates are most commonly used to improve the efficacy of pesticides, possibly by means of promoting more even and uniform spray deposits. In situations where phytotoxicity potentially caused by oils or other water-immiscible liquids are of concern, spray compositions prepared from the composition of the present invention will generally not contain oil-based spray adjuvants. However, in situations where phytotoxicity caused by oil-based spray adjuvants is commercially insignificant, spray compositions prepared from the composition of the present composition can also contain oil-based spray adjuvants, which can potentially further increase control of invertebrate pests, as well as rainfastness.

Products identified as “crop oil” typically contain 95 to 98% paraffin or naphtha-based petroleum oil and 1 to 2% of one or more surfactants functioning as emulsifiers. Products identified as “crop oil concentrates” typically consist of 80 to 85% of emulsifiable petroleum-based oil and 15 to 20% of nonionic surfactants. Products correctly identified as “vegetable oil concentrates” typically consist of 80 to 85% of vegetable oil (i.e. seed or fruit oil, most commonly from cotton, linseed, soybean or sunflower) and 15 to 20% of nonionic surfactants. Adjuvant performance can be improved by replacing the vegetable oil with methyl esters of fatty acids that are typically derived from vegetable oils. Examples of methylated seed oil concentrates include MSO® Concentrate (UAP-Loveland Products, Inc.) and Premium MSO Methylated Spray Oil (Helena Chemical Company).

The amount of adjuvants added to spray mixtures generally does not exceed about 2.5% by volume, and more typically the amount is from about 0.1 to about 1% by volume. The application rates of adjuvants added to spray mixtures are typically between about 1 to 5 L per hectare. Representative examples of spray adjuvants include: Adigor® (Syngenta) 47% methylated rapeseed oil in liquid hydrocarbons, Silwet® (Helena Chemical Company) polyalkyleneoxide modified heptamethyltrisiloxane and Assist® (BASF) 17% surfactant blend in 83% paraffin based mineral oil.

The compounds of this invention can be applied without other adjuvants, but most often application will be of a formulation comprising one or more active ingredients with suitable carriers, diluents, and surfactants and possibly in combination with a food depending on the contemplated end use. One method of application involves spraying a water dispersion or refined oil solution of a compound of the present invention. Combinations with spray oils, spray oil concentrations, spreader stickers, adjuvants, other solvents, and piperonyl butoxide often enhance compound efficacy. For nonagronomic uses such sprays can be applied from spray containers such as a can, a bottle or other container, either by means of a pump or by releasing it from a pressurized container, e.g., a pressurized aerosol spray can. Such spray compositions can take various forms, for example, sprays, mists, foams, fumes or fog. Such spray compositions thus can further comprise propellants, foaming agents, etc. as the case may be. Of note is a spray composition comprising a biologically effective amount of a compound or a composition of the present invention and a carrier. One embodiment of such a spray composition comprises a biologically effective amount of a compound or a composition of the present invention and a propellant. Representative propellants include, but are not limited to, methane, ethane, propane, butane, isobutane, butene, pentane, isopentane, neopentane, pentene, hydrofluorocarbons, chlorofluorocarbons, dimethyl ether, and mixtures of the foregoing. Of note is a spray composition (and a method utilizing such a spray composition dispensed from a spray container) used to control at least one invertebrate pest selected from the group consisting of mosquitoes, black flies, stable flies, deer flies, horse flies, wasps, yellow jackets, hornets, ticks, spiders, ants, gnats, and the like, including individually or in combinations.

The following Tests demonstrate the control efficacy of compounds of this invention on specific pests. “Control efficacy” represents inhibition of invertebrate pest development (including mortality) that causes significantly reduced feeding. The pest control protection afforded by the compounds is not limited, however, to these species. See Index Table A for compound descriptions.

### BIOLOGICAL EXAMPLES

The following Tests demonstrate the control efficacy of compounds of this disclosure on specific pests. “Control efficacy” represents inhibition of invertebrate pest development (including mortality) that causes significantly reduced feeding. The pest control protection afforded by the compounds is not limited, however, to these species. See Index Table A for compound descriptions.

### Formulation and Spray Methodology for Tests A-H

Test compounds were formulated using a solution containing 10% acetone, 90% water and 300 ppm Activator 90@non-ionic surfactant (Loveland Products, Loveland, Colorado, USA). The formulated compounds were applied in 1 mL of liquid through an atomized nozzle positioned 1.27 cm (0.5 inches) above the top of each test unit. Test compounds were sprayed at the rates indicated, and each test was replicated three times.

### Test A

For evaluating control of Diamond Back Moth (Plutella xylostella L.) through contact and/or systemic means, each test unit consisted of a small open container with a 10- to 12-day-old mustard plant inside.

Test compounds were formulated and sprayed at 250, and 50 ppm with three replications as described above. After spraying, the test units were allowed to dry for 1 hour before they were infested with 30-50 neonate larvae. A black, screened cap was placed on the top of each container. The test units were held for six days in a growth chamber at 24-25° C. and 70% relative humidity. Plant feeding damage was then visually assessed based on foliage consumed, and larvae were assessed for mortality.

Of the compounds of Formula 1 tested at 250 ppm, the following provided very good to excellent levels of control efficacy (20% or less feeding damage): 1, 2, 3, 5, 9, 12, 13, 14, 41, 43, 46, 48, 52, 55, 96, 104, 105, 109, 112, 113, 114, 116, 119, 125.

Of the compounds of Formula 1 tested at 50 ppm, the following provided very good to excellent levels of control efficacy (20% or less feeding damage): 9, 12, 14, 41, 46, 50, 52, 61, 77, 79, 80, 96, 104, 105, 109, 112, 113, 114, 125.

Of the compounds of Formula 1 tested at 10 ppm, the following provided very good to excellent levels of control efficacy (20% or less feeding damage): 12, 14, 41, 50, 61, 79, 80, 96.

Of the compounds of Formula 1 tested at 2 ppm, the following provided very good to excellent levels of control efficacy (20% or less feeding damage): 12, 14.

### Test B

For evaluating control of fall armyworm (Spodoptera frugiperda (J. E. Smith)) the test unit consisted of a small open container with a 4- to 5-day-old corn (maize) plant inside. This was pre-infested with 10 to 15 one-day-old larvae on a piece of insect diet.

Test compounds were formulated and sprayed at 250, 50, 10, and 2 ppm with three replications as described above. After spraying of the formulated test compound, the test units were maintained in a growth chamber for 6 days at 25° C. and 70% relative humidity. Plant feeding damage was then visually assessed based on foliage consumed, and larvae were assessed for mortality.

Of the compounds of Formula 1 tested at 250 ppm, the following provided very good to excellent levels of control efficacy (20% or less feeding damage): 12, 14, 46, 52, 70, 96, 109, 114, 125.

Of the compounds of Formula 1 tested at 50 ppm, the following provided very good to excellent levels of control efficacy (20% or less feeding damage): 12, 14, 41, 46, 50, 52, 61, 112, 114, 125.

Of the compounds of Formula 1 tested at 10 ppm, the following provided very good to excellent levels of control efficacy (20% or less feeding damage): 12, 14, 41.

### Test C

For evaluating control of cotton melon aphid (Aphis gossypii (Glover)) through contact and/or systemic means, the test unit consisted of a small open container with a 5-day-old okra plant inside. This was pre-infested with 30-40 insects on a piece of leaf according to the cut-leaf method, and the soil of the test unit was covered with a layer of sand.

Test compounds were formulated and sprayed at 250, 50, 10, and 2 ppm with three replications as described above. After spraying, the test units were maintained in a growth chamber for 6 days at 19° C. and 70% relative humidity. Each test unit was then visually assessed for insect mortality.

Of the compounds of Formula 1 tested at 250 ppm, the following resulted in at least 80% mortality: 96.

What is claimed is:

1. A compound of Formula 1 (including all geometric and stereoisomers), N-oxides, and salts thereof: 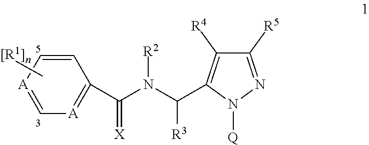
wherein
X is O or S;
each A is independently CH, N or CR¹;
each R¹ is independently H, amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or
R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸;
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or R² is C(O)R⁷, C(O)OR⁷, NR⁷R⁸, OR⁷, S(O)ₚR⁹ or SO₂NR⁷R⁸;
R³ is C₁-C₃ alkyl or C₁-C₃ haloalkyl;
R⁴ is hydrogen, cyano, halogen, nitro, C(S)NH₂, SCN, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R⁴ is C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸;
R⁵ is hydrogen, cyano, halogen, nitro, C(S)NH₂, SCN, C₁-C₆-alkyl, C₂-C₆ alkenyl, C₂-C₆ alkynyl C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R⁵ is C(O)R⁷, CR⁷(═NO)R⁸, C(O)OR⁷, C(O)NR⁷R⁸, NR⁷R⁸, NR⁸C(O)R⁷, NR⁸C(O)OR⁷, NR¹¹SO₂NR⁷R⁸, OR⁷, S(O)ₚR⁹, or SO₂NR⁷R⁸;
each R⁶ is independently halogen, cyano, nitro, SF₅, C(O)NH₂, C(S)NH₂, C₁-C₃ alkyl, C₁-C₃ haloalkyl, C₁-C₃ alkoxy, C₁-C₃ haloalkoxy, C₃-C₇ cycloalkyl, C₃-C₉ trialkylsilyl, C(O)OR¹⁰, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)OR⁷, S(O)ₚR⁹, SO₂NR⁷R⁸, or S(═NR)OₚR⁷;
each R⁷ is independently hydrogen, C₁-C₆ alkyl, C₂-C₆ alkenyl, C₃-C₆ alkynyl, or C₃-C₇ cycloalkyl, or C₄-C₈ cycloalkylalkyl, wherein each alkyl, alkenyl, alkynyl or cycloalkyl or cycloalkylalkyl is optionally substituted with one or more R¹²; or R⁷ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R¹²;
each R⁸ is independently hydrogen, C₁-C₆ alkyl, C₁-C₆ alkoxy, C₂-C₆ alkenyl, C₃-C₆ alkenyloxy, C₂-C₆ alkynyl, or C₃-C₆ alkynyloxy, wherein each alkyl, alkoxy, alkenyl, alkenyloxy, alkynyl, or alkynyloxy is optionally substituted with one or more R⁶;
when R⁷ and R⁸ are on N, R⁷ and R⁸ can form a ring of 3 to 7 carbons, optionally including oxygen, S(O)p or NR⁷;
each R⁹ is independently C₁-C₆ alkyl, C₂-C₆ alkenyl, C₂-C₆ alkynyl, or C₃-C₇ cycloalkyl, wherein each alkyl, alkenyl, alkynyl or cycloalkyl is optionally substituted with one or more R⁶;
or R⁹ is phenyl, or a 4 to 6-membered saturated, partly saturated, or fully unsaturated heterocyclic ring containing ring members selected from carbon atoms and 1 to 3 heteroatoms independently selected from 1 oxygen, 1 sulfur, and up to 3 nitrogen atoms, wherein each phenyl or 4- to 6-membered heterocyclic ring is optionally substituted with one or more R⁶;
each R¹⁰ is independently H, C₁-C₆ alkyl, C₁-C₆ haloalkyl, C₃-C₇ cycloalkyl, C₃-C₇ cycloalkyl-C₁-C₃-alkyl, C₁-C₃ alkoxy-C₁-C₃-alkyl;
each R₁₁ is independently hydrogen, C₁-C₆ alkyl, C₂-C₆ alkenyl, C₂-C₆ alkynyl, C₃-C₆ cycloalkyl, or C₄-C₇ cycloalkylalkyl wherein each alkyl, alkenyl, alkynyl, cycloalkyl or cycloalkylalkyl is optionally substituted with one or more R⁶; C(O)R⁷, C(O)OR⁷, or S(O)ₚR⁷;
R¹² is halogen, hydroxy, cyano, nitro, SF₅, C(O)NH₂, C(S)NH₂, C₁-C₃ alkyl, C₁-C₃ alkoxy, C₂-C₆ alkenyl, C₂-C₆ alkynyl, C₃-C₇ cycloalkyl, C₄-C₈ cycloalkylalkyl, wherein each alkyl, alkoxy, alkenyl, alkynyl, cycloalkyl or cycloalkylalkyl is optionally substituted with halogen or cyano;
Q is pyridine, pyridazine, pyrimidine, pyrazine, or thiazole wherein the pyridine, pyridazine, pyrimidine, or pyrazine is optionally substituted with one to three groups, and wherein thiazole is optionally substituted with one to two groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸;
n is 1, 2, or 3;
p is 0, 1, or 2.

2. The compound of claim 1 wherein X is O.

3. The compound of claim 1 wherein X is S.

4. The compound of claim 1 wherein A is CH.

5. The compound of any one of the foregoing claims wherein wherein A is N or CR¹.

6. The compound of claim 1 wherein
X is O;
A is CH; and
R³ is Me.

7. The compound of any one of the foregoing claims wherein R¹ is amino, cyano, halogen, nitro, C(S)NH₂; or R¹ is C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R¹ is phenyl, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heteroaromatic ring is optionally substituted with one or more R⁶; or R¹ is C₃-C₉-trialkylsilyl, C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, NHC(O)NR⁷R⁸, NR⁷S(O)ₚR⁹, OC(O)R⁷, OC(O)NR⁷R⁸, OC(O)OR⁷, S(O)ₚR⁹, SF₅, SO₂NR⁷R⁸, OS(O)₂R⁹, or S(═NR¹¹)OₚR⁹; or R¹ is a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, or SO₂NR⁷R⁸.

8. The compound of any one of the foregoing claims wherein
R² is hydrogen, C₁-C₆ alkyl, C₂-C₆ alkenyl, C₂-C₆ alkynyl, C₃-C₆ cycloalkyl, or C₄-C₇ cycloalkylalkyl, wherein each alkyl, alkenyl, alkynyl, cycloalkyl or cycloalkylalkyl is optionally substituted with one or more R⁶; or R² is C₁-C₆ alkyl optionally substituted with one or more R⁶ and substituted by a phenyl or a 4 to 6-membered saturated or partially saturated heterocyclic ring containing ring members selected from carbon atoms and from 1 to 3 heteroatoms independently selected from 1 oxygen, 1 sulfur, and up to 2 nitrogen atoms, or a 5-membered heteroaromatic ring containing ring members selected from carbon atoms and from 1 to 3 heteroatoms independently selected from 1 oxygen, 1 sulfur, and up to 3 nitrogen atoms, or a 6-membered heteroaromatic ring containing ring members selected from carbon atoms and 1 to 2 nitrogen atoms, wherein each phenyl or heterocyclic ring is optionally substituted with one or more R⁶; or
R² is C(O)R⁷, C(O)OR⁷, NR⁷R⁸, OR⁷, S(O)ₚR⁹ or SO₂NR⁷R⁸.

9. The compound of claim 7 wherein R² is H, CH₂c-Pr, or Me.

10. The compound of any one of the foregoing claims wherein
R⁴ is hydrogen, cyano, halogen, nitro, C(S)NH₂, SCN, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R⁴ is C(O)R⁷, CR⁷(═NO)R⁷, C(O)OR⁷, C(O)NR⁷R⁸, NR⁷R⁸, NR⁸C(O)R⁷, NR⁸C(O)OR⁷, OR⁷, S(O)ₚR⁹, SO₂NR⁷R⁸, or NR¹¹SO₂NR⁷R⁸; and
R⁵ is hydrogen, cyano, halogen, nitro, C(S)NH₂, SCN, C₁-C₆-alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶; or R⁵ is C(O)R⁷, CR⁷(═NO)R⁸, C(O)OR⁷, C(O)NR⁷R⁸, NR⁷R⁸, NR⁸C(O)R⁷, NR⁸C(O)OR⁷, NR¹¹SO₂NR⁷R⁸, OR⁷, S(O)ₚR⁹, or SO₂NR⁷R⁸.

11. The compound of any one of the foregoing claims wherein
Q is pyridine, or pyrimidine optionally substituted with one to three groups, these groups being chosen from amino, cyano, halogen, nitro, C₁-C₆ alkyl, C₂-C₆ alkenyl, C₂-C₆ alkynyl, C₃-C₇ cycloalkyl, or C₄-C₈ cycloalkylalkyl, wherein each alkyl, alkenyl, alkynyl, cycloalkyl or cycloalkylalkyl is optionally substituted with one or more R⁶, C(O)R⁷, CR⁷(═NOR⁸), C(O)OR⁷, C(O)NR⁷R⁸, C(S)NR⁷R⁸, OR⁷, S(O)ₚR⁹, SO₂NR⁷R⁸; NR⁷R⁸, NR⁸C(O)R⁷, NR⁸C(O)OR⁷, S(O)ₚR⁷, SO₂NR⁷R⁸, or a 5-membered or 6-membered heteroaromatic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or a 3-membered to 7-membered partly or fully saturated heterocyclic ring containing ring members selected from carbon atoms and 1-4 heteroatoms independently selected from 1 oxygen, 1 sulfur, and 1-4 nitrogen atoms optionally substituted by 1 to 3 substituents independently selected from halogen, cyano, C₁-C₃ alkyl, C₁-C₃-haloalkyl, C₁-C₃-alkoxy, and C₁-C₃-haloalkoxy, or when a nitrogen atom is present, substituted by C(O)R⁷, C(O)OR⁷, C(O)NR⁷R⁸, S(O)ₚR⁷, SO₂NR⁷R⁸.

12. The compound of any one of the foregoing claims wherein
Q is selected from Q-1, Q-2, Q-6, Q-7, Q-11, Q-13, Q-14, Q-18, Q-19, Q-20, Q-21, Q-22, Q-23, Q-24, and Q-25.

13. The compound of claim 1, wherein
X is O;
A is CH;
n is 2;
each R¹ is independently Cl, F, or Br, or R¹ is Me optionally substituted with one or more R⁶; or
R¹ is OMe, SF₃, or OS(O)₂R⁹.
R² is hydrogen, C₁-C₄ alkyl, or C₄-C₆ cycloalkylalkyl, wherein each alkyl or cycloalkylalkyl is optionally substituted with one R⁶;
R⁴ is hydrogen, cyano, halogen, nitro, or C₁-C₃ alkyl wherein the alkyl is optionally substituted with one or more R⁶, or R⁴ is NR⁷R⁸, NR⁸C(O)R⁷, OR⁷, or S(O)ₚR⁹; and
R⁵ is hydrogen, cyano, halogen, nitro, or C₁-C₃-alkyl wherein each alkyl or cycloalkyl is optionally substituted with one or more R⁶; or R⁵ is OR⁷, NR⁷R⁸, NR⁸C(O)R⁷, NR⁸C(O)OR⁷, S(O)ₚR⁷, or SO₂NR⁷R⁸.

14. The compound of claim 13, wherein R⁴ and R⁵ are not both hydrogen.

15. The compound of claim 1, wherein
X is O;
A is N or CR¹;
R¹ is Cl, F, or Br, or R¹ is Me optionally substituted with one or more R⁶; or R¹ is OMe, SF₃, or OS(O)₂R⁹.
R² is hydrogen, C₁-C₄ alkyl, or C₄-C₆ cycloalkylalkyl, wherein each alkyl or cycloalkylalkyl is optionally substituted with one R⁶; or
R² is C(O)R⁷ or C(O)OR⁷; and
R⁴ is hydrogen, cyano, halogen, nitro, or C₁-C₃ alkyl wherein the alkyl is optionally substituted with one or more R⁶.

16. The compound of claim 1 wherein the compound is selected from the group consisting of
N-[1-[3-amino-4-cyano-1-(5-cyano-2pyridinyl)-1H-pyrazol-5-yl]ethyl]-N-methyl-3,5 bis(trifluoromethyl)benzamide,
N-[1-[4-cyano-1-(5-cyano-2-pyridinyl)-3-(methylamino)-1H-pyrazol-5-yl]ethyl]-N-methyl-3,5-bis(trifluoromethyl)benzamide,
N-methyl-N-(1-(4-nitro-1-(pyrimidin-2-yl)-1H-pyrazol-5-yl)ethyl)-3,5-bis(trifluoromethyl)benzamide,
N-(1-(4-cyano-1-(5-cyanopyridin-2-yl)-1H-pyrazol-5-yl)ethyl)-N-methyl-3,5-bis(trifluoromethyl)benzamide,
3-chloro-N-(1-(4-cyano-1-(5-cyanopyridin-2-yl)-1H-pyrazol-5-yl)ethyl)-N-methyl-5-(trifluoromethyl)benzamide,
N-(1-(4-cyano-1-(pyrimidin-2-yl)-1H-pyrazol-5-yl)ethyl)-3,5-bis(trifluoromethyl)benzamide,
N-[1-[3-amino-4-cyano-1-[5-(4-morpholinylcarbonyl)-2-pyridinyl]-1H-pyrazol-5-yl]ethyl]-N-methyl-3,5-bis(trifluoromethyl)benzamide,
N,N-dimethyl-6-(5-(1-(N-methyl-3,5-bis(trifluoromethyl)benzamido)ethyl)-4-(methylsulfonyl)-1H-pyrazol-1-yl)pyrimidine-4-carboxamide,
N-(1-(4-cyano-3-methoxy-1-(pyrimidin-2-yl)-1H-pyrazol-5-yl)ethyl)-3,5-bis(trifluoromethyl)benzamide,
N-methyl-N-(1-(4-(methylsulfonyl)-1-(pyrazin-2-yl)-1H-pyrazol-5-yl)ethyl)-3,5-bis(trifluoromethyl)benzamide,
N-(1-(4-nitro-1-(pyrimidin-2-yl)-1H-pyrazol-5-yl)ethyl)-3,5-bis(trifluoromethyl)benzamide, and
N-methyl-N-(1-(4-(methylsulfonyl)-1-(6-(trifluoromethyl)pyrimidin-4-yl)-1H-pyrazol-5-yl)ethyl)-3,5-bis(trifluoromethyl)benzamide.

17. A composition comprising a compound anyone of the foregoing claims and at least one additional component selected from the group consisting of surfactants, solid diluents and liquid diluents, said composition optionally further comprising at least one additional biologically active compound or agent.

18. The composition of claim 15 wherein the at least one additional biologically active compound or agent is selected from the group consisting of of abamectin, acephate, acequinocyl, acetamiprid, acrinathrin, afidopyropen, amidoflumet, amitraz, avermectin, azadirachtin, azinphos-methyl, bendiocarb, benfuracarb, bensultap, bifenthrin, bifenazate, bistrifluron, borate, bromantraniliprole, buprofezin, carbaryl, carbofuran, cartap, chlorantraniliprole, chlorfenapyr, chlorfluazuron, chloroprallethrin, chlorpyrifos, chlorpyrifos-methyl, chromafenozide, clofentezine, clothianidin, cyantraniliprole, cyclaniliprole, cyclobutrifluram, cycloprothrin, cycloxaprid, cyetpyrafen, cyflumetofen, cyfluthrin, beta-cyfluthrin, cyhalodiamide, cyhalothrin, gamma-cyhalothrin, lambda-cyhalothrin, cypermethrin, alpha-cypermethrin, zeta-cypermethrin, cyproflanilide, cyromazine, deltamethrin, diafenthiuron, diazinon, dichlorantraniliprole, dieldrin, diflovidazin, diflubenzuron, dimefluthrin, dimehypo, dimethoate, dinotefuran, diofenolan, emamectin, endosulfan, esfenvalerate, ethiprole, etofenprox, etoxazole, fenazaquin, fenbutatin oxide, fenitrothion, fenmezoditiaz, fenothiocarb, fenoxycarb, fenpyroximate, fenpropathrin, fenvalerate, fipronil, flometoquin, flonicamid, fluacrypyrim, fluazaindolizine, flubendiamide, fluchlordiniliprole, flucythrinate, flufenerim, flufenoxuron, flufenoxystrobin, fluensulfone, fluhexafon, flupentiofenox, fluopyram, flupyrimin, flupyradifurone, fluvalinate, tau-fluvalinate, fluxametamide, fonofos, formetanate, fosthiazate, halofenozide, heptafluthrin, hexaflumuron, hexythiazox, hydramethylnon, hydroprene, imidacloprid, indazapyroxamet (N-(1-methylcyclopropyl)-2-(3-pyridinyl)-2H-indazole-4-carboxamide), indoxacarb, insecticidal soaps, isofenphos, isocycloseram, kappa-tefluthrin, kinoprene, lufenuron, malathion, meperfluthrin, metaflumizone, metaldehyde, methamidophos, methidathion, methiocarb, methomyl, methoprene, methoxychlor, methoxyfenozide, metofluthrin, monocrotophos, monofluorothrin, nicofluprole, nicotine, N-[1,1-dimethyl-2-(methylthio)ethyl]-7-fluoro-2-(3-pyridinyl)-2H-indazole-4-carboxamide, N-[1,1-dimethyl-2-(methylsulfinyl)ethyl]-7-fluoro-2-(3-pyridinyl)-2H-indazole-4-carboxamide, N-[1,1-dimethyl-2-(methylsulfonyl)ethyl]-7-fluoro-2-(3-pyridinyl)-2H-indazole-4-carboxamide, N-(1-methylcyclopropyl)-2-(3-pyridinyl)-2H-indazole-4-carboxamide, N-[1-(difluoromethyl)cyclopropyl]-2-(3-pyridinyl)-2H-indazole-4-carboxamide, nitenpyram, nithiazine, novaluron, noviflumuron, oxamyl, oxazosulfyl, parathion, parathion-methyl, permethrin, phorate, phosalone, phosmet, phosphamidon, pirimicarb, profenofos, profluthrin, propargite, protrifenbute, pyflubumide, pymetrozine, pyrafluprole, pyrethrins (pyrethrum), pyridaben, pyridalyl, pyrifluquinazon, pyrimidifen, pyriminostrobin, pyriprole, pyriproxyfen, rotenone, ryanodine, silafluofen, spidoxamat, spinetoram, spinosad, spirobudifen, spirodiclofen, spiromesifen, spirotetramat, sulprofos, sulfoxaflor, tebufenozide, tebufenpyrad, teflubenzuron, tefluthrin, kappa-tefluthrin, terbufos, tetrachlorantraniliprole, tetrachlorvinphos, tetramethrin, tetramethylfluthrin, tetraniliprole, thiacloprid, thiamethoxam, thiodicarb, thiosultap, thiosultap-sodium, tiorantraniliprole, tioxazafen, tolfenpyrad, tralomethrin, triazamate, trichlorfon, trifluenfuronate, triflumezopyrim, triflumuron, tyclopyrazoflor, Bacillus sphaericus, Bacillus thuringiensis delta-endotoxins, entomopathogenic bacteria, entomopathogenic viruses and entomopathogenic fungi, botanical essence including synthetic extracts and unrefined oils, RNA interference mediated target suppressors.

19. A method for controlling and combating an invertebrate pest comprising contacting the invertebrate pest or its environment with a biologically effective amount of a compound of anyone of claims 1-16.

20. A treated seed comprising a compound of of any one of claims 1-16 in an amount of from about 0.0001 to 1% by weight of the seed before treatment.
